(19) United States
(12) Patent Application Publication
(10) Pub. No.: US 2021/0038166 A1
(43) Pub. Date: Feb. 11, 2021

(54) METHOD AND SYSTEM FOR PREDICTING CHILDHOOD OBESITY

(71) Applicant: Yeda Research and Development Co. Ltd., Rehovot (IL)

(72) Inventors: Eran SEGAL, Ramat-HaSharon (IL); Smadar SHILO, Rehovot (IL); Hagai ROSSMAN, Rehovot (IL)

(73) Assignee: Yeda Research and Development Co. Ltd., Rehovot (IL)

(21) Appl. No.: 16/985,375

(22) Filed: Aug. 5, 2020

Related U.S. Application Data
(60) Provisional application No. 62/882,623, filed on Aug. 5, 2019.

(51) Int. Cl. A61B 5/00; A61B 5/145; G06N 20/00; G16H 10/60; G16H 10/20

(52) U.S. Cl. CPC A61B 5/7275; A61B 5/48; A61B 5/14507; A61B 2503/045; G16H 10/60; G16H 10/20; A61B 2503/06; G06N 20/00

(57) ABSTRACT

A method of predicting likelihood for childhood obesity, comprises: obtaining a plurality of parameters, wherein at least a few of the parameters characterize an infant or toddler subject. A machine learning procedure trained for predicting likelihoods for childhood obesity is feed with the plurality of parameters. An output indicative of a likelihood that the infant or toddler subject is expected to develop childhood obesity is received from the procedure. The output is related non-linearly to the parameters.

### RELATED APPLICATIONS

This application claims the benefit of priority under 35 USC 119(e) of U.S. Provisional Patent Application No. 62/882,623 filed on Aug. 5, 2019, the contents of which are all incorporated by reference as if fully set forth herein in their entirety.

### FIELD AND BACKGROUND OF THE INVENTION

The present invention, in some embodiments thereof, relates to medicine and, more particularly, but not exclusively, to a method and system for predicting childhood obesity.

Over the past decades, the prevalence of childhood obesity has rapidly increased worldwide. A global analysis demonstrated that in 2016, 50 million girls and 74 million boys worldwide were obese, making it a global public health crisis. Obese children are very likely to have obesity persist into adulthood. Childhood obesity is associated with elevated blood pressure and lipids, and increased risk of diseases, such as asthma, type 2 diabetes, arthritis, and cardiovascular diseases at a later stage of life. Furthermore, childhood obesity can have a negative psycho-social effect.

Preventing excess weight gain in children is important for numerous reasons. Pediatric obesity is a multisystem disease that can greatly impact a child's physical and mental health. It is associated with a greater risk for premature mortality and earlier onset of chronic disorders such as hypertension, dyslipidemia, ischemic heart disease and type 2 diabetes, with insulin resistance identified in obese children as young as 5 years of age. Furthermore, there is currently an underestimation of obesity by parents and physicians and there is currently little guidance for health care professionals to identify infants at risk. Additionally, young age is a suitable time period for intervention, as it is associated with more beneficial long-term outcomes after lifestyle modifications.

### SUMMARY OF THE INVENTION

According to an aspect of some embodiments of the present invention there is provided a method of predicting likelihood for childhood obesity. The method comprises: obtaining a plurality of parameters, wherein at least a few of the parameters characterize an infant or toddler subject; accessing a computer readable medium storing a machine learning procedure trained for predicting likelihoods for childhood obesity; feeding the procedure with the plurality of parameters; and receiving from the procedure an output indicative of a likelihood that the infant or toddler subject is expected to develop childhood obesity, wherein the output is related non-linearly to the parameters.

According to some embodiments of the invention the plurality of parameters comprises at least one parameter extracted from an electronic health record associated with the infant or toddler subject.

According to some embodiments of the invention the method comprises presenting to a user, by a user interface, a questionnaire and a set of questionnaire controls, receiving a set of response parameters entered by the user using the questionnaire controls, wherein the plurality of parameters comprises the response parameters.

According to some embodiments of the invention the plurality of parameters comprises at least one parameter extracted from a body liquid test applied to the infant or toddler subject.

According to some embodiments of the invention the plurality of parameters comprises at least one parameter characterizing a parent or a sibling of the infant or toddler subject.

According to some embodiments of the invention the at least one parameter characterizing the parent comprises a parameter extracted from a body liquid test applied to the parent or sibling.

According to some embodiments of the invention the plurality of parameters comprises at least one parameter extracted from a diagnosis previously recorded for the subject.

According to some embodiments of the invention the plurality of parameters comprises at least one parameter indicative of a pharmaceutical prescribed for the infant or toddler subject.

According to some embodiments of the invention the infant or toddler subject is less than two years of age.

According to some embodiments of the invention the infant or toddler subject is not obese. According to some embodiments of the invention the method wherein the infant or toddler subject has a normal weight. According to some embodiments of the invention the plurality of parameters comprises a weight-for-length score of the infant or toddler subject.

According to some embodiments of the invention the plurality of parameters comprise a weight of the infant or toddler subject at age of from about 4 to about 6 months, a weight of the infant or toddler subject at age of from about 12 to about 16 months, and a weight of the infant or toddler subject at age of from about 18 to about 22 months.

According to some embodiments of the invention the plurality of parameters comprises a parameter pertaining to a body-mass-index of a sibling of the infant or toddler subject.

According to some embodiments of the invention the plurality of parameters comprises a parameter pertaining to a body-mass-index of a father of the infant or toddler subject.

According to some embodiments of the invention the plurality of parameters comprises a result of a hemoglobin concentration test applied to the infant or toddler subject.

According to some embodiments of the invention the wherein the plurality of parameters comprises a result of a mean platelet volume test applied to the infant or toddler subject.

According to some embodiments of the invention the plurality of parameters comprises at least 10 or at least 20 or at least 30 or at least 40 or at least 50 or at least 100 or at least 200 or at least 300 or at least 400 or at least 500 or more of the parameters listed in Table 1.1.

According to some embodiments of the invention the plurality of parameters comprises at least 10 or at least 12 or at least 14 or at least 16 of the parameters that are listed at lines 1-40 more preferably lines 1-30 more preferably lines 1-20 of Table 1.1.

According to some embodiments of the invention the plurality of parameters comprises at least 20 or at least 22 or at least 24 or at least 26 or at least 28 or at least 30 or at least 32 or at least 34 or at least 36 of the parameters that are listed at lines 1-50 more preferably lines 1-45 more preferably lines 1-40 of Table 1.1.

According to some embodiments of the invention the plurality of parameters comprises least 50 or at least 60 or at least 70 or at least 80 or at least 90 of the parameters that are listed at lines 1-300 more preferably lines 1-200 more preferably lines 1-100 of

Table 1.1.

According to an aspect of some embodiments of the present invention there is provided a method of predicting likelihood for childhood obesity. The method comprises: obtaining a plurality of parameters characterizing at least one of a parent and a sibling of an unborn subject; accessing a computer readable medium storing a machine learning procedure trained for predicting likelihoods for childhood obesity; feeding the procedure with the plurality of parameters; and receiving from the procedure an output indicative of a likelihood that the unborn subject is expected to develop childhood obesity after birth, wherein the output is related non-linearly to the parameters.

According to some embodiments of the invention the plurality of parameters comprises at least one parameter extracted from an electronic health record associated with the at least one of the parent and the sibling.

According to some embodiments of the invention the method comprises presenting to a user, by a user interface, a questionnaire and a set of questionnaire controls, receiving a set of response parameters entered by the user using the questionnaire controls, wherein the plurality of parameters comprises the response parameters.

According to some embodiments of the invention the plurality of parameters comprises at least one parameter extracted from a body liquid test applied to the at least one of the parent and the sibling.

According to some embodiments of the invention the plurality of parameters comprises a parameter pertaining to a body-mass-index of the sibling.

According to some embodiments of the invention the plurality of parameters comprises a parameter pertaining to a body-mass-index of a father of the unborn subject.

According to some embodiments of the invention the plurality of parameters comprises at least 10 or at least 20 or at least 30 or at least 40 or at least 50 or at least 100 or at least 200 or at least 300 or at least 400 or at least 500 or at least 1,000 or at least 1,500 or more of the parameters listed in Table 1.2.

According to some embodiments of the invention the plurality of parameters comprises at least 10 or at least 12 or at least 14 or at least 16 of the parameters that are listed at lines 1-40 more preferably lines 1-30 more preferably lines 1-20 of Table 1.2.

According to some embodiments of the invention the plurality of parameters comprises at least 20 or at least 22 or at least 24 or at least 26 or at least 28 or at least 30 or at least 32 or at least 34 or at least 36 of the parameters that are listed at lines 1-50 more preferably lines 1-45 more preferably lines 1-40 of Table 1.2.

According to some embodiments of the invention the plurality of parameters comprises least 50 or at least 60 or at least 70 or at least 80 or at least 90 of the parameters that are listed at lines 1-300 more preferably lines 1-200 more preferably lines 1-100 of Table 1.2.

According to an aspect of some embodiments of the present invention there is provided a method of predicting likelihood for childhood obesity. The method comprises: presenting on a user interface a questionnaire and a set of questionnaire controls, and receiving from the user interface a set of response parameters entered using the questionnaire controls, wherein the set of response parameters characterizes an infant or toddler subject; accessing a computer readable medium storing a machine learning procedure trained for predicting likelihoods for childhood obesity; feeding the procedure with the set of parameters; and receiving from the procedure an output indicative of a likelihood that the infant or toddler subject is expected to develop childhood obesity, wherein the output is related non-linearly to the parameters.

According to some embodiments of the invention the plurality of parameters comprises at least 10 or at least 20 or at least 30 or at least 40 or at least 50 or more of the parameters listed in Table 1.3.

According to some embodiments of the invention the plurality of parameters comprises at least 10 or at least 12 or at least 14 or at least 16 of the parameters that are listed at lines 1-40 more preferably lines 1-30 more preferably lines 1-20 of Table 1.3.

According to some embodiments of the invention the plurality of parameters comprises at least 20 or at least 22 or at least 24 or at least 26 of the parameters that are listed at lines 1-50 more preferably lines 1-40 more preferably lines 1-30 of Table 1.3.

According to an aspect of some embodiments of the present invention there is provided a method of predicting likelihood for childhood obesity. The method comprises: presenting on a user interface a questionnaire and a set of questionnaire controls, and receiving from the user interface a set of response parameters entered using the questionnaire controls, wherein the set of response parameters characterizes at least one of a parent and a sibling of an unborn subject; accessing a computer readable medium storing a machine learning procedure trained for predicting likelihoods for childhood obesity; feeding the procedure with the set of parameters; and receiving from the procedure an output indicative of a likelihood that the unborn subject is expected to develop childhood obesity after birth, wherein the output is related non-linearly to the parameters.

According to some embodiments of the invention the plurality of parameters comprises at least 5 or at least 10 or at least 15 or more of the parameters listed in Table 1.4.

According to some embodiments of the invention the plurality of parameters comprises at least 5 or at least 10 of the parameters that are listed at lines 1-15 of Table 1.4.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart diagram of a method suitable for predicting likelihood for childhood obesity, according to various exemplary embodiments of the present invention.

FIG. 2 is a schematic illustration of a client-server configuration which can be used according to some embodiments of the present invention for predicting likelihood for childhood obesity, according to some embodiments of the present invention.

FIG. 3 is a diagram illustrating a dataset of nationwide health records used in a study directed to a prediction of childhood obesity and an analysis of risk, according to some embodiments of the present invention.

FIGS. 4A-D show BMI dynamics in early childhood, as obtained in experiments performed according to some embodiments of the present invention. FIG. 4A shows mean BMI z-score for children who were obese (upper curve) versus non obese (lower curve) at 13 years of age. FIG. 4B shows mean change in annual BMI-scores for the same groups of children. Shaded areas are 95% bootstrapped confidence intervals. FIG. 4C shows obesity status transition of the study cohort. Left side: distribution of obesity status at the last available routine checkup before 2 years of age. Right side: distribution of obesity status at 5-6 years of age. Transitions from different obesity states between these two time points are presented. FIG. 4D shows distribution of obesity status at infancy for obese 5-6 years old children.

FIGS. 5A-D show evaluation of obesity prediction model constructed in accordance with some embodiments of the present invention. FIG. 5A shows ROC curve of the model of the present embodiments (solid line) and a baseline model based on the last available routine checkup measurement (dashed). The dots and percentages represent different decision probability thresholds. FIG. 5B is calibration curve. The dots represents deciles of predicted probabilities. The dotted diagonal line represents an ideal calibration. The histogram at the bottom represents predicted probabilities of normal-weight children and obese children. FIG. 5C shows a Precision-Recall curve. The Baseline model is marked with an X. Threshold percentiles are marked on the curves. FIG. 5D shows decision curve analysis containing different treatment strategies of the model according to some embodiments of the present invention (solid curve) and the baseline model (dashed curve). Strategies of treating all (dashed line), treating none (dotted line) and the perfect hypothetical predictor (dot-dash line) are also presented. Abbreviations: auPR/auROC—Area under the PR/ROC curve, PPV—positive predictive value, PR—Precision-Recall, ROC—Receiver-Operator-Characteristic

FIGS. 6A-C show discrimination performances of the obesity prediction model in accordance with some embodiments of the present invention. The discrimination performances are represented by Precision-Recall (auPR) according to last measured WFL percentile (FIG. 6A), different subpopulations of children (FIG. 6B), and the child's age (0-24 months) (FIG. 6C). Abbreviations: auPR—Area under the PR curve, PR—Precision-Recall, WFL—weight for length.

FIGS. 7A-H show interpretation of the model of the present embodiments. FIG. 7A shows Shapley values of different groups of features. FIGS. 7B-H are plots showing in the lower part a histogram of the distribution of a feature in the data and in the upper part a dependence plot of the predicted relative risk for obesity at 5-6 years of age versus the value of the feature for child last WFL z-score (FIG. 7B), child birth weight (FIG. 7C), siblings mean BMI z-score (FIG. 7D), maternal and paternal mean BMI (FIG. 7E); maternal 50 g GCT results during pregnancy (FIG. 7F), duration of antibiotic therapy calculated by the summation of the days in which the child was issued an antibiotics treatment (FIG. 7G), and Child North African Ethnicity index (FIG. 7H). Abbreviations: GCT—glucose challenge test, WFL—Weight-for-Length, y/o—years old.

FIGS. 8A and 8B show results of applying the childhood obesity prediction model of the present embodiments prior to 2 years of age. FIG. 8A shows auPR curve for prediction models of obesity at 5-6 years of age based on features that were extracted up to a predefined endpoint age, ranging from pre-birth to 2 years of age of note, auPR of the prediction model pre-birth and at birth overlap. The baseline model was defined as last routine checkup WFL z-score. FIG. 8B shows relative importance of groups of features for the prediction models, calculated by normalizing to the sum of mean absolute SHAP values for each model. “Others” sums up non-anthropometric or demographic features such as laboratory tests and drug features. Abbreviations: auPR—Area under the PR curve, PR—Precision-Recall, WFL—weight for length

### DESCRIPTION OF SPECIFIC EMBODIMENTS OF THE INVENTION

The present invention, in some embodiments thereof, relates to medicine and, more particularly, but not exclusively, to a method and system for predicting childhood obesity.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

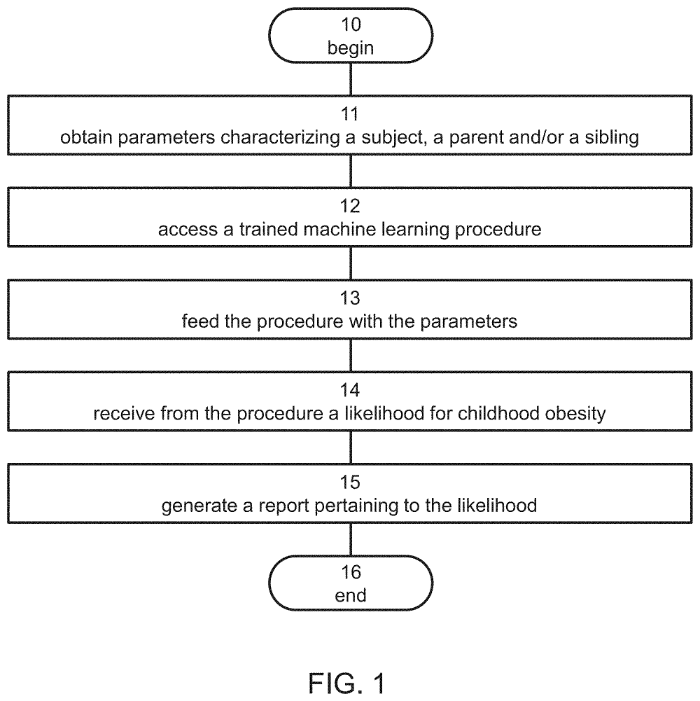

FIG. 1 is a flowchart diagram of a method suitable for predicting likelihood for childhood obesity, according to various exemplary embodiments of the present invention. It is to be understood that, unless otherwise defined, the operations described hereinbelow can be executed either contemporaneously or sequentially in many combinations or orders of execution. Specifically, the ordering of the flowchart diagrams is not to be considered as limiting. For example, two or more operations, appearing in the following description or in the flowchart diagrams in a particular order, can be executed in a different order (e.g., a reverse order) or substantially contemporaneously. Additionally, several operations described below are optional and may not be executed.

The processing operations of the present embodiments can be embodied in many forms. For example, they can be embodied in on a tangible medium such as a computer for performing the operations. They can be embodied on a computer readable medium, comprising computer readable instructions for carrying out the method operations. They can also be embodied in electronic device having digital computer capabilities arranged to run the computer program on the tangible medium or execute the instruction on a computer readable medium.

Computer programs implementing the method according to some embodiments of this invention can commonly be distributed to users on a distribution medium such as, but not limited to, CD-ROM, flash memory devices, flash drives, or, in some embodiments, drives accessible by means of network communication, over the internet (e.g., within a cloud environment), or over a cellular network. From the distribution medium, the computer programs can be copied to a hard disk or a similar intermediate storage medium. The computer programs can be run by loading the computer instructions either from their distribution medium or their intermediate storage medium into the execution memory of the computer, configuring the computer to act in accordance with the method of this invention. Computer programs implementing the method according to some embodiments of this invention can also be executed by one or more data processors that belong to a cloud computing environment. All these operations are well-known to those skilled in the art of computer systems. Data used and/or provided by the method of the present embodiments can be transmitted by means of network communication, over the internet, over a cellular network or over any type of network, suitable for data transmission.

The method according to preferred embodiments of the present invention can be embedded into healthcare systems and may allow identification and implementation of prevention strategies for children at high risk for obesity.

The method begins at 10 and continues to 11 at which a plurality of parameters characterizing is obtained. The inventors discovered that the likelihood for childhood obesity can be predicted both for infant or toddler subjects and for unborn subjects, e.g., during the pregnancy of a female carrying the unborn subject.

As used herein “infant” refers to an individual not more that 1 year of age, and “toddler” refers to an individual above 1 year of age and not more than 3 years of age”

Thus, in some embodiments of the present invention the method predicts likelihood that an infant or toddler subject is expected to develop childhood obesity, and in some embodiments of the present invention the method predicts unborn subject is expected to develop childhood obesity after birth. When the subject is an infant or toddler subject he or she is preferably of less than two years of age. The method of the present embodiments is typically used for estimating the likelihood that the subject is expected to develop childhood obesity at age greater than the toddler age, e.g., more than 4 years of age, for example, from about 5 to about 6 years of age.

When the subject is an infant or toddler subject, at least one of the parameters that are obtained at 11, more preferably more than one of these parameters, more preferably at least 10 or at least 20 or at least 30 or at least 40 or at least 50 or at least 100 or at least 200 or at least 300 or at least 400 or at least 500 or more of the parameters are extracted from an electronic health record associated with the subject. Parameters extracted from an electronic health record can include, but are not limited to, anthropometric parameters (e.g., height, weight, body mass index, weight-for-length score), blood pressure measurements, blood and urine laboratory tests, diagnoses recorded by physicians, and/or pharmaceuticals prescribed to the subject.

In some embodiments of the present invention at least one of the parameters that are obtained at 11, more preferably more than one of these parameters, more preferably at least 10 or at least 20 or at least 30 or at least 40 or at least 50 or at least 100 or at least 200 or at least 300 or at least 400 or at least 500 or more of the parameters are extracted from an electronic health record associated with a parent (mother and/or father) and/or a sibling (brother and or sister) of the subject. These parameters can include any of the aforementioned parameters associated with the subject, except that they describe the respective parent or sibling (e.g., anthropometric parameters, blood pressure measurements, blood and urine laboratory tests, diagnoses, pharmaceuticals).

When the subject is an unborn subject, there are typically no parameters that describe the subject itself, and so the parameters that are obtained at 11 are typically associated with a parent (mother and/or father) and/or a sibling (brother and or sister) of the subject, as further detailed hereinabove.

A list of parameters from which the parameters can be selected when the subject is an infant or toddler subject is provided in Table 1.1 of the Examples section that follows, and list of parameters from which the parameters can be selected when the subject is an unborn subject is provided in Table 1.2 of the Examples section that follows. In some embodiments of the present invention at least 10 or at least 20 or at least 30 or at least 40 or at least 50 or at least 100 or at least 200 or at least 300 or at least 400 or at least 500 are selected from the parameters listed in Table 1.1 (for an infant or toddler subject) or Table 1.2 (for an unborn subject). Preferably, but not necessarily, at least 10 or at least 12 or at least 14 or at least 16 of the parameters are selected from the parameters that are listed at lines 1-40 more preferably lines 1-30 more preferably lines 1-20 of Table 1.1 (for an infant or toddler subject) or Table 1.2 (for an unborn subject). In some embodiments, at least 20 or at least 22 or at least 24 or at least 26 or at least 28 or at least 30 or at least 32 or at least 34 or at least 36 of the parameters are selected from the parameters that are listed at lines 1-50 more preferably lines 1-45 more preferably lines 1-40 of Table 1.1 (for an infant or toddler subject) or Table 1.2 (for an unborn subject). In some embodiments, at least 50 or at least 60 or at least 70 or at least 80 or at least 90 of the parameters are selected from the parameters that are listed at lines 1-300 more preferably lines 1-200 more preferably lines 1-100 of Table 1.1 (for an infant or toddler subject) or Table 1.2 (for an unborn subject).

Also contemplated are embodiments in which the parameters are selected from a set of response parameters that are provided by a person on behalf of the subject (e.g., a parent, a sibling, etc.), by responding to a questionnaire presented to the person. These parameters can include anthropometric parameters (e.g., height, weight, body mass index, weight-for-length score), one or more parameters indicative of the age of the subject (if born), and one or more parameters indicative of the ethnicity of the subject. A list of parameters which can be provided by responding to the questionnaire is provided in Table 1.3 for the case in which the subject is an infant or toddler subject, and in Table 1.4 for the case in which the subject is an unborn subject.

In some embodiments of the present invention the parameters include only parameters extracted from one or more electronic health records, in some embodiments of the present invention the parameters include only response parameters that are provided on behalf of the subject, and in some embodiments of the present invention the parameters include both parameters extracted from electronic health record(s) and response parameters that are provided by the subject or on her behalf.

In some embodiments of the present invention the electronic health record(s) include a record that is associated with the subject, in some embodiments of the present invention parameters the electronic health record(s) include records that are associated with at least one of a parent and a sibling of the subject, and in some embodiments of the present invention the electronic health record(s) include at least one record that is associated with the subject, and at least one record that is associated with a parent and/or a sibling of the subject.

The number of parameters that are extracted from the electronic health record(s) associated is preferably at least 10 or at least 20 or at least 30 or at least 40 or at least 50 or at least 100 or at least 200 or at least 300 or at least 400 or at least 500 or more. The number of response parameters that are provided by the subject or on her behalf is preferably 100 or less, or 80 or less, or 70 or less. The advantage of this embodiment is that a relative small number of parameter allows the subject to manually respond to the questionnaire at a relatively short time.

When the parameters include both parameters extracted from electronic health record(s), and response parameters that are provided on behalf of the subject, the number of parameters that are extracted from the electronic health record(s) is optionally and preferably significantly larger (e.g., at least 2 or at least 3 or at least 4 or at least 5 or at least 6 or at least 7 or at least 8 or at least 9 or at least 10 times larger) than the number of response parameters that are provided on behalf of the subject.

In some embodiments of the present invention at least one of the parameters is extracted from a body liquid test applied to the infant or toddler subject. Representative examples of body liquid tests from which a parameter can extracted from a body liquid test applied to the infant or toddler subject according to some embodiments of the present invention include, without limitation, Albumin test, Alk. phosphatase test, Atypical lymph. %-dif test, Atypical lymph-dif test, Basophils percentage (Baso %) test, Basophils (Baso abs) test, Bilirubin total test, Bilirubin-direct test, Calcium test, Chloride test, Cholesterol test, C-reactive protein test, Creatinine test, Eos % test, Eos.abs test, Eosinophils abs-dif test, Eosinophils %-dif test, Ferritin test, Gamma glutamyl transferase (Ggt) test, Glucose test, Got (ast) test, Alanine aminotransferase (Gpt (alt)) test, hemoglobin concentration (Hb) test, Hematocrit (Hct) test, Hematocrit/hemoglobin (Hct/hgb) ratio test, Hyper % test, Hypochromic red cells (Hypo %) test, Iron test, Ldh test, Luc abs test, Luc % test, Lym % test, Lymp.abs test, Lymphocytes %-dif test, Lymphocytes abs-dif test, Macro % test, Mean cell hemoglobin (Mch) test, mean hemoglobin concentration (Mchc) test, mean corpuscular volume (Mcv) test, Micro % test, Micro %/hypo % test, Mono % test, Mono.abs test, Monocytes abs-dif test, Monocytes %-dif test, mean platelet volume (Mpv) test, Mpxi test, Neut % test, Neut.abs test, Neutrophils abs-dif test, Neutrophils %-dif test, Pct test, Pdw test, Phosphorus test, platelet count blood (Plt) test, Potassium test, Protein-total test, Rbc test, red cell distribution width (Rdw) test, Red blood cell distribution width presented as the coefficient of variation (Rdw-cv) test, Sodium test, Stabs %-dif test, Stabs abs-dif test, T4-free test, Transferrin test, Triglycerides test, Thyroid-stimulating hormone (Tsh) test, Urea test, Uric acid test, and white blood cells (Wbc) test.

In some embodiments of the present invention at least one of the parameters is extracted from a body liquid test applied to the mother of the infant or toddler subject during pregnancy of the mother with the infant or toddler subject. Representative examples of body liquid tests from which a parameter can extracted from a body liquid test applied to the mother according to some embodiments of the present invention include, without limitation, Albumin, Alk. phosphatase, Alpha fetoprotein tm, Amylase, Aptt-r, Aptt-sec, Baso %, Baso abs, Bilirubin indirect, Bilirubin total, Bilirubin-direct, Blood type, Calcium, Chloride, Cholesterol-hdl, Cholesterol, Cholesterol/hdl, Cholesterol-ldl calc, Ck-creat.kinase(cpk), Cmv igg, Control ptt, Creatinine, Dhea sulphate, Eos %, Eos.abs, Eosinophils abs-dif, Eosinophils %-dif, Esr, Estradiol (e-2), Ferritin, Fibrinogen calcu, Fibrinogen, Folic acid, Fsh, Ggt, Globulin, Glom.filtr.rate, Glucose (gtt) 0′, Glucose (gtt) 120′, Glucose (gtt) 180′, Glucose (gtt) 60′, Glucose 50 g, Glucose, Got (ast), Gpt (alt), Hb, Hba, Hba2, Hbf, Hct, Hct/hgb ratio, Hdw, Hemoglobin a, Hemoglobin alc %, Hepatitis bs ab, Hyper %, Hypo %, Iron, Ldh, Lh, Li, Luc abs, Luc %, Lym %, Lymp.abs, Lymphocytes %-dif, Lymphocytes abs-dif, Macro %, Magnesium, Mch, Mchc, Mcv, Micro %, Micro %/hypo %, Mono %, Mono.abs, Monocytes abs-dif, Monocytes %-dif, Mpv, Mpxi, Neut %, Neut.abs, Neutrophils abs-dif, Neutrophils %-dif, Non-hdl_cholesterol, Normoblast. %, Normoblast.abs, Pct, Pdw, Phosphorus, Plt, Potassium, Progesterone, Prolactin, Protein-total, Pt %, Pt-inr, Pt-sec, Rbc, Rdw, Rdw-cv, Rubella ab igg, Sodium, Stabs %-dif, Stabs abs-dif, T3-free, T4-free, Toxoplasma igg, Transferrin, Triglycerides, Tsh, Urea, Uric acid, Vitamin b12, Vitamin d (25-oh), and Wbc.

In some embodiments of the present invention at least one of the parameters is extracted from a test applied to the mother of the infant or toddler subject prior to the pregnancy of the mother with the infant or toddler subject. Representative examples such tests include, without limitation, 17-oh-progesterone, Albumin, Alk. phosphatase, Aly, Aly %, Amylase, Androstenedione, Anti cardiolipin igg, Anti cardiolipin igm, Antithrombin-iii, Aptt-r, Aptt-sec, Baso %, Baso abs, Bilirubin indirect, Bilirubin total, Bilirubin-direct, Blood type, BMI, Ca-125, Calcium, Chloride, Cholesterol-hdl, Cholesterol, Cholesterol/hdl, Cholesterol-ldl calc, Ck-creat.kinase(cpk), Cmv igg, Complement c3, Complement c4, Control ptt, Cortisol-blood, C-reactive protein, Creatinine, Dhea sulphate, Eos %, Eos.abs, Eosinophils %-dif, Esr, Estradiol (e-2), Ferritin, Fibrinogen calcu, Fibrinogen, Folic acid, Free androgen index, Fsh, Ggt, Globulin, Glom.filtr.rate, Glucose 50 g, Glucose, Got (ast), Gpt (alt), Hb, Hba2, Hbf, Hct, Hct/hgb ratio, Hdw, Hemoglobin a, Hemoglobin alc %, Hepatitis bs ab, Hyper %, Hypo %, Iga, Iron, Ldh, Lh, Lic, Lic %, Luc abs, Luc %, Lym %, Lymp.abs, Lymphocytes %-dif, Lymphocytes abs-dif, Macro %, Magnesium, Mch, Mchc, Mcv, Micro %, Micro %/hypo %, Mono %, Mono.abs, Monocytes abs-dif, Monocytes %-dif, Mpv, Mpxi, Neut %, Neut.abs, Neutrophils abs-dif, Neutrophils %-dif, Non-hdl_cholesterol, Normoblast. %, Normoblast.abs, Pct, Pdw, Phosphorus, Plt, Potassium, Progesterone, Prolactin, Protein c activity, Protein-total, Prot-s antigen (free, Pt %, Pt-inr, Pt-sec, Rbc, Rdw, Rdw-cv, Rubella ab igg, Shbg, Sodium, T3-free, T3-total, T4-free, Testosterone-total, Toxoplasma igg, Transferrin, Triglycerides, Tsh, Urea, Uric acid, Vitamin b12, Vitamin d (25-oh), Vldl, Wbc, and Weight.

In some embodiments of the present invention the plurality of parameters comprises a result of a blood glucose test applied to the mother of the subject.

In some embodiments of the present invention at least one of the parameters is extracted from a test applied to the father of the infant or toddler subject. Representative examples of such tests include, without limitation, Age at the birth of the subject, BMI count, BMI max, BMI mean, BMI median, BMI min, BMI standard deviation (std), Height count, Height max, Height mean, Height median, Height min, Height std, max Cholesterol-hdl, max Cholesterol, max Cholesterol/hdl, max Cholesterol-ldl calc, max Glucose, max Non-hdl_cholesterol, max Triglycerides, mean Cholesterol-hdl, mean Cholesterol, mean Cholesterol/hdl, mean Cholesterol-ldl calc, mean Glucose, mean Non-hdl_cholesterol, mean Triglycerides, median Cholesterol-hdl, median Cholesterol, median Cholesterol/hdl, median Cholesterol-ldl calc, median Glucose, median Non-hdl_cholesterol, median Triglycerides, min Cholesterol-hdl, min Cholesterol, min Cholesterol/hdl, min Cholesterol-ldl calc, min Glucose, min Non-hdl_cholesterol, min Triglycerides, std Cholesterol-hdl, std Cholesterol, std Cholesterol/hdl, std Cholesterol-ldl calc, std Glucose, std Non-hdl_cholesterol, std Triglycerides, Weight count, Weight max, Weight mean, Weight median, Weight min, and Weight std.

In some embodiments of the present invention one or more of the parameters is a result of a hemoglobin concentration test (Hb) applied to the subject.

In some embodiments of the present invention one or more of the parameters is a result of a mean platelet volume test (Mpv) applied to the subject.

In some embodiments of the present invention one or more of the parameters is a result of a Basophils percentage test (Baso %) applied to the subject.

In some embodiments of the present invention one or more of the parameters is a result of a red cell distribution width test (Rdw) applied to the subject.

In some embodiments of the present invention one or more of the parameters is a result of a platelet count blood test (plt) applied to the subject.

In some embodiments of the present invention the parameters comprise at least one parameter extracted from a clinical or hospital diagnosis previously recorded for the subject. Representative examples of clinical and hospital diagnoses which can be used as parameters according to some embodiments of the present invention include, without limitation, Abdominal pain, Abnormal loss of weight, Abnormal weight gain, Accident/injury; nos, Acquired deformities of other parts of limbs, Acute and unspecified inflammation of lacrimal passages, Acute bronchiolitis, Acute bronchitis, Acute conjunctivitis, Acute laryngitis, Acute laryngotracheitis, Acute lymphadenitis, Acute myringitis without mention of otitis media, Acute nasopharyngitis (common cold), Acute nonsuppurative otitis media, Acute pharyngitis, Acute suppurative otitis media, Acute tonsillitis, Acute upper respiratory infections of multiple or unsp.sites, Acute upper respiratory infections of unspecified site, Agranulocytosis, Allergic rhinitis, Allergy, unspecified, not elsewhere classified, Allergy/allergic react nos, Anal fissure, Anemia other/unspecified, Anorexia, Asthma, Asthma, unspecified, Atopic dermatitis/eczema, Benign neoplasm of skin, site unspecified, Blepharitis, Blisters with epidermal loss,burn 2nd.deg.unspecified site, Bronchopneumonia, organism unspecified, Candidiasis of mouth, Candidiasis of skin and nails, Candidiasis of unspecified site, Cellulitis and abscess of finger, Cellulitis and abscess of unspecified sites, Chronic rhinitis, Chronic serous otitis media, Colitis, enteritis, gastroenteritis presumed infectious origin, Congenital anomalies of lower limb, including pelvic girdle, Congenital dislocation of hip, Congenital musculoskeletal deformities of sternocleidomastoid, Constipation, Contact dermatitis and other eczema, Contact dermatitis and other eczema, unspecified cause, Contusion of unspecified site, Convulsions, Cough, Croup, Delivery in a completely normal case, Dermatitis due to food taken internally, Dermatophytosis of the body, Diaper or napkin rash, Diarrhea, Diseases and other conditions of the tongue, Disorders relating to other preterm infants, Dyspnea and respiratory abnormalities, Enlargement of lymph nodes, Enteritis due to specified virus, Enterobiasis, Esophagitis, Feeding difficulties and mismanagement, Fever, Gastrointestinal hemorrhage, Hand, foot, and mouth disease, Hearing complaints, Hearing loss, Hemangioma of unspecified site, Herpangina, Hip symptoms/complaints, Hydrocele, Hydronephrosis, Hypermetropia, Hypertrophy of tonsils and adenoids, Impetigo, Infectious colitis, enteritis, and gastroenteritis, Infectious diarrhea, Infectious mononucleosis, Infective otitis externa, Influenza, Inguinal hernia, without mention of obstruction or gangrene, Injuries, Insect bite, Insect bite, nonvenomous face, neck, scalp without infection, Intestinal malabsorption, Iron deficiency anemia, unspecified, Irritable infant, Jaundice, unspecified, not of newborn, Laceration/cut, Lack of coordination, Lack of expected normal physiological development, Late effect of injury to cranial nerve, Laxity of ligament, Nausea and vomiting, Nervousness, Nonsuppurative otitis media, not specified as acute or chronic, Open wound of face, without mention of complication, Oral aphthae, Otalgia, Other and unspec.noninfectious gastroenteritis and colitis, Other and unspecified chronic nonsuppurative otitis media, Other and unspecified injury to unspecified site, Other atopic dermatitis and related conditions, Other diseases of conjunctiva due to viruses and chlamydiae, Other diseases of nasal cavity and sinuses, Other serum reaction, not elsewhere classified, Other specified disease of white blood cells, Other specified erythematous conditions, Other specified viral exanthemata, Other speech disturbance, Other symptoms involving digestive system, Other viral diseases; nos, Otorrhea, Pneumonia, Pneumonia, organism unspecified, Posttraumatic wound infection not elsewhere classified, Premat/immature liveborn infant, Rash and other nonspecific skin eruption, Seborrhea, Seborrheic dermatitis, unspecified, Serous otitis media;glue, Sleep disturbances, Sneezing/nasal congestion, Stenosis and insufficiency of lacrimal passages, Stomatitis, Strabismus and other disorders of binocular eye movements, Stridor, Teething syndrome, Tongue tie, Torticollis, unspecified, U.r.i. (head cold), Umbilical hernia without mention of obstruction or gangrene, Undescended testicle, Unsp.adv.effect of drug,medicinal/biological substance n.e.s., Unsp.viral infect.in conditions classif.elsewhere, unsp.site, Unspecified fetal and neonatal jaundice, Unspecified otitis media, Urinary tract infection, site not specified, Urticaria, Varicella without mention of complication, Viral exanthem, unspecified, Viral pneumonia, Volume depletion disorder, Vomiting (excl.preg. w06), and Wheezing baby syndrome.

In some embodiments of the present invention the parameters comprise at least one parameter indicative of a pharmaceutical prescribed for the subject. Representative examples of prescribed pharmaceuticals which can be used as parameters according to some embodiments of the present invention include, without limitation, Aciclovir, Ahiston drop cd, Amoxicillin, Azithromycin, Bethamethasone, Budesonide, Cefaclor, Cefalexin, Ceftriaxone, Cefuroxime, Co-amoxiclav cd, Co-trimoxazole cd, Desloratadine, Dimethindene, Erythromycin, Fluticasone, Ipratropium bromide, Ketotifen, Loratadine, Mebendazole, Metronidazole, Montelukast, Phenoxymethylpenicillin, Prednisolone, Prothiazine/promethazine expectorant cd, Ranitidine, Salbutamol, and Terbutaline.

In some embodiments of the present invention the parameters comprise at least one parameter indicative of a count of Salbutamol prescriptions provided for the infant or toddler subject.

In some embodiments of the present invention the parameters comprise at least one parameter indicative of a count of Bethamethasone prescriptions provided for the infant or toddler subject.

In some embodiments of the present invention the parameters comprise at least one parameter indicative of a count of Budesonide prescriptions provided for the infant or toddler subject.

In some embodiments of the present invention the parameters comprise at least one parameter indicative of a pharmaceutical prescribed for the mother of the subject. Representative examples of prescribed pharmaceuticals which can be used as parameters according to some embodiments of the present invention include, without limitation, Aciclovir, Amoxicillin, Anti-d (rh) immunoglobulin, Aspirin, Bethamethasone, Budesonide, Cabergoline, Carbamazepine, Cefalexin, Cefuroxime, Cetirizine, Choriogonadotropin alfa, Chorionic gonadotrophin, Ciprofloxacin, Citalopram, Clarithromycin, Clomifene, Clonazepam, Co-amoxiclav cd, Colchicine, Desloratadine, Desogestrel and ethinylestradiol, Desogestrel, Dexamethasone, Doxycycline, Drospirenone and ethinylestradiol, Dydrogesterone, Enoxaparin, Escitalopram, Estradiol, Famotidine, Fexofenadine, Fluconazole, Fluoxetine, Fluticasone, Follitropin alfa, Follitropin beta, Gestodene and ethinylestradiol, Human menopausal gonadotrophin, Ipratropium bromide, Lamotrigine, Lansoprazole, Levothyroxine sodium, Loratadine, Mebendazole, Medroxyprogesterone, Methylphenidate, Metronidazole, Nitrofurantoin, Norethisterone, Norgestimate and ethinylestradiol, Ofloxacin, Omeprazole, Paroxetine, Phenoxymethylpenicillin, Prednisone, Progesterone, Progyluton cd, Roxithromycin, Salbutamol, Seretide cd, Sertraline, Simvastatin, Symbicort/duoresp, and Triptorelin.

In some embodiments of the present invention the parameters comprise at least one parameter indicative of a pharmaceutical prescribed for the father of the subject. Representative examples of prescribed pharmaceuticals which can be used as parameters according to some embodiments of the present invention include, without limitation, Amlodipine, Atenolol, Atorvastatin, Bezafibrate, Bisoprolol, Cholesterol-hdl, Cholesterol, Cholesterol/hdl, Cholesterol-ldl calc, Enalapril, Glucose, Insulin glargine, Metformin and sitagliptin cd, Metformin, Nifedipine, Nifedipine-cd, Non-hdl_cholesterol, Pravastatin, Propranolol, Ramipril, Ramipril-hydrochlorothiazide cd, Rosuvastatin, Simvastatin, and Triglycerides.

In some embodiments of the present invention the parameters comprise at least one parameter extracted from a clinical or hospital diagnosis previously recorded for the father of subject. Representative examples of clinical and hospital diagnoses which can be used as parameters according to some embodiments of the present invention include, without limitation, Diabetes mellitus, unspecified Obesity, Obesity (BMI>30), other and unspecified hyperlipidemia, Essential hypertension, Morbid obesity, unspecified essential hypertension, Overweight (BMI<30), other abnormal glucose, Lipid metabolism disorder, Impaired fasting glucose, Disorders of lipoid metabolism, Diabetes mellitus without mention of complication, and Adult-onset type diabetes mellitus without complication.

Referring again to FIG. 1, the method proceeds to 12 at which a computer readable medium storing a machine learning procedure is accessed. The machine learning procedure is trained for predicting likelihoods for childhood obesity.

As used herein the term “machine learning” refers to a procedure embodied as a computer program configured to induce patterns, regularities, or rules from previously collected data to develop an appropriate response to future data, or describe the data in some meaningful way.

Representative examples of machine learning procedures suitable for the present embodiments, include, without limitation, clustering, association rule algorithms, feature evaluation algorithms, subset selection algorithms, support vector machines, classification rules, cost-sensitive classifiers, vote algorithms, stacking algorithms, Bayesian networks, decision trees, neural networks, instance-based algorithms, linear modeling algorithms, k-nearest neighbors (KNN) analysis, ensemble learning algorithms, probabilistic models, graphical models, logistic regression methods (including multinomial logistic regression methods), gradient ascent methods, singular value decomposition methods and principle component analysis.

Following is an overview of some machine learning procedures suitable for the present embodiments.

Support vector machines are algorithms that are based on statistical learning theory. A support vector machine (SVM) according to some embodiments of the present invention can be used for classification purposes and/or for numeric prediction. A support vector machine for classification is referred to herein as “support vector classifier,” support vector machine for numeric prediction is referred to herein as “support vector regression”.

An SVM is typically characterized by a kernel function, the selection of which determines whether the resulting SVM provides classification, regression or other functions. Through application of the kernel function, the SVM maps input vectors into high dimensional feature space, in which a decision hyper-surface (also known as a separator) can be constructed to provide classification, regression or other decision functions. In the simplest case, the surface is a hyper-plane (also known as linear separator), but more complex separators are also contemplated and can be applied using kernel functions. The data points that define the hyper-surface are referred to as support vectors.

The support vector classifier selects a separator where the distance of the separator from the closest data points is as large as possible, thereby separating feature vector points associated with objects in a given class from feature vector points associated with objects outside the class. For support vector regression, a high-dimensional tube with a radius of acceptable error is constructed which minimizes the error of the data set while also maximizing the flatness of the associated curve or function. In other words, the tube is an envelope around the fit curve, defined by a collection of data points nearest the curve or surface.

An advantage of a support vector machine is that once the support vectors have been identified, the remaining observations can be removed from the calculations, thus greatly reducing the computational complexity of the problem. An SVM typically operates in two phases: a training phase and a testing phase. During the training phase, a set of support vectors is generated for use in executing the decision rule. During the testing phase, decisions are made using the decision rule. A support vector algorithm is a method for training an SVM. By execution of the algorithm, a training set of parameters is generated, including the support vectors that characterize the SVM. A representative example of a support vector algorithm suitable for the present embodiments includes, without limitation, sequential minimal optimization.

In KNN analysis, the affinity or closeness of objects is determined. The affinity is also known as distance in a feature space between objects. Based on the determined distances, the objects are clustered and an outlier is detected. Thus, the KNN analysis is a technique to find distance-based outliers based on the distance of an object from its kth-nearest neighbors in the feature space. Specifically, each object is ranked on the basis of its distance to its kth-nearest neighbors. The farthest away object is declared the outlier. In some cases the farthest objects are declared outliers. That is, an object is an outlier with respect to parameters, such as, a k number of neighbors and a specified distance, if no more than k objects are at the specified distance or less from the object. The KNN analysis is a classification technique that uses supervised learning. An item is presented and compared to a training set with two or more classes. The item is assigned to the class that is most common amongst its k-nearest neighbors. That is, compute the distance to all the items in the training set to find the k nearest, and extract the majority class from the k and assign to item.

Association rule algorithm is a technique for extracting meaningful association patterns among features.

The term “association”, in the context of machine learning, refers to any interrelation among features, not just ones that predict a particular class or numeric value. Association includes, but it is not limited to, finding association rules, finding patterns, performing feature evaluation, performing feature subset selection, developing predictive models, and understanding interactions between features.

The term “association rules” refers to elements that co-occur frequently within the datasets. It includes, but is not limited to association patterns, discriminative patterns, frequent patterns, closed patterns, and colossal patterns.

A usual primary step of association rule algorithm is to find a set of items or features that are most frequent among all the observations. Once the list is obtained, rules can be extracted from them.

The aforementioned self-organizing map is an unsupervised learning technique often used for visualization and analysis of high-dimensional data. Typical applications are focused on the visualization of the central dependencies within the data on the map. The map generated by the algorithm can be used to speed up the identification of association rules by other algorithms. The algorithm typically includes a grid of processing units, referred to as “neurons”. Each neuron is associated with a feature vector referred to as observation. The map attempts to represent all the available observations with optimal accuracy using a restricted set of models. At the same time the models become ordered on the grid so that similar models are close to each other and dissimilar models far from each other. This procedure enables the identification as well as the visualization of dependencies or associations between the features in the data.

Feature evaluation algorithms are directed to the ranking of features or to the ranking followed by the selection of features based on their impact.

Information gain is one of the machine learning methods suitable for feature evaluation. The definition of information gain requires the definition of entropy, which is a measure of impurity in a collection of training instances. The reduction in entropy of the target feature that occurs by knowing the values of a certain feature is called information gain. Information gain may be used as a parameter to determine the effectiveness of a feature in explaining the response to the treatment. Symmetrical uncertainty is an algorithm that can be used by a feature selection algorithm, according to some embodiments of the present invention. Symmetrical uncertainty compensates for information gain's bias towards features with more values by normalizing features to a [0,1] range.

Subset selection algorithms rely on a combination of an evaluation algorithm and a search algorithm. Similarly to feature evaluation algorithms, subset selection algorithms rank subsets of features. Unlike feature evaluation algorithms, however, a subset selection algorithm suitable for the present embodiments aims at selecting the subset of features with the highest impact on predicting likelihood for childhood obesity, while accounting for the degree of redundancy between the features included in the subset. The benefits from feature subset selection include facilitating data visualization and understanding, reducing measurement and storage requirements, reducing training and utilization times, and eliminating distracting features to improve classification.

Two basic approaches to subset selection algorithms are the process of adding features to a working subset (forward selection) and deleting from the current subset of features (backward elimination). In machine learning, forward selection is done differently than the statistical procedure with the same name. The feature to be added to the current subset in machine learning is found by evaluating the performance of the current subset augmented by one new feature using cross-validation. In forward selection, subsets are built up by adding each remaining feature in turn to the current subset while evaluating the expected performance of each new subset using cross-validation. The feature that leads to the best performance when added to the current subset is retained and the process continues. The search ends when none of the remaining available features improves the predictive ability of the current subset. This process finds a local optimum set of features.

Backward elimination is implemented in a similar fashion. With backward elimination, the search ends when further reduction in the feature set does not improve the predictive ability of the subset. The present embodiments contemplate search algorithms that search forward, backward or in both directions. Representative examples of search algorithms suitable for the present embodiments include, without limitation, exhaustive search, greedy hill-climbing, random perturbations of subsets, wrapper algorithms, probabilistic race search, schemata search, rank race search, and Bayesian classifier.

A decision tree is a decision support algorithm that forms a logical pathway of steps involved in considering the input to make a decision.

The term “decision tree” refers to any type of tree-based learning algorithms, including, but not limited to, model trees, classification trees, and regression trees.

A decision tree can be used to classify the datasets or their relation hierarchically. The decision tree has tree structure that includes branch nodes and leaf nodes. Each branch node specifies an attribute (splitting attribute) and a test (splitting test) to be carried out on the value of the splitting attribute, and branches out to other nodes for all possible outcomes of the splitting test. The branch node that is the root of the decision tree is called the root node. Each leaf node can represent a classification (e.g., whether a particular parameter influences on the likelihood for childhood obesity) or a value (e.g., the predicted likelihood for childhood obesity). The leaf nodes can also contain additional information about the represented classification such as a confidence score that measures a confidence level in the represented classification (i.e., the accuracy of the prediction).

Regression techniques which may be used in accordance with some embodiments the present invention include, but are not limited to linear Regression, Multiple Regression, logistic regression, probit regression, ordinal logistic regression ordinal Probit-Regression, Poisson Regression, negative binomial Regression, multinomial logistic Regression (MLR) and truncated regression.

A logistic regression or logit regression is a type of regression analysis used for predicting the outcome of a categorical dependent variable (a dependent variable that can take on a limited number of values, whose magnitudes are not meaningful but whose ordering of magnitudes may or may not be meaningful) based on one or more predictor variables. Logistic regression may also predict the probability of occurrence for each data point. Logistic regressions also include a multinomial variant. The multinomial logistic regression model is a regression model which generalizes logistic regression by allowing more than two discrete outcomes. That is, it is a model that is used to predict the probabilities of the different possible outcomes of a categorically distributed dependent variable, given a set of independent variables (which may be real-valued, binary-valued, categorical-valued, etc.). For binary-valued variables, a cutoff between the 0 and 1 associations is typically determined using the Yuden Index.

A Bayesian network is a model that represents variables and conditional interdependencies between variables. In a Bayesian network variables are represented as nodes, and nodes may be connected to one another by one or more links. A link indicates a relationship between two nodes. Nodes typically have corresponding conditional probability tables that are used to determine the probability of a state of a node given the state of other nodes to which the node is connected. In some embodiments, a Bayes optimal classifier algorithm is employed to apply the maximum a posteriori hypothesis to a new record in order to predict the probability of its classification, as well as to calculate the probabilities from each of the other hypotheses obtained from a training set and to use these probabilities as weighting factors for future predictions of the likelihood for childhood obesity. An algorithm suitable for a search for the best Bayesian network, includes, without limitation, global score metric-based algorithm. In an alternative approach to building the network, Markov blanket can be employed. The Markov blanket isolates a node from being affected by any node outside its boundary, which is composed of the node's parents, its children, and the parents of its children.

Instance-based techniques generate a new model for each instance, instead of basing predictions on trees or networks generated (once) from a training set.

The term “instance”, in the context of machine learning, refers to an example from a dataset.

Instance-based techniques typically store the entire dataset in memory and build a model from a set of records similar to those being tested. This similarity can be evaluated, for example, through nearest-neighbor or locally weighted methods, e.g., using Euclidian distances. Once a set of records is selected, the final model may be built using several different techniques, such as the naive Bayes.

Neural networks are a class of algorithms based on a concept of inter-connected “neurons.” In a typical neural network, neurons contain data values, each of which affects the value of a connected neuron according to connections with predefined strengths, and whether the sum of connections to each particular neuron meets a predefined threshold. By determining proper connection strengths and threshold values (a process also referred to as training), a neural network can achieve efficient recognition of images and characters. Oftentimes, these neurons are grouped into layers in order to make connections between groups more obvious and to each computation of values. Each layer of the network may have differing numbers of neurons, and these may or may not be related to particular qualities of the input data.

In one implementation, called a fully-connected neural network, each of the neurons in a particular layer is connected to and provides input value to those in the next layer. These input values are then summed and this sum compared to a bias, or threshold. If the value exceeds the threshold for a particular neuron, that neuron then holds a positive value which can be used as input to neurons in the next layer of neurons. This computation continues through the various layers of the neural network, until it reaches a final layer. At this point, the output of the neural network routine can be read from the values in the final layer. Unlike fully-connected neural networks, convolutional neural networks operate by associating an array of values with each neuron, rather than a single value. The transformation of a neuron value for the subsequent layer is generalized from multiplication to convolution.

The machine learning procedure used according to some embodiments of the present invention is a trained machine learning procedure, which provides output that is related non-linearly to the parameters with which it is fed.

A machine learning procedure can be trained according to some embodiments of the present invention by feeding a machine learning training program with parameters that characterizes each of a cohort of subjects that has been diagnosed as either having or not having childhood obesity at obesity at age greater than the toddler age. Once the data are fed, the machine learning training program generates a trained machine learning procedure which can then be used without the need to re-train it.

For example, when it is desired to employ decision trees, machine learning training program learns the structure of each tree in a plurality of decision trees (e.g., how many nodes there are in each tree, and how these are connected to one another), and also selects the decision rules for split nodes of each tree. At least a portion of the decision rules relate to one or more of the parameters that characterize the subject. A simple decision rule may be a threshold for the value of a particular parameter, but more complex rules, relating to more than one parameter are also contemplated. The machine learning training program also accumulates data at the leaves of the trees. The structures of the trees, the decision rules for the split nodes, and the data at the leaves are all selected by the machine learning training program, automatically and typically without user intervention, such that the parameters at the root of the trees provide the likelihood for childhood obesity at the leaves of the trees. The final result of the machine learning training program in this case is a set of trees, where the structures, the decision rules for split nodes, and leaf data for each trees are defined by the machine learning training program.

The method proceeds to 13 at which the trained machine learning procedure is fed with the parameters, and to 14 at which an output indicative of the likelihood that the subject is expected to develop childhood obesity is received from the procedure. Preferably, the procedure provides the likelihood that the subject is expected to develop childhood obesity at an age greater than the toddler are, as further detailed hereinabove. In some embodiments of the present invention the method proceeds to 15 at which a report predating to the likelihood is generated. The report can be displayed on a display device or transmitted to a computer readable medium.

The method ends at 16.

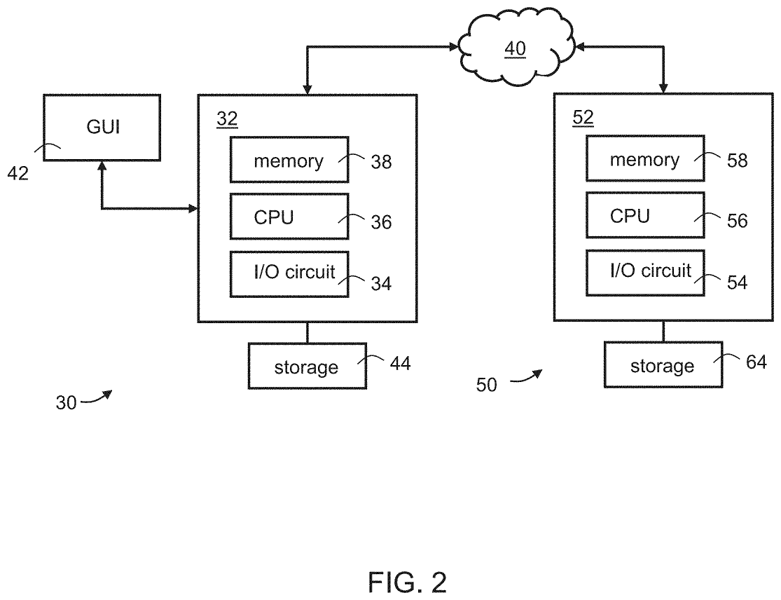

The prediction of likelihood for childhood obesity can be executed according to some embodiments of the present invention by a server-client configuration, as will now be explained with reference to FIG. 2.

FIG. 2 illustrates a client computer 30 having a hardware processor 32, which typically comprises an input/output (I/O) circuit 34, a hardware central processing unit (CPU) 36 (e.g., a hardware microprocessor), and a hardware memory 38 which typically includes both volatile memory and non-volatile memory. CPU 36 is in communication with I/O circuit 34 and memory 38. Client computer 30 preferably comprises a user interface, e.g., a graphical user interface (GUI), 42 in communication with processor 32. I/O circuit 34 preferably communicates information in appropriately structured form to and from GUI 42. Also shown is a server computer 50 which can similarly include a hardware processor 52, an I/O circuit 54, a hardware CPU 56, a hardware memory 58. I/O circuits 34 and 54 of client 30 and server 50 computers preferable operate as transceivers that communicate information with each other via a wired or wireless communication. For example, client 30 and server 50 computers can communicate via a network 40, such as a local area network (LAN), a wide area network (WAN) or the Internet. Server computer 50 can be in some embodiments be a part of a cloud computing resource of a cloud computing facility in communication with client computer 30 over the network 40.

GUI 42 and processor 32 can be integrated together within the same housing or they can be separate units communicating with each other. GUI 42 can optionally and preferably be part of a system including a dedicated CPU and I/O circuits (not shown) to allow GUI 42 to communicate with processor 32. Processor 32 issues to GUI 42 graphical and textual output generated by CPU 36. Processor 32 also receives from GUI 42 signals pertaining to control commands generated by GUI 42 in response to user input. GUI 42 can be of any type known in the art, such as, but not limited to, a keyboard and a display, a touch screen, and the like. In preferred embodiments, GUI 42 is a GUI of a mobile device such as a smartphone, a tablet, a smartwatch and the like. When GUI 42 is a GUI of a mobile device, the CPU circuit of the mobile device can serve as processor 32 and can execute the method optionally and preferably by executing code instructions.

Client 30 and server 50 computers can further comprise one or more computer-readable storage media 44, 64, respectively. Media 44 and 64 are preferably non-transitory storage media storing computer code instructions for executing the method of the present embodiments, and processors 32 and 52 execute these code instructions. The code instructions can be run by loading the respective code instructions into the respective execution memories 38 and 58 of the respective processors 32 and 52. Storage media 64 preferably also store one or more databases including a database of psychologically annotated olfactory perception signatures as further detailed hereinabove.

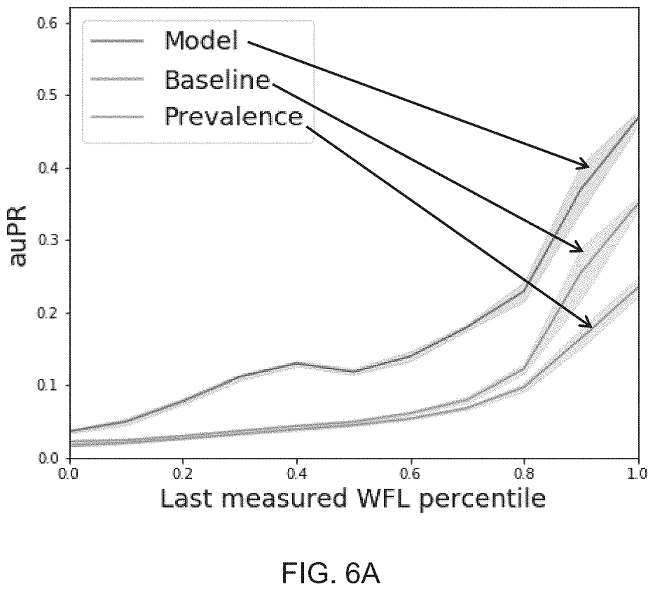

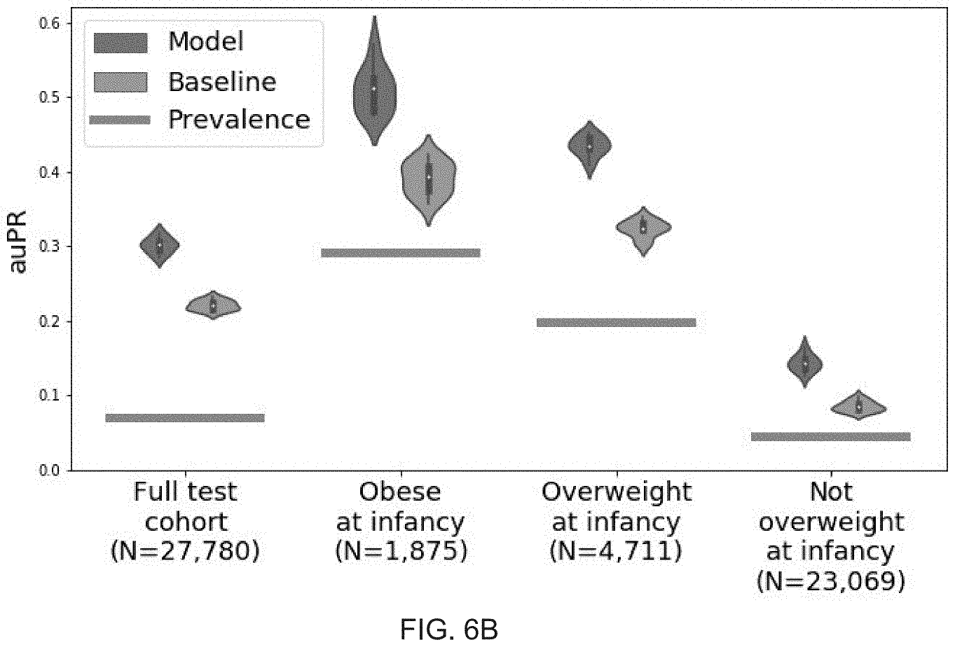

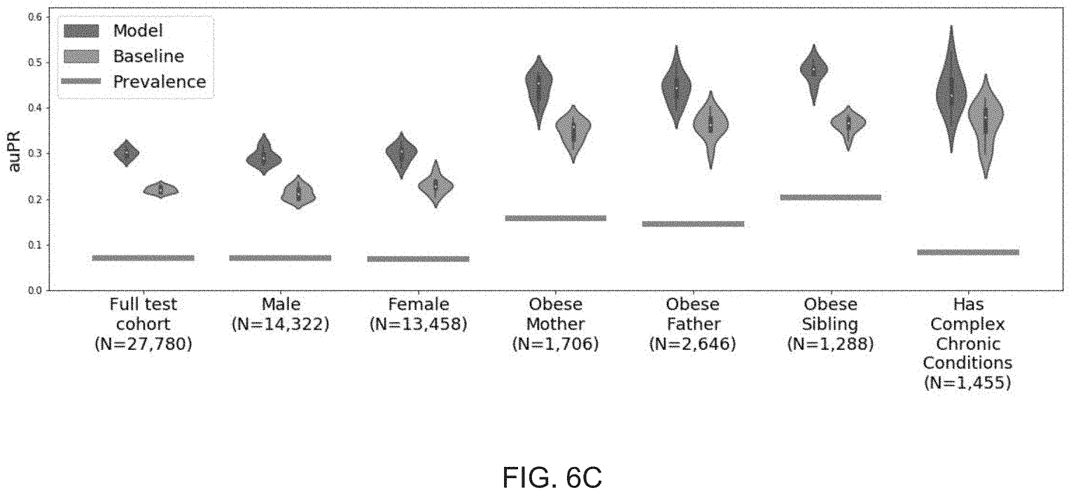

In operation, processor 32 of client computer 30 displays on GUI 42 a questionnaire and a set of questionnaire controls, such as, but not limited to, a slider, a dropdown menu, a combo box, a text box and the like. A representative example of a displayed questionnaire 60 and a set of controls 62 is shown in FIG. 6C. A person on behalf of the subject can enter response parameters using the questionnaire controls displayed on GUI 42.

Processor 32 receives the response parameters from GUI 42 and typically transmits these parameters to server computer 50 over network 40. Media 64 can store a machine learning procedure trained for predicting likelihoods for childhood obesity. Server computer 50 can access media 64, feed the stored procedure with the parameters received from client computer 30, and receive from the procedure an output indicative of the likelihood that the subject that is characterized by the parameters is expected to develop childhood obesity. Server computer 50 can also transmit to client computer 30 the obtained likelihood, and client computer 30 can display this information on GUI 42.

As used herein the term “about” refers to ±10%.

The word “exemplary” is used herein to mean “serving as an example, instance or illustration.” Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments.” Any particular embodiment of the invention may include a plurality of “optional” features unless such features conflict.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

Table 1.1 presents a list of 945 parameters from which parameters for feeing the machine learning procedure can be selected when the subject is an infant or toddler subject. The list is sorted according the significance of the respective feature for predicting the likelihood for childhood obesity, in descending order, so that from the standpoint of prediction accuracy it is more preferred to select a parameter that is listed higher in Table 1.1, than a parameter that is listed lower in Table 1.1. For example, when N parameters are used, it is preferred to select those parameters from lines 1 through M of Table 1.1, where N≤M≤945.

TABLE 1.1

| No. | Parameter |
|---|---|
| 1 | Last WFL zscore |
| 2 | Weight Routine checkup - 18-22 months |
| 3 | Weight Routine checkup - 12-16 months |
| 4 | WFL zscore median |
| 5 | Siblings median BMI zscore mean |
| 6 | WFL zscore mean |
| 7 | Weight Routine checkup - 4-6 months |
| 8 | Ethnicity: North Africa |
| 9 | Siblings mean BMI zscore mean |
| 10 | Siblings max BMI zscore mean |
| 11 | Father BMI median |
| 12 | WFL Routine checkup - 18-22 months |
| 13 | WFL zscore max |
| 14 | Father BMI max |
| 15 | Child mean Hb |
| 16 | Siblings at 5 years of age BMI zscore mean |
| 17 | Siblings min BMI zscore mean |
| 18 | Father BMI mean |
| 19 | Child mean Mpv |
| 20 | Father BMI min |
| 21 | Mother Pre-Pregnancy BMI max |
| 22 | Child All Antibiotics prescription day counts |
| 23 | Weight Routine checkup - 9-12 months |
| 24 | Mother Pre-Pregnancy BMI median |
| 25 | Child diagnosed Acute upper respiratory infections of multiple or |
| | unsp.sites |
| 26 | Mother 24-40 weeks MCV |
| 27 | Height Routine checkup - 12-16 months |
| 28 | Mother Pre-Pregnancy BMI mean |
| 29 | Child mean Baso % |
| 30 | Mother 24-40 weeks MCH |
| 31 | Child mean Rdw |
| 32 | Child mean Plt |
| 33 | Child count Salbutamol |
| 34 | Height Routine checkup - 18-22 months |
| 35 | Weight Routine checkup - 6-9 months |
| 36 | Age of Father at birth |
| 37 | Child mean Eosinophils abs-dif |
| 38 | Siblings count BMI zscore std |
| 39 | Mother Pre-Pregnancy BMI min |
| 40 | WFL Routine checkup - 1-2 months |
| 41 | Ethnicity: Ethiopia |
| 42 | Weight Routine checkup - 2-3 months |
| 43 | Child mean Mcv |
| 44 | Child count Bethamethasone |
| 45 | Mother last BMI 24-40 weeks |
| 46 | Age of Mother at birth |
| 47 | WFL Routine checkup - 9-12 months |
| 48 | WFL zscore slope |
| 49 | Father Weight median |
| 50 | WFL Routine checkup - 12-16 months |
| 51 | Locality type: Jewish Locality 100,000-199,999 residents |
| 52 | Age at last WFL |
| 53 | Mother Pre-Pregnancy Weight max |
| 54 | Ethnicity: Unknown |
| 55 | Weight Routine checkup - 1-2 months |
| 56 | Mother last BMI 0-12 weeks |
| 57 | WFL zscore intercept |
| 58 | Height Routine checkup - 4-6 months |
| 59 | Child diagnosed Nausea and vomiting |
| 60 | Ethnicity: North America |
| 61 | Father Height median |
| 62 | Height Routine checkup - 6-9 months |
| 63 | Mother Pre-Pregnancy Weight mean |
| 64 | Ethnicity: West Europe |
| 65 | Child mean Hct |
| 66 | Locality type: Non-Jewish Locality 5,000-9,999 residents |
| 67 | Child mean Ggt |
| 68 | Mother 12-24 weeks VITAMIN B12 |
| 69 | Child diagnosed Dyspnea and respiratory abnormalities |
| 70 | Mother 0-12 weeks MCH |
| 71 | Child mean Mch |
| 72 | Father std Cholesterol |
| 73 | Child mean Wbc |
| 74 | Child diagnosed Colitis, enteritis, gastroenteritis presumed |
| | infectious origin |
| 75 | Child diagnosed Acute upper respiratory infections of unspecified |
| | site |
| 76 | Mother Pre-Pregnancy Weight median |
| 77 | Siblings min BMI zscore std |
| 78 | Child mean Protein-total |
| 79 | Week of year born |
| 80 | Child mean Hypo % |
| 81 | Mother Pre-Pregnancy Weight min |
| 82 | WFL zscore min |
| 83 | Child diagnosed Hypertrophy of tonsils and adenoids |
| 84 | Mother Pre-pregnancy CMV IgG |
| 85 | Mother Pre-pregnancy PDW |
| 86 | Child diagnosed Acute tonsillitis |
| 87 | Mother 24-40 weeks GLUCOSE 50 g |
| 88 | Mother Pre-pregnancy GGT |
| 89 | Child mean Gpt (alt) |
| 90 | Child mean Albumin |
| 91 | Child diagnosed Fever |
| 92 | Child mean Ferritin |
| 93 | Father Height mean |
| 94 | Height Routine checkup - 9-12 months |
| 95 | Ethnicity: Iraq |
| 96 | Siblings mean BMI zscore std |
| 97 | Child count Budesonide |
| 98 | Father max Triglycerides |
| 99 | Mother 12-24 weeks RBC |
| 100 | Mother 0-12 weeks WBC |
| 101 | Siblings std BMI zscore mean |
| 102 | Mother last Diastolic Blood Pressure 24-40 weeks |
| 103 | Mother 12-24 weeks HB |
| 104 | Mother 12-24 weeks LUC % |
| 105 | Child Penicillin Antibiotics prescription day counts |
| 106 | Child mean Ldh |
| 107 | Mother 0-12 weeks VITAMIN B12 |
| 108 | Child diagnosed Lack of coordination |
| 109 | Mother 0-12 weeks HCT |
| 110 | Mother Pre-pregnancy GLUCOSE 50 g |
| 111 | Father mean Cholesterol- hdl |
| 112 | Father mean Triglycerides |
| 113 | Father Height min |
| 114 | Child mean Tsh |
| 115 | Siblings count BMI zscore mean |
| 116 | Mother 0-12 weeks LYMP.abs |
| 117 | Child mean Rdw-cv |
| 118 | WFL Routine checkup - 6-9 months |
| 119 | Locality type: Non-Jewish Locality 10,000-19,999 residents |
| 120 | Mother Pre-pregnancy GLUCOSE |
| 121 | Child diagnosed Acute bronchiolitis |
| 122 | Mother last BMI 12-24 weeks |
| 123 | Father std Glucose |
| 124 | Mother Pre-pregnancy CK—CREAT.KINASE(CPK) |
| 125 | Child mean Creatinine |
| 126 | Father std Cholesterol-ldl calc |
| 127 | Father min Cholesterol- hdl |
| 128 | Mother last BMI Pre-pregnancy |
| 129 | Mother Pre-pregnancy TSH |
| 130 | Date of Birth |
| 131 | Mother last Weight Pre-pregnancy |
| 132 | Mother Pre-pregnancy MCHC |
| 133 | Mother Pre-pregnancy LYMP.abs |
| 134 | Siblings median BMI zscore std |
| 135 | Mother 12-24 weeks IRON |
| 136 | Mother count Roxithromycin |
| 137 | Mother last Weight 12-24 weeks |
| 138 | Mother 24-40 weeks MPV |
| 139 | Mother 12-24 weeks GLUCOSE |
| 140 | Mother Pre-pregnancy PT % |
| 141 | Height Routine checkup - 2-3 months |
| 142 | Mother 24-40 weeks VITAMIN B12 |
| 143 | Father max Glucose |
| 144 | Father Weight max |
| 145 | Mother 24-40 weeks EOS % |
| 146 | Child diagnosed Cough |
| 147 | Child count Amoxicillin |
| 148 | Mother 24-40 weeks GLUCOSE (GTT) 0′ |
| 149 | Mother Pre-pregnancy HCT |
| 150 | Mother Pre-pregnancy BILIRUBIN-DIRECT |
| 151 | Age at Target measurement |
| 152 | Mother 0-12 weeks MPV |
| 153 | Ethnicity: East Europe |
| 154 | Siblings max BMI zscore std |
| 155 | Child mean Glucose |
| 156 | Child mean Stabs %-dif |
| 157 | Height Routine checkup - 1-2 months |
| 158 | Father mean Glucose |
| 159 | Child mean Mono % |
| 160 | Mother 0-12 weeks NEUT.abs |
| 161 | Child mean Neutrophils abs-dif |
| 162 | Father Weight mean |
| 163 | Mother Pre-pregnancy T4- FREE |
| 164 | WFL zscore slope_std_err |
| 165 | Mother 24-40 weeks RBC |
| 166 | Mother Pre-pregnancy LYM % |
| 167 | Child diagnosed Hearing loss |
| 168 | Child mean Eos.abs |
| 169 | Child mean Sodium |
| 170 | Mother 24-40 weeks ALK. PHOSPHATASE |
| 171 | Child diagnosed Urinary tract infection, site not specified |
| 172 | Child mean Luc abs |
| 173 | Mother 0-12 weeks EOS.abs |
| 174 | Father min Triglycerides |
| 175 | Mother 0-12 weeks MONO.abs |
| 176 | Child mean Luc % |
| 177 | Mother Pre-pregnancy MPV |
| 178 | Mother Pre-pregnancy NEUT % |
| 179 | Mother 24-40 weeks APTT-R |
| 180 | Child diagnosed Otorrhea |
| 181 | Siblings at 13 years of age BMI zscore mean |
| 182 | Ethnicity: Muslim Arab |
| 183 | Child mean Atypical lymph.%-dif |
| 184 | Mother Pre-pregnancy PHOSPHORUS |
| 185 | WFL Routine checkup - 2-3 months |
| 186 | Father count Metformin |
| 187 | WFL zscore count |
| 188 | Child mean T4- free |
| 189 | Mother Pre-pregnancy NEUT.abs |
| 190 | Mother 12-24 weeks MCHC |
| 191 | Child mean Chloride |
| 192 | Mother 24-40 weeks HEMOGLOBIN A1C % |
| 193 | Mother Pre-pregnancy CHOLESTEROL-LDL calc |
| 194 | Child mean Lym % |
| 195 | Child mean Mono.abs |
| 196 | Child diagnosed Sleep disturbances |
| 197 | Child mean Micro % |
| 198 | Child mean Calcium |
| 199 | Child mean Rbc |
| 200 | Mother last Systolic Blood Pressure 0-12 weeks |
| 201 | Child mean Lymphocytes abs-dif |
| 202 | WFL Routine checkup - 4-6 months |
| 203 | Father median Triglycerides |
| 204 | Mother 24-40 weeks MICRO % |
| 205 | Mother last Systolic Blood Pressure 12-24 weeks |
| 206 | Mother 24-40 weeks MONO.abs |
| 207 | Mother 12-24 weeks PLT |
| 208 | Locality type: Jewish Locality 10,000-19,999 residents |
| 209 | Child mean Alk. phosphatase |
| 210 | Child mean Baso abs |
| 211 | Child mean Eos % |
| 212 | Mother Pre-pregnancy LDH |
| 213 | Child mean Atypical lymph-dif |
| 214 | Mother 0-12 weeks HEPATITIS Bs Ab |
| 215 | Child mean Hyper % |
| 216 | Child mean Got (ast) |
| 217 | Mother Pre-pregnancy PLT |
| 218 | Father min Glucose |
| 219 | Child mean Lymp.abs |
| 220 | Father max Non-hdl_cholesterol |
| 221 | Mother 12-24 weeks NEUT % |
| 222 | Mother 24-40 weeks HYPO % |
| 223 | Mother last Systolic Blood Pressure Pre-pregnancy |
| 224 | Father Height max |
| 225 | Mother last Systolic Blood Pressure 24-40 weeks |
| 226 | Father median Cholesterol- hdl |
| 227 | Mother 12-24 weeks T4- FREE |
| 228 | Mother Pre-pregnancy UREA |
| 229 | Mother Pre-pregnancy MAGNESIUM |
| 230 | Mother 0-12 weeks CHOLESTEROL/HDL |
| 231 | Child mean Mchc |
| 232 | Mother 24-40 weeks LYM % |
| 233 | Mother 12-24 weeks MCV |
| 234 | Mother Pre-pregnancy MONO.abs |
| 235 | Child mean Neut.abs |
| 236 | Mother Pre-pregnancy WBC |
| 237 | Mother 12-24 weeks MONO.abs |
| 238 | Mother 24-40 weeks HCT |
| 239 | Mother 0-12 weeks CMV IgG |
| 240 | Mother 24-40 weeks PLT |
| 241 | WFL zscore std |
| 242 | Birth weight |
| 243 | Mother Pre-pregnancy PROTEIN-TOTAL |
| 244 | Mother 12-24 weeks CMV IgG |
| 245 | Child mean Cholesterol |
| 246 | Mother 24-40 weeks CMV IgG |
| 247 | Mother 0-12 weeks SODIUM |
| 248 | Mother 24-40 weeks NEUT % |
| 249 | Mother 24-40 weeks MCHC |
| 250 | Father Weight min |
| 251 | Mother count Amoxicillin |
| 252 | Father mean Cholesterol |
| 253 | Child mean Bilirubin total |
| 254 | Father median Glucose |
| 255 | Child mean Pdw |
| 256 | Mother Pre-pregnancy CHOLESTEROL |
| 257 | Child Macrolides Antibiotics prescription day counts |
| 258 | Mother 0-12 weeks MONO % |
| 259 | Mother 24-40 weeks LYMP.abs |
| 260 | Mother 12-24 weeks NEUT.abs |
| 261 | Mother Pre-pregnancy HYPER % |
| 262 | Child mean Iron |
| 263 | Mother 12-24 weeks TSH |
| 264 | Mother count Cabergoline |
| 265 | Mother last Weight 0-12 weeks |
| 266 | Mother Pre-pregnancy PCT |
| 267 | Father Height std |
| 268 | Mother 0-12 weeks TRIGLYCERIDES |
| 269 | Mother 0-12 weeks GLUCOSE |
| 270 | Father std Cholesterol/hdl |
| 271 | Mother Pre-pregnancy HYPO % |
| 272 | Mother 24-40 weeks FERRITIN |
| 273 | Child count Terbutaline |
| 274 | Child mean Monocytes %-dif |
| 275 | Jewish Locality |
| 276 | Child mean Uric acid |
| 277 | Child diagnosed Acute nonsuppurative otitis media |
| 278 | Father BMI std |
| 279 | Mother Pre-pregnancy BASO % |
| 280 | Mother 24-40 weeks SODIUM |
| 281 | Mother Pre-pregnancy VITAMIN B12 |
| 282 | Mother 0-12 weeks ESTRADIOL (E-2) |
| 283 | Mother 0-12 weeks LYM % |
| 284 | Mother 12-24 weeks EOS % |
| 285 | Mother 24-40 weeks NEUT.abs |
| 286 | Mother 24-40 weeks NEUTROPHILS abs-DIF |
| 287 | Father diagnosed Diabetes mellitus |
| 288 | Mother Pre-pregnancy CREATININE |
| 289 | Child Cephalosporin Antibiotics prescription day counts |
| 290 | Father Weight std |
| 291 | Mother 24-40 weeks HB |
| 292 | Mother BMI delta 12-24 weeks to 24-40 weeks |
| 293 | Mother 0-12 weeks GGT |
| 294 | Child mean Urea |
| 295 | Mother 0-12 weeks LH |
| 296 | Mother 24-40 weeks RDW |
| 297 | Mother 12-24 weeks HbA2 |
| 298 | Mother 0-12 weeks MCV |
| 299 | Mother Pre-pregnancy MONO % |
| 300 | Mother Pre-pregnancy HB |
| 301 | Child mean Micro %/hypo % |
| 302 | Mother 24-40 weeks LUC % |
| 303 | Mother count Enoxaparin |
| 304 | Child mean Monocytes abs-dif |
| 305 | Mother 24-40 weeks MONO % |
| 306 | Mother 0-12 weeks NEUT % |
| 307 | Mother 24-40 weeks WBC |
| 308 | Child diagnosed Acute conjunctivitis |
| 309 | Father mean Non-hdl_cholesterol |
| 310 | Child mean Neutrophils %-dif |
| 311 | Mother 0-12 weeks EOS % |
| 312 | Mother 0-12 weeks RDW |
| 313 | Mother Pre-pregnancy RDW |
| 314 | Mother 12-24 weeks LYM % |
| 315 | Mother Pre-pregnancy SHBG |
| 316 | Mother Pre-pregnancy FOLIC ACID |
| 317 | Child mean Transferrin |
| 318 | Child diagnosed Other viral diseases; nos |
| 319 | Mother 0-12 weeks HYPO % |
| 320 | Mother Pre-pregnancy MICRO % |
| 321 | Mother 24-40 weeks BILIRUBIN TOTAL |
| 322 | Child mean Lymphocytes %-dif |
| 323 | Mother Pre-pregnancy SODIUM |
| 324 | Mother Pre-pregnancy RBC |
| 325 | Child diagnosed Teething syndrome |
| 326 | Child count Prednisolone |
| 327 | Mother 24-40 weeks BASO % |
| 328 | Mother 24-40 weeks LYMPHOCYTES abs-DIF |
| 329 | Mother 0-12 weeks PROGESTERONE |
| 330 | Father BMI count |
| 331 | Mother Pre-pregnancy TRIGLYCERIDES |
| 332 | Father max Cholesterol |
| 333 | Mother 12-24 weeks LYMP.abs |
| 334 | Child diagnosed Benign neoplasm of skin, site unspecified |
| 335 | Mother last Diastolic Blood Pressure 0-12 weeks |
| 336 | Mother Pre-pregnancy GLOBULIN |
| 337 | Mother 24-40 weeks CREATININE |
| 338 | Father max Cholesterol-ldl calc |
| 339 | Father max Cholesterol- hdl |
| 340 | Mother Pre-pregnancy ESR |
| 341 | Mother 12-24 weeks PT-SEC |
| 342 | Mother 24-40 weeks LUC abs |
| 343 | Mother 24-40 weeks MPXI |
| 344 | Mother Pre-Pregnancy BMI std |
| 345 | Mother 12-24 weeks FERRITIN |
| 346 | Mother 0-12 weeks MPXI |
| 347 | Mother 0-12 weeks TSH |
| 348 | Mother 24-40 weeks GOT (AST) |
| 349 | Mother 24-40 weeks HYPER % |
| 350 | Mother 24-40 weeks EOSINOPHILS abs-DIF |
| 351 | Mother 12-24 weeks WBC |
| 352 | Father mean Cholesterol-ldl calc |
| 353 | Ethnicity: Iran |
| 354 | Child count Dimethindene |
| 355 | Father std Triglycerides |
| 356 | Mother Pre-pregnancy HDW |
| 357 | Mother 0-12 weeks UREA |
| 358 | Mother 12-24 weeks HCT |
| 359 | Mother Pre-pregnancy HEPATITIS Bs Ab |
| 360 | Child mean Triglycerides |
| 361 | Child diagnosed Acute lymphadenitis |
| 362 | Mother 0-12 weeks LDH |
| 363 | Mother 12-24 weeks POTASSIUM |
| 364 | Child mean Neut % |
| 365 | Child diagnosed Unspecified fetal and neonatal jaundice |
| 366 | Mother Pre-Pregnancy Weight std |
| 367 | Mother 12-24 weeks MICRO % |
| 368 | Mother Pre-pregnancy BILIRUBIN TOTAL |
| 369 | Mother 0-12 weeks HB |
| 370 | Child mean Mpxi |
| 371 | Mother Pre-pregnancy C-REACTIVE PROTEIN |
| 372 | Mother Pre-pregnancy MCV |
| 373 | Mother Pre-pregnancy DHEA SULPHATE |
| 374 | Child mean Pct |
| 375 | Father min Cholesterol |
| 376 | Locality type: Jewish Locality 50,000-99,999 residents |
| 377 | Mother Pre-pregnancy EOS % |
| 378 | Father median Cholesterol |
| 379 | Child mean Hct/hgb ratio |
| 380 | Mother 24-40 weeks BILIRUBIN-DIRECT |
| 381 | Child diagnosed Diaper or napkin rash |
| 382 | Mother 24-40 weeks STABS %-DIF |
| 383 | Child mean Stabs abs-dif |
| 384 | Siblings at 5 years of age BMI zscore std |
| 385 | Child diagnosed Congenital anomalies of lower limb, including |
| | pelvic girdle |
| 386 | Father std Cholesterol- hdl |
| 387 | Child count Cefalexin |
| 388 | Mother 12-24 weeks HYPO % |
| 389 | Child diagnosed Oral aphthae |
| 390 | Mother 24-40 weeks STABS abs-DIF |
| 391 | Child mean Phosphorus |
| 392 | Mother 0-12 weeks LUC % |
| 393 | Mother 12-24 weeks SODIUM |
| 394 | Mother 24-40 weeks GLUCOSE (GTT) 60′ |
| 395 | Mother 24-40 weeks CHOLESTEROL |
| 396 | Child count Erythromycin |
| 397 | No. of Siblings with BMI data |
| 398 | Mother 12-24 weeks CREATININE |
| 399 | Mother 24-40 weeks GLUCOSE (GTT) 180′ |
| 400 | Mother 12-24 weeks EOS.abs |
| 401 | Child diagnosed Asthma |
| 402 | Mother Pre-pregnancy COMPLEMENT C3 |
| 403 | Mother Pre-pregnancy EOS.abs |
| 404 | Ethnicity: Asian |
| 405 | Mother 24-40 weeks T3- FREE |
| 406 | Mother Pre-pregnancy FERRITIN |
| 407 | Mother Pre-pregnancy AMYLASE |
| 408 | Father count Pravastatin |
| 409 | Mother 24-40 weeks MONOCYTES abs-DIF |
| 410 | Mother 24-40 weeks GPT (ALT) |
| 411 | Mother Pre-pregnancy URIC ACID |
| 412 | Father diagnosed Obesity, unspecified |
| 413 | Mother 24-40 weeks NEUTROPHILS %-DIF |
| 414 | Child diagnosed Bronchopneumonia, organism unspecified |
| 415 | Mother 0-12 weeks MCHC |
| 416 | Mother 12-24 weeks MONO % |
| 417 | Mother Pre-pregnancy FIBRINOGEN CALCU |
| 418 | Mother Pre-pregnancy MPXI |
| 419 | Child Beta lactam Penicillin Antibiotics prescription day counts |
| 420 | Mother 0-12 weeks URIC ACID |
| 421 | Mother Pre-pregnancy LH |
| 422 | Mother 24-40 weeks MACRO % |
| 423 | Mother Pre-pregnancy MCH |
| 424 | Mother 24-40 weeks BASO abs |
| 425 | Father count Cholesterol-ldl calc |
| 426 | Mother 0-12 weeks MICRO % |
| 427 | Mother Weight delta Pre-pregnancy to 0-12 weeks |
| 428 | Child diagnosed Constipation |
| 429 | Siblings std BMI zscore std |
| 430 | Mother 24-40 weeks LDH |
| 431 | Mother 0-12 weeks PLT |
| 432 | Siblings at 13 years of age BMI zscore std |
| 433 | Father count Glucose |
| 434 | Mother Pre-pregnancy BILIRUBIN INDIRECT |
| 435 | Child mean Eosinophils %-dif |
| 436 | Mother 24-40 weeks URIC ACID |
| 437 | Mother BMI delta Pre-pregnancy to 0-12 weeks |
| 438 | Mother 12-24 weeks GGT |
| 439 | Mother 0-12 weeks GPT (ALT) |
| 440 | Mother 0-12 weeks PHOSPHORUS |
| 441 | Mother Pre-pregnancy LUC % |
| 442 | Child diagnosed U.r.i. (head cold) |
| 443 | Mother 0-12 weeks HYPER % |
| 444 | Mother 0-12 weeks CREATININE |
| 445 | Mother 12-24 weeks MICRO %/HYPO % |
| 446 | Mother 0-12 weeks MACRO % |
| 447 | Mother 12-24 weeks RDW |
| 448 | Mother Pre-pregnancy POTASSIUM |
| 449 | Mother 0-12 weeks RBC |
| 450 | Mother Pre-pregnancy ALK. PHOSPHATASE |
| 451 | Child diagnosed Enlargement of lymph nodes |
| 452 | Mother Pre-pregnancy ALBUMIN |
| 453 | Mother 12-24 weeks TRIGLYCERIDES |
| 454 | Mother 0-12 weeks AMYLASE |
| 455 | Father min Cholesterol-ldl calc |
| 456 | Mother 0-12 weeks ALK. PHOSPHATASE |
| 457 | Mother Pre-pregnancy PT-SEC |
| 458 | Child diagnosed Diarrhea |
| 459 | Mother 0-12 weeks VITAMIN D (25-OH) |
| 460 | Child diagnosed Pneumonia |
| 461 | Mother 12-24 weeks MCH |
| 462 | Child mean Potassium |
| 463 | Mother Pre-pregnancy CALCIUM |
| 464 | Father count Cholesterol- hdl |
| 465 | Father median Cholesterol-ldl calc |
| 466 | Mother Pre-pregnancy COMPLEMENT C4 |
| 467 | Mother count Ofloxacin |
| 468 | Child mean C-reactive protein |
| 469 | Mother last Weight 24-40 weeks |
| 470 | Mother 0-12 weeks CHOLESTEROL-LDL calc |
| 471 | Mother Pre-pregnancy MACRO % |
| 472 | Mother count Phenoxymethylpenicillin |
| 473 | Mother 0-12 weeks HDW |
| 474 | Mother 24-40 weeks TRIGLYCERIDES |
| 475 | Mother Pre-pregnancy TESTOSTERONE- TOTAL |
| 476 | Father std Non-hdl_cholesterol |
| 477 | Child diagnosed Contusion of unspecified site |
| 478 | Mother 0-12 weeks NON-HDL_CHOLESTEROL |
| 479 | Child diagnosed Esophagitis |
| 480 | Child mean Macro % |
| 481 | Mother last Diastolic Blood Pressure Pre-pregnancy |
| 482 | Mother 0-12 weeks APTT-sec |
| 483 | Child count Cefuroxime |
| 484 | Child diagnosed Atopic dermatitis/eczema |
| 485 | Mother 24-40 weeks MICRO %/HYPO % |
| 486 | Ethnicity: USSR |
| 487 | Mother 12-24 weeks MPXI |
| 488 | Mother 0-12 weeks BASO % |
| 489 | Father min Non-hdl_cholesterol |
| 490 | Mother Pre-pregnancy NON-HDL_CHOLESTEROL |
| 491 | Mother 0-12 weeks GLOBULIN |
| 492 | Mother 12-24 weeks MACRO % |
| 493 | Child diagnosed Stridor |
| 494 | Father count Simvastatin |
| 495 | Mother 12-24 weeks LUC abs |
| 496 | Child diagnosed Infectious diarrhea |
| 497 | Mother 12-24 weeks PT-INR |
| 498 | Mother 0-12 weeks GOT (AST) |
| 499 | Father min Cholesterol/hdl |
| 500 | Mother 24-40 weeks GLUCOSE |
| 501 | Mother 24-40 weeks EOS.abs |
| 502 | Child diagnosed Chronic rhinitis |
| 503 | Mother 12-24 weeks UREA |
| 504 | Mother 0-12 weeks PROTEIN-TOTAL |
| 505 | Mother Pre-pregnancy ALY |
| 506 | Mother Pre-pregnancy FREE ANDROGEN INDEX |
| 507 | Child diagnosed Unsp.viral infect.in conditions classif.elsewhere, |
| | unsp.site |
| 508 | Mother 0-12 weeks POTASSIUM |
| 509 | Mother 12-24 weeks AMYLASE |
| 510 | Mother 12-24 weeks CK—CREAT.KINASE(CPK) |
| 511 | Mother Pre-pregnancy GPT (ALT) |
| 512 | Mother 0-12 weeks CHOLESTEROL |
| 513 | Mother 12-24 weeks BASO % |
| 514 | Child diagnosed Anorexia |
| 515 | Mother Pre-pregnancy CORTISOL-BLOOD |
| 516 | Mother 24-40 weeks RDW-CV |
| 517 | Mother Pre-pregnancy ESTRADIOL (E-2) |
| 518 | Mother 12-24 weeks MPV |
| 519 | Child diagnosed Other specified disease of white blood cells |
| 520 | Mother Pre-pregnancy PROLACTIN |
| 521 | Mother 24-40 weeks TSH |
| 522 | is Male |
| 523 | Child diagnosed Lack of expected normal physiological |
| | development |
| 524 | Mother 0-12 weeks CK—CREAT.KINASE(CPK) |
| 525 | Father median Non-hdl_cholesterol |
| 526 | Father mean Cholesterol/hdl |
| 527 | Mother 0-12 weeks FOLIC ACID |
| 528 | Mother 24-40 weeks IRON |
| 529 | Mother 0-12 weeks LUC abs |
| 530 | Mother Pre-pregnancy RUBELLA Ab IgG |
| 531 | Mother 0-12 weeks ALBUMIN |
| 532 | Child mean Bilirubin-direct |
| 533 | Mother 0-12 weeks IRON |
| 534 | Mother 0-12 weeks RUBELLA Ab IgG |
| 535 | Mother 24-40 weeks AMYLASE |
| 536 | Number of twin siblings |
| 537 | Mother Pre-pregnancy ANDROSTENEDIONE |
| 538 | Father count Enalapril |
| 539 | Mother count Mebendazole |
| 540 | Mother 24-40 weeks CHLORIDE |
| 541 | Child diagnosed Influenza |
| 542 | Child count Desloratadine |
| 543 | Mother 24-40 weeks HDW |
| 544 | Child count Ketotifen |
| 545 | Child diagnosed Dermatitis due to food taken internally |
| 546 | Mother 24-40 weeks GLUCOSE (GTT) 120′ |
| 547 | Father count Cholesterol |
| 548 | Mother 12-24 weeks PCT |
| 549 | Mother 24-40 weeks UREA |
| 550 | Child count Ipratropium bromide |
| 551 | Child diagnosed Acute pharyngitis |
| 552 | Child diagnosed Acute suppurative otitis media |
| 553 | Mother 0-12 weeks TOXOPLASMA IgG |
| 554 | Mother Pre-pregnancy MICRO %/HYPO % |
| 555 | Mother 24-40 weeks PROTEIN-TOTAL |
| 556 | Mother 12-24 weeks TOXOPLASMA IgG |
| 557 | Mother 0-12 weeks FSH |
| 558 | Father count Non-hdl_cholesterol |
| 559 | Child diagnosed Acute nasopharyngitis (common cold) |
| 560 | Mother 24-40 weeks CHOLESTEROL- HDL |
| 561 | Mother 24-40 weeks PT-SEC |
| 562 | Mother Pre-pregnancy ANTI CARDIOLIPIN IgG |
| 563 | Mother Pre-Pregnancy BMI count |
| 564 | Mother 24-40 weeks PDW |
| 565 | Mother 24-40 weeks MONOCYTES %-DIF |
| 566 | Mother 0-12 weeks MICRO %/HYPO % |
| 567 | Mother Pre-pregnancy TRANSFERRIN |
| 568 | Mother Pre-pregnancy GOT (AST) |
| 569 | Child diagnosed Other diseases of conjunctiva due to viruses and |
| | chlamydiae |
| 570 | Mother Pre-pregnancy PT-INR |
| 571 | Mother 24-40 weeks CALCIUM |
| 572 | Child diagnosed Other atopic dermatitis and related conditions |
| 573 | Mother 0-12 weeks HEMOGLOBIN A |
| 574 | Mother Pre-pregnancy LUC abs |
| 575 | Father count Amlodipine |
| 576 | Mother 12-24 weeks ALK. PHOSPHATASE |
| 577 | Father count Triglycerides |
| 578 | Mother 0-12 weeks CALCIUM |
| 579 | Child count Azithromycin |
| 580 | Mother 12-24 weeks FOLIC ACID |
| 581 | Mother Pre-pregnancy FSH |
| 582 | Child diagnosed Pneumonia, organism unspecified |
| 583 | Mother Pre-pregnancy CHOLESTEROL- HDL |
| 584 | Locality type: Non-Jewish Other Rural Locality |
| 585 | Child count Ahiston drop cd |
| 586 | Mother Pre-pregnancy PROGESTERONE |
| 587 | Mother 0-12 weeks T4- FREE |
| 588 | Mother 12-24 weeks BASO abs |
| 589 | Child diagnosed Other and unspec.noninfectious gastroenteritis |
| | and colitis |
| 590 | Child diagnosed Asthma, unspecified |
| 591 | Mother Pre-pregnancy ANTITHROMBIN-III |
| 592 | Mother 24-40 weeks TOXOPLASMA IgG |
| 593 | Mother 0-12 weeks PT-SEC |
| 594 | Child diagnosed Volume depletion disorder |
| 595 | Mother Pre-pregnancy CONTROL PTT |
| 596 | Mother 24-40 weeks EOSINOPHILS %-DIF |
| 597 | Mother Pre-pregnancy 17-OH-PROGESTERONE |
| 598 | Father count Cholesterol/hdl |
| 599 | Mother Pre-pregnancy IRON |
| 600 | Mother Pre-pregnancy HEMOGLOBIN A1C % |
| 601 | Mother 12-24 weeks HYPER % |
| 602 | Mother 0-12 weeks BASO abs |
| 603 | Locality type: Non-Jewish Locality 2,000-4,999 residents |
| 604 | Mother Pre-pregnancy APTT-sec |
| 605 | Mother count Fluticasone |
| 606 | Mother 24-40 weeks HCT/HGB Ratio |
| 607 | Father count Bezafibrate |
| 608 | Locality type: Jewish Locality 200,000-499,999 residents |
| 609 | Father diagnosed Obesity (bmi >30) |
| 610 | Mother count Omeprazole |
| 611 | Child count Co-amoxiclav cd |
| 612 | Mother 24-40 weeks PT-INR |
| 613 | Mother Pre-pregnancy HCT/HGB Ratio |
| 614 | Child count Montelukast |
| 615 | Child diagnosed Infectious colitis, enteritis, and gastroenteritis |
| 616 | Mother Pre-Pregnancy Weight count |
| 617 | Mother count Estradiol |
| 618 | Mother 24-40 weeks PCT |
| 619 | Mother Pre-pregnancy T3-TOTAL |
| 620 | Mother count Follitropin alfa |
| 621 | Child diagnosed Acute bronchitis |
| 622 | Ethnicity: Yemen |
| 623 | Child diagnosed Abdominal pain |
| 624 | Child diagnosed Other and unspecified injury to unspecified site |
| 625 | Child count Prothiazine/promethazine expectorant cd |
| 626 | Mother 24-40 weeks PT % |
| 627 | Locality type: Moshav |
| 628 | Mother Pre-pregnancy VLDL |
| 629 | Mother 24-40 weeks POTASSIUM |
| 630 | Child count Co-trimoxazole cd |
| 631 | Mother 12-24 weeks HbF |
| 632 | Mother 24-40 weeks BILIRUBIN INDIRECT |
| 633 | Mother 24-40 weeks GLOM.FILTR.RATE |
| 634 | Mother 24-40 weeks PHOSPHORUS |
| 635 | Father max Cholesterol/hdl |
| 636 | Child diagnosed Iron deficiency anemia, unspecified |
| 637 | Mother Pre-pregnancy ALY % |
| 638 | Child diagnosed Rash and other nonspecific skin eruption |
| 639 | Mother 0-12 weeks PT % |
| 640 | Mother 12-24 weeks PT % |
| 641 | Mother 24-40 weeks TRANSFERRIN |
| 642 | Father Weight count |
| 643 | Child diagnosed Late effect of injury to cranial nerve |
| 644 | Mother Pre-pregnancy T3- FREE |
| 645 | Mother 12-24 weeks PROTEIN-TOTAL |
| 646 | Cesarean birth |
| 647 | Mother Pre-pregnancy BASO abs |
| 648 | Mother 0-12 weeks T3- FREE |
| 649 | Mother Pre-pregnancy RDW-CV |
| 650 | Mother count Levothyroxine sodium |
| 651 | Child Sulfonamides Antibiotics prescription day counts |
| 652 | Mother 12-24 weeks ALBUMIN |
| 653 | Child diagnosed Undescended testicle |
| 654 | Mother 12-24 weeks CHOLESTEROL |
| 655 | Child diagnosed Hearing complaints |
| 656 | Mother 24-40 weeks MAGNESIUM |
| 657 | Mother 0-12 weeks PDW |
| 658 | Mother 0-12 weeks TRANSFERRIN |
| 659 | Mother 24-40 weeks HbA2 |
| 660 | Mother 12-24 weeks T3- FREE |
| 661 | Mother count Aspirin |
| 662 | Mother 0-12 weeks BLOOD TYPE |
| 663 | Mother count Human menopausal gonadotrophin |
| 664 | Mother count Co-amoxiclav cd |
| 665 | Mother 24-40 weeks T4- FREE |
| 666 | Child diagnosed Contact dermatitis and other eczema, unspecified |
| | cause |
| 667 | Mother 0-12 weeks DHEA SULPHATE |
| 668 | Child diagnosed Intestinal malabsorption |
| 669 | Mother 0-12 weeks PROLACTIN |
| 670 | Child diagnosed Blepharitis |
| 671 | Mother 24-40 weeks LYMPHOCYTES %-DIF |
| 672 | Mother 0-12 weeks FERRITIN |
| 673 | Mother count Symbicort/duoresp |
| 674 | Mother Pre-pregnancy PROTEIN C ACTIVITY |
| 675 | Mother 0-12 weeks HCT/HGB Ratio |
| 676 | Mother Pre-pregnancy CHOLESTEROL/HDL |
| 677 | Child count Metronidazole |
| 678 | Mother 12-24 weeks NORMOBLAST.abs |
| 679 | Father median Cholesterol/hdl |
| 680 | Mother 24-40 weeks ALBUMIN |
| 681 | Child diagnosed Candidiasis of skin and nails |
| 682 | Mother last Diastolic Blood Pressure 12-24 weeks |
| 683 | Mother 0-12 weeks RDW-CV |
| 684 | Mother 12-24 weeks URIC ACID |
| 685 | Apidoral given at birth |
| 686 | Mother 12-24 weeks BILIRUBIN TOTAL |
| 687 | Child diagnosed Irritable infant |
| 688 | Child diagnosed Varicella without mention of complication |
| 689 | Mother 0-12 weeks BILIRUBIN TOTAL |
| 690 | Father diagnosed Other and unspecified hyperlipidemia |
| 691 | Child diagnosed Infective otitis externa |
| 692 | Child diagnosed Insect bite |
| 693 | Mother Pre-pregnancy ANTI CARDIOLIPIN IgM |
| 694 | Child diagnosed Stenosis and insufficiency of lacrimal passages |
| 695 | Mother 24-40 weeks APTT-sec |
| 696 | Mother 24-40 weeks VITAMIN D (25-OH) |
| 697 | Mother 24-40 weeks GLOBULIN |
| 698 | Mother Pre-pregnancy CA-125 |
| 699 | Child diagnosed Acute and unspecified inflammation of lacrimal |
| | passages |
| 700 | Mother count Cetirizine |
| 701 | Child diagnosed Anal fissure |
| 702 | Child diagnosed Impetigo |
| 703 | Child diagnosed Laceration/cut |
| 704 | Mother 12-24 weeks APTT-sec |
| 705 | Mother 12-24 weeks LDH |
| 706 | Child diagnosed Contact dermatitis and other eczema |
| 707 | Mother 24-40 weeks CK—CREAT.KINASE(CPK) |
| 708 | Child diagnosed Serous otitis media; glue |
| 709 | Mother 0-12 weeks BILIRUBIN-DIRECT |
| 710 | Mother 12-24 weeks GPT (ALT) |
| 711 | Child count Fluticasone |
| 712 | Mother Pre-pregnancy APTT-R |
| 713 | Mother 24-40 weeks FIBRINOGEN CALCU |
| 714 | Mother 12-24 weeks NORMOBLAST.% |
| 715 | Child diagnosed Injuries |
| 716 | Mother 0-12 weeks CHOLESTEROL- HDL |
| 717 | Mother count Desogestrel |
| 718 | Mother Pre-pregnancy EOSINOPHILS %-DIF |
| 719 | Child diagnosed Wheezing baby syndrome |
| 720 | Mother 24-40 weeks FOLIC ACID |
| 721 | Mother Pre-pregnancy IgA |
| 722 | Child diagnosed Croup |
| 723 | Mother Pre-pregnancy PROT-S ANTIGEN (FREE |
| 724 | Mother count Lansoprazole |
| 725 | Mother 12-24 weeks CHOLESTEROL-LDL calc |
| 726 | Child diagnosed Diseases and other conditions of the tongue |
| 727 | Mother 12-24 weeks ALPHA FETOPROTEIN TM |
| 728 | Mother 12-24 weeks GLUCOSE 50 g |
| 729 | Mother 0-12 weeks HbF |
| 730 | Locality type: Collective Moshav |
| 731 | Child diagnosed Abnormal loss of weight |
| 732 | Child diagnosed Other diseases of nasal cavity and sinuses |
| 733 | Mother BMI delta 0-12 weeks to 12-24 weeks |
| 734 | Mother 0-12 weeks BILIRUBIN INDIRECT |
| 735 | Mother Weight delta 12-24 weeks to 24-40 weeks |
| 736 | Child diagnosed Acute laryngitis |
| 737 | Locality type: Jewish Locality 20,000-49,999 residents |
| 738 | Mother count Cefuroxime |
| 739 | Mother 12-24 weeks CALCIUM |
| 740 | Father diagnosed Essential hypertension |
| 741 | Mother Pre-pregnancy MONOCYTES abs-DIF |
| 742 | Child diagnosed Umbilical hernia without mention of |
| | obstruction or gangrene |
| 743 | Child diagnosed Allergy/allergic react nos |
| 744 | Child diagnosed Congenital musculoskeletal deformities of |
| | sternocleidomastoid |
| 745 | Child diagnosed Other speech disturbance |
| 746 | Mother 12-24 weeks RDW-CV |
| 747 | Mother 0-12 weeks PCT |
| 748 | Mother Pre-pregnancy LYMPHOCYTES %-DIF |
| 749 | Mother 24-40 weeks NORMOBLAST.abs |
| 750 | Child diagnosed Enterobiasis |
| 751 | Mother Pre-pregnancy FIBRINOGEN |
| 752 | Mother count Cefalexin |
| 753 | Child count Ceftriaxone |
| 754 | Mother Pre-pregnancy CHLORIDE |
| 755 | Mother count Progesterone |
| 756 | Locality type: Jewish Other Rural Locality |
| 757 | Child diagnosed Other and unspecified chronic nonsuppurative |
| | otitis media |
| 758 | Mother 12-24 weeks GOT (AST) |
| 759 | Mother 12-24 weeks PDW |
| 760 | Locality type: Jewish Locality 2,000-4,999 residents |
| 761 | Father diagnosed Morbid obesity |
| 762 | Mother Pre-pregnancy BLOOD TYPE |
| 763 | Mother 0-12 weeks HbA2 |
| 764 | Mother Weight delta 0-12 weeks to 12-24 weeks |
| 765 | Mother 24-40 weeks NON-HDL_CHOLESTEROL |
| 766 | Mother 12-24 weeks HDW |
| 767 | Mother Pre-pregnancy GLOM.FILTR.RATE |
| 768 | Child diagnosed Otalgia |
| 769 | Child diagnosed Unspecified otitis media |
| 770 | Premature birth |
| 771 | Child diagnosed Unsp.adv.effect of drug, medicinal/biological |
| | substance n.e.s. |
| 772 | Mother Pre-pregnancy VITAMIN D (25-OH) |
| 773 | Mother 24-40 weeks CHOLESTEROL-LDL calc |
| 774 | Mother 12-24 weeks CHLORIDE |
| 775 | Born in Israel |
| 776 | Mother 12-24 weeks CHOLESTEROL- HDL |
| 777 | Mother Pre-pregnancy HbA2 |
| 778 | Mother 0-12 weeks CHLORIDE |
| 779 | Locality type: Communal Locality |
| 780 | Mother Pre-pregnancy LIC |
| 781 | Locality type: Jewish Locality 5,000-9,999 residents |
| 782 | Mother 24-40 weeks NORMOBLAST.% |
| 783 | Locality type: Jewish Locality 500,000 and more residents |
| 784 | Locality type: Kibbutz |
| 785 | Locality type: Moshav 2,000-4,999 residents |
| 786 | Mother 0-12 weeks NORMOBLAST.% |
| 787 | Mother Pre-pregnancy NORMOBLAST.% |
| 788 | Locality type: Non-Jewish Locality 20,000-49,999 residents |
| 789 | Child diagnosed Urticaria |
| 790 | Mother Pre-pregnancy LIC % |
| 791 | Mother 24-40 weeks LI |
| 792 | Mother Pre-pregnancy NEUTROPHILS abs-DIF |
| 793 | Mother Pre-pregnancy TOXOPLASMA IgG |
| 794 | Locality type: Non-Jewish Locality 50,000-99,999 residents |
| 795 | Mother 24-40 weeks CONTROL PTT |
| 796 | Mother 12-24 weeks NON-HDL_CHOLESTEROL |
| 797 | Mother Pre-pregnancy HbF |
| 798 | Child diagnosed Vomiting (excl.preg. w06) |
| 799 | Mother Pre-pregnancy NEUTROPHILS %-DIF |
| 800 | Father Height count |
| 801 | Mother Pre-pregnancy MONOCYTES %-DIF |
| 802 | Mother Pre-pregnancy LYMPHOCYTES abs-DIF |
| 803 | Mother 12-24 weeks PHOSPHORUS |
| 804 | Mother 12-24 weeks HbA |
| 805 | Mother Pre-pregnancy HEMOGLOBIN A |
| 806 | Mother 24-40 weeks GGT |
| 807 | Mother 12-24 weeks BILIRUBIN-DIRECT |
| 808 | Ethnicity: Africa |
| 809 | Mother 0-12 weeks HbA |
| 810 | Child diagnosed Viral pneumonia |
| 811 | Ethnicity: Mediterranean |
| 812 | Child diagnosed Viral exanthem, unspecified |
| 813 | Mother 24-40 weeks FIBRINOGEN |
| 814 | Ethnicity: Latin America |
| 815 | Child diagnosed Torticollis, unspecified |
| 816 | Child diagnosed Congenital dislocation of hip |
| 817 | Mother 0-12 weeks NORMOBLAST.abs |
| 818 | Mother count Carbamazepine |
| 819 | Mother count Norgestimate and ethinylestradiol |
| 820 | Mother count Norethisterone |
| 821 | Mother count Nitrofurantoin |
| 822 | Mother count Metronidazole |
| 823 | Mother count Methylphenidate |
| 824 | Mother count Medroxyprogesterone |
| 825 | Mother count Loratadine |
| 826 | Mother count Ipratropium bromide |
| 827 | Mother count Gestodene and ethinylestradiol |
| 828 | Mother count Follitropin beta |
| 829 | Mother count Fluoxetine |
| 830 | Mother count Fluconazole |
| 831 | Mother count Fexofenadine |
| 832 | Mother count Famotidine |
| 833 | Mother count Escitalopram |
| 834 | Mother 0-12 weeks PT-INR |
| 835 | Mother count Dydrogesterone |
| 836 | Mother count Drospirenone and ethinylestradiol |
| 837 | Mother count Doxycycline |
| 838 | Mother count Dexamethasone |
| 839 | Mother count Desogestrel and ethinylestradiol |
| 840 | Mother count Desloratadine |
| 841 | Mother count Colchicine |
| 842 | Mother count Clonazepam |
| 843 | Mother count Clomifene |
| 844 | Mother count Clarithromycin |
| 845 | Mother count Citalopram |
| 846 | Mother count Ciprofloxacin |
| 847 | Mother count Chorionic gonadotrophin |
| 848 | Mother count Paroxetine |
| 849 | Child diagnosed Hand, foot, and mouth disease |
| 850 | Mother count Prednisone |
| 851 | Mother 12-24 weeks TRANSFERRIN |
| 852 | Child diagnosed Chronic serous otitis media |
| 853 | Child diagnosed Cellulitis and abscess of unspecified sites |
| 854 | Child diagnosed Cellulitis and abscess of finger |
| 855 | Child diagnosed Candidiasis of unspecified site |
| 856 | Child diagnosed Candidiasis of mouth |
| 857 | Child diagnosed Blisters with epidermal loss, burn |
| | 2nd.deg.unspecified site |
| 858 | Child diagnosed Convulsions |
| 859 | Child diagnosed Delivery in a completely normal case |
| 860 | Child diagnosed Anemia other/unspecified |
| 861 | Child diagnosed Allergy, unspecified, not elsewhere classified |
| 862 | Child diagnosed Allergic rhinitis |
| 863 | Child diagnosed Agranulocytosis |
| 864 | Child diagnosed Dermatophytosis of the body |
| 865 | Child diagnosed Disorders relating to other preterm infants |
| 866 | Mother count Progyluton cd |
| 867 | Child diagnosed Enteritis due to specified virus |
| 868 | Child diagnosed Acute myringitis without mention of otitis media |
| 869 | Child diagnosed Acute laryngotracheitis |
| 870 | Child diagnosed Feeding difficulties and mismanagement |
| 871 | Child diagnosed Acquired deformities of other parts of limbs |
| 872 | Child diagnosed Accident/injury; nos |
| 873 | Child diagnosed Abnormal weight gain |
| 874 | Mother count Triptorelin |
| 875 | Mother count Simvastatin |
| 876 | Mother count Sertraline |
| 877 | Mother count Seretide cd |
| 878 | Mother count Salbutamol |
| 879 | Child diagnosed Gastrointestinal hemorrhage |
| 880 | Mother count Choriogonadotropin alfa |
| 881 | Child diagnosed Hemangioma of unspecified site |
| 882 | Child diagnosed Tongue tie |
| 883 | Mother count Budesonide |
| 884 | Child diagnosed Nonsuppurative otitis media, not specified as |
| | acute or chronic |
| 885 | Child diagnosed Open wound of face, without mention of |
| | complication |
| 886 | Mother 12-24 weeks GLOBULIN |
| 887 | Child diagnosed Other serum reaction, not elsewhere classified |
| 888 | Child diagnosed Other specified erythematous conditions |
| 889 | Mother 12-24 weeks BILIRUBIN INDIRECT |
| 890 | Child diagnosed Other specified viral exanthemata |
| 891 | Child diagnosed Other symptoms involving digestive system |
| 892 | Father count Rosuvastatin |
| 893 | Father count Ramipril-hydrochlorothiazide cd |
| 894 | Father count Ramipril |
| 895 | Father count Propranolol |
| 896 | Father count Nifedipine-cd |
| 897 | Father count Nifedipine |
| 898 | Father count Metformin and sitagliptin cd |
| 899 | Mother 0-12 weeks GLOM.FILTR.RATE |
| 900 | Father count Insulin glargine |
| 901 | Child diagnosed Posttraumatic wound infection not elsewhere |
| | classified |
| 902 | Father count Bisoprolol |
| 903 | Father count Atorvastatin |
| 904 | Father count Atenolol |
| 905 | Child diagnosed Premat/immature liveborn infant |
| 906 | Child diagnosed Seborrhea |
| 907 | Child diagnosed Seborrheic dermatitis, unspecified |
| 908 | Mother 12-24 weeks RUBELLA Ab IgG |
| 909 | Child diagnosed Sneezing/nasal congestion |
| 910 | Child diagnosed Stomatitis |
| 911 | Child diagnosed Strabismus and other disorders of binocular eye |
| | movements |
| 912 | Mother Pre-pregnancy NORMOBLAST.abs |
| 913 | Child diagnosed Nervousness |
| 914 | Child diagnosed Laxity of ligament |
| 915 | Mother 0-12 weeks ESR |
| 916 | Child diagnosed Hypermetropia |
| 917 | Mother count Bethamethasone |
| 918 | Mother count Anti-d (rh) immunoglobulin |
| 919 | Mother count Aciclovir |
| 920 | Child diagnosed Herpangina |
| 921 | Mother 12-24 weeks BLOOD TYPE |
| 922 | Mother 24-40 weeks BLOOD TYPE |
| 923 | Child count Ranitidine |
| 924 | Child count Phenoxymethylpenicillin |
| 925 | Child count Mebendazole |
| 926 | Child count Loratadine |
| 927 | Child diagnosed Hip symptoms/complaints |
| 928 | Child diagnosed Hydrocele |
| 929 | Child diagnosed Hydronephrosis |
| 930 | Child count Cefaclor |
| 931 | Mother 12-24 weeks HCT/HGB Ratio |
| 932 | Child diagnosed Infectious mononucleosis |
| 933 | Child count Aciclovir |
| 934 | Father diagnosed Unspecified essential hypertension |
| 935 | Father diagnosed Overweight (bmi <30) |
| 936 | Father diagnosed Other abnormal glucose |
| 937 | Father diagnosed Lipid metabolism disorder |
| 938 | Father diagnosed Impaired fasting glucose |
| 939 | Father diagnosed Disorders of lipoid metabolism |
| 940 | Father diagnosed Diabetes mellitus without mention of |
| | complication |
| 941 | Child diagnosed Inguinal hernia, without mention of obstruction or |
| | gangrene |
| 942 | Father diagnosed Adult-onset type diabetes mellitus whithout |
| | complication |
| 943 | Child diagnosed Insect bite, nonvenomous face, neck, scalp |
| | without infection |
| 944 | Child diagnosed Jaundice, unspecified, not of newborn |
| 945 | Mother count Lamotrigine |

Table 1.2 presents a list of 620 parameters from which parameters for feeing the machine learning procedure can be selected when the subject is when the subject is an unborn subject. The list is sorted according the significance of the respective feature for predicting the likelihood for childhood obesity, in descending order, so that from the standpoint of prediction accuracy it is more preferred to select a parameter that is listed higher in Table 1.2, than a parameter that is listed lower in Table 1.2. For example, when N parameters are used, it is preferred to select those parameters from lines 1 through M of Table 1.2, where N≤M≤620.

TABLE 1.2

| No. | Parameter |
|---|---|
| 1 | Siblings median BMI zscore mean |
| 2 | Siblings mean BMI zscore mean |
| 3 | Siblings max BMI zscore mean |
| 4 | Father BMI median |
| 5 | Father BMI max |
| 6 | Siblings at 5 years of age BMI zscore mean |
| 7 | Siblings min BMI zscore mean |
| 8 | Father BMI mean |
| 9 | Father BMI min |
| 10 | Mother Pre-Pregnancy BMI max |
| 11 | Mother Pre-Pregnancy BMI median |
| 12 | Mother 24-40 weeks MCV |
| 13 | Mother Pre-Pregnancy BMI mean |
| 14 | Mother 24-40 weeks MCH |
| 15 | Age of Father at birth |
| 16 | Siblings count BMI zscore std |
| 17 | Mother Pre-Pregnancy BMI min |
| 18 | Mother last BMI 24-40 weeks |
| 19 | Age of Mother at birth |
| 20 | Father Weight median |
| 21 | Mother Pre-Pregnancy Weight max |
| 22 | Mother last BMI 0-12 weeks |
| 23 | Father Height median |
| 24 | Mother Pre-Pregnancy Weight mean |
| 25 | Mother 12-24 weeks VITAMIN B12 |
| 26 | Mother 0-12 weeks MCH |
| 27 | Father std Cholesterol |
| 28 | Mother Pre-Pregnancy Weight median |
| 29 | Siblings min BMI zscore std |
| 30 | Mother Pre-Pregnancy Weight min |
| 31 | Mother Pre-pregnancy CMV IgG |
| 32 | Mother Pre-pregnancy PDW |
| 33 | Mother 24-40 weeks GLUCOSE 50 g |
| 34 | Mother Pre-pregnancy GGT |
| 35 | Father Height mean |
| 36 | Siblings mean BMI zscore std |
| 37 | Father max Triglycerides |
| 38 | Mother 12-24 weeks RBC |
| 39 | Mother 0-12 weeks WBC |
| 40 | Siblings std BMI zscore mean |
| 41 | Mother last Diastolic Blood Pressure 24-40 weeks |
| 42 | Mother 12-24 weeks HB |
| 43 | Mother 12-24 weeks LUC % |
| 44 | Mother 0-12 weeks VITAMIN B12 |
| 45 | Mother 0-12 weeks HCT |
| 46 | Mother Pre-pregnancy GLUCOSE 50 g |
| 47 | Father mean Cholesterol- hdl |
| 48 | Father mean Triglycerides |
| 49 | Father Height min |
| 50 | Siblings count BMI zscore mean |
| 51 | Mother 0-12 weeks LYMP.abs |
| 52 | Mother Pre-pregnancy GLUCOSE |
| 53 | Mother last BMI 12-24 weeks |
| 54 | Father std Glucose |
| 55 | Mother Pre-pregnancy CK—CREAT.KINASE(CPK) |
| 56 | Father std Cholesterol-ldl calc |
| 57 | Father min Cholesterol- hdl |
| 58 | Mother last BMI Pre-pregnancy |
| 59 | Mother Pre-pregnancy TSH |
| 60 | Mother last Weight Pre-pregnancy |
| 61 | Mother Pre-pregnancy MCHC |
| 62 | Mother Pre-pregnancy LYMP.abs |
| 63 | Siblings median BMI zscore std |
| 64 | Mother 12-24 weeks IRON |
| 65 | Mother count Roxithromycin |
| 66 | Mother last Weight 12-24 weeks |
| 67 | Mother 24-40 weeks MPV |
| 68 | Mother 12-24 weeks GLUCOSE |
| 69 | Mother Pre-pregnancy PT % |
| 70 | Mother 24-40 weeks VITAMIN B12 |
| 71 | Father max Glucose |
| 72 | Father Weight max |
| 73 | Mother 24-40 weeks EOS % |
| 74 | Mother 24-40 weeks GLUCOSE (GTT) 0′ |
| 75 | Mother Pre-pregnancy HCT |
| 76 | Mother Pre-pregnancy BILIRUBIN-DIRECT |
| 77 | Mother 0-12 weeks MPV |
| 78 | Siblings max BMI zscore std |
| 79 | Father mean Glucose |
| 80 | Mother 0-12 weeks NEUT.abs |
| 81 | Father Weight mean |
| 82 | Mother Pre-pregnancy T4- FREE |
| 83 | Mother 24-40 weeks RBC |
| 84 | Mother Pre-pregnancy LYM % |
| 85 | Mother 24-40 weeks ALK. PHOSPHATASE |
| 86 | Mother 0-12 weeks EOS.abs |
| 87 | Father min Triglycerides |
| 88 | Mother 0-12 weeks MONO.abs |
| 89 | Mother Pre-pregnancy MPV |
| 90 | Mother Pre-pregnancy NEUT % |
| 91 | Mother 24-40 weeks APTT-R |
| 92 | Siblings at 13 years of age BMI zscore mean |
| 93 | Mother Pre-pregnancy PHOSPHORUS |
| 94 | Father count Metformin |
| 95 | Mother Pre-pregnancy NEUT.abs |
| 96 | Mother 12-24 weeks MCHC |
| 97 | Mother 24-40 weeks HEMOGLOBIN A1C % |
| 98 | Mother Pre-pregnancy CHOLESTEROL-LDL calc |
| 99 | Mother last Systolic Blood Pressure 0-12 weeks |
| 100 | Father median Triglycerides |
| 101 | Mother 24-40 weeks MICRO % |
| 102 | Mother last Systolic Blood Pressure 12-24 weeks |
| 103 | Mother 24-40 weeks MONO.abs |
| 104 | Mother 12-24 weeks PLT |
| 105 | Mother Pre-pregnancy LDH |
| 106 | Mother 0-12 weeks HEPATITIS Bs Ab |
| 107 | Mother Pre-pregnancy PLT |
| 108 | Father min Glucose |
| 109 | Father max Non-hdl_cholesterol |
| 110 | Mother 12-24 weeks NEUT % |
| 111 | Mother 24-40 weeks HYPO % |
| 112 | Mother last Systolic Blood Pressure Pre-pregnancy |
| 113 | Father Height max |
| 114 | Mother last Systolic Blood Pressure 24-40 weeks |
| 115 | Father median Cholesterol- hdl |
| 116 | Mother 12-24 weeks T4- FREE |
| 117 | Mother Pre-pregnancy UREA |
| 118 | Mother Pre-pregnancy MAGNESIUM |
| 119 | Mother 0-12 weeks CHOLESTEROL/HDL |
| 120 | Mother 24-40 weeks LYM % |
| 121 | Mother 12-24 weeks MCV |
| 122 | Mother Pre-pregnancy MONO.abs |
| 123 | Mother Pre-pregnancy WBC |
| 124 | Mother 12-24 weeks MONO.abs |
| 125 | Mother 24-40 weeks HCT |
| 126 | Mother 0-12 weeks CMV IgG |
| 127 | Mother 24-40 weeks PLT |
| 128 | Mother Pre-pregnancy PROTEIN-TOTAL |
| 129 | Mother 12-24 weeks CMV IgG |
| 130 | Mother 24-40 weeks CMV IgG |
| 131 | Mother 0-12 weeks SODIUM |
| 132 | Mother 24-40 weeks NEUT % |
| 133 | Mother 24-40 weeks MCHC |
| 134 | Father Weight min |
| 135 | Mother count Amoxicillin |
| 136 | Father mean Cholesterol |
| 137 | Father median Glucose |
| 138 | Mother Pre-pregnancy CHOLESTEROL |
| 139 | Mother 0-12 weeks MONO % |
| 140 | Mother 24-40 weeks LYMP.abs |
| 141 | Mother 12-24 weeks NEUT.abs |
| 142 | Mother Pre-pregnancy HYPER % |
| 143 | Mother 12-24 weeks TSH |
| 144 | Mother count Cabergoline |
| 145 | Mother last Weight 0-12 weeks |
| 146 | Mother Pre-pregnancy PCT |
| 147 | Father Height std |
| 148 | Mother 0-12 weeks TRIGLYCERIDES |
| 149 | Mother 0-12 weeks GLUCOSE |
| 150 | Father std Cholesterol/hdl |
| 151 | Mother Pre-pregnancy HYPO % |
| 152 | Mother 24-40 weeks FERRITIN |
| 153 | Father BMI std |
| 154 | Mother Pre-pregnancy BASO % |
| 155 | Mother 24-40 weeks SODIUM |
| 156 | Mother Pre-pregnancy VITAMIN B12 |
| 157 | Mother 0-12 weeks ESTRADIOL (E-2) |
| 158 | Mother 0-12 weeks LYM % |
| 159 | Mother 12-24 weeks EOS % |
| 160 | Mother 24-40 weeks NEUT.abs |
| 161 | Mother 24-40 weeks NEUTROPHILS abs-DIF |
| 162 | Father diagnosed Diabetes mellitus |
| 163 | Mother Pre-pregnancy CREATININE |
| 164 | Father Weight std |
| 165 | Mother 24-40 weeks HB |
| 166 | Mother BMI delta 12-24 weeks to 24-40 weeks |
| 167 | Mother 0-12 weeks GGT |
| 168 | Mother 0-12 weeks LH |
| 169 | Mother 24-40 weeks RDW |
| 170 | Mother 12-24 weeks HbA2 |
| 171 | Mother 0-12 weeks MCV |
| 172 | Mother Pre-pregnancy MONO % |
| 173 | Mother Pre-pregnancy HB |
| 174 | Mother 24-40 weeks LUC % |
| 175 | Mother count Enoxaparin |
| 176 | Mother 24-40 weeks MONO % |
| 177 | Mother 0-12 weeks NEUT % |
| 178 | Mother 24-40 weeks WBC |
| 179 | Father mean Non-hdl_cholesterol |
| 180 | Mother 0-12 weeks EOS % |
| 181 | Mother 0-12 weeks RDW |
| 182 | Mother Pre-pregnancy RDW |
| 183 | Mother 12-24 weeks LYM % |
| 184 | Mother Pre-pregnancy SHBG |
| 185 | Mother Pre-pregnancy FOLIC ACID |
| 186 | Mother 0-12 weeks HYPO % |
| 187 | Mother Pre-pregnancy MICRO % |
| 188 | Mother 24-40 weeks BILIRUBIN TOTAL |
| 189 | Mother Pre-pregnancy SODIUM |
| 190 | Mother Pre-pregnancy RBC |
| 191 | Mother 24-40 weeks BASO % |
| 192 | Mother 24-40 weeks LYMPHOCYTES abs-DIF |
| 193 | Mother 0-12 weeks PROGESTERONE |
| 194 | Father BMI count |
| 195 | Mother Pre-pregnancy TRIGLYCERIDES |
| 196 | Father max Cholesterol |
| 197 | Mother 12-24 weeks LYMP.abs |
| 198 | Mother last Diastolic Blood Pressure 0-12 weeks |
| 199 | Mother Pre-pregnancy GLOBULIN |
| 200 | Mother 24-40 weeks CREATININE |
| 201 | Father max Cholesterol-ldl calc |
| 202 | Father max Cholesterol- hdl |
| 203 | Mother Pre-pregnancy ESR |
| 204 | Mother 12-24 weeks PT-SEC |
| 205 | Mother 24-40 weeks LUC abs |
| 206 | Mother 24-40 weeks MPXI |
| 207 | Mother Pre-Pregnancy BMI std |
| 208 | Mother 12-24 weeks FERRITIN |
| 209 | Mother 0-12 weeks MPXI |
| 210 | Mother 0-12 weeks TSH |
| 211 | Mother 24-40 weeks GOT (AST) |
| 212 | Mother 24-40 weeks HYPER % |
| 213 | Mother 24-40 weeks EOSINOPHILS abs-DIF |
| 214 | Mother 12-24 weeks WBC |
| 215 | Father mean Cholesterol-ldl calc |
| 216 | Father std Triglycerides |
| 217 | Mother Pre-pregnancy HDW |
| 218 | Mother 0-12 weeks UREA |
| 219 | Mother 12-24 weeks HCT |
| 220 | Mother Pre-pregnancy HEPATITIS Bs Ab |
| 221 | Mother 0-12 weeks LDH |
| 222 | Mother 12-24 weeks POTASSIUM |
| 223 | Mother Pre-Pregnancy Weight std |
| 224 | Mother 12-24 weeks MICRO % |
| 225 | Mother Pre-pregnancy BILIRUBIN TOTAL |
| 226 | Mother 0-12 weeks HB |
| 227 | Mother Pre-pregnancy C-REACTIVE PROTEIN |
| 228 | Mother Pre-pregnancy MCV |
| 229 | Mother Pre-pregnancy DHEA SULPHATE |
| 230 | Father min Cholesterol |
| 231 | Mother Pre-pregnancy EOS % |
| 232 | Father median Cholesterol |
| 233 | Mother 24-40 weeks BILIRUBIN-DIRECT |
| 234 | Mother 24-40 weeks STABS %-DIF |
| 235 | Siblings at 5 years of age BMI zscore std |
| 236 | Father std Cholesterol- hdl |
| 237 | Mother 12-24 weeks HYPO % |
| 238 | Mother 24-40 weeks STABS abs-DIF |
| 239 | Mother 0-12 weeks LUC % |
| 240 | Mother 12-24 weeks SODIUM |
| 241 | Mother 24-40 weeks GLUCOSE (GTT) 60′ |
| 242 | Mother 24-40 weeks CHOLESTEROL |
| 243 | No. of Siblings with BMI data |
| 244 | Mother 12-24 weeks CREATININE |
| 245 | Mother 24-40 weeks GLUCOSE (GTT) 180′ |
| 246 | Mother 12-24 weeks EOS.abs |
| 247 | Mother Pre-pregnancy COMPLEMENT C3 |
| 248 | Mother Pre-pregnancy EOS.abs |
| 249 | Mother 24-40 weeks T3- FREE |
| 250 | Mother Pre-pregnancy FERRITIN |
| 251 | Mother Pre-pregnancy AMYLASE |
| 252 | Father count Pravastatin |
| 253 | Mother 24-40 weeks MONOCYTES abs-DIF |
| 254 | Mother 24-40 weeks GPT (ALT) |
| 255 | Mother Pre-pregnancy URIC ACID |
| 256 | Father diagnosed Obesity, unspecified |
| 257 | Mother 24-40 weeks NEUTROPHILS %-DIF |
| 258 | Mother 0-12 weeks MCHC |
| 259 | Mother 12-24 weeks MONO % |
| 260 | Mother Pre-pregnancy FIBRINOGEN CALCU |
| 261 | Mother Pre-pregnancy MPXI |
| 262 | Mother 0-12 weeks URIC ACID |
| 263 | Mother Pre-pregnancy LH |
| 264 | Mother 24-40 weeks MACRO % |
| 265 | Mother Pre-pregnancy MCH |
| 266 | Mother 24-40 weeks BASO abs |
| 267 | Father count Cholesterol-ldl calc |
| 268 | Mother 0-12 weeks MICRO % |
| 269 | Mother Weight delta Pre-pregnancy to 0-12 weeks |
| 270 | Siblings std BMI zscore std |
| 271 | Mother 24-40 weeks LDH |
| 272 | Mother 0-12 weeks PLT |
| 273 | Siblings at 13 years of age BMI zscore std |
| 274 | Father count Glucose |
| 275 | Mother Pre-pregnancy BILIRUBIN INDIRECT |
| 276 | Mother 24-40 weeks URIC ACID |
| 277 | Mother BMI delta Pre-pregnancy to 0-12 weeks |
| 278 | Mother 12-24 weeks GGT |
| 279 | Mother 0-12 weeks GPT (ALT) |
| 280 | Mother 0-12 weeks PHOSPHORUS |
| 281 | Mother Pre-pregnancy LUC % |
| 282 | Mother 0-12 weeks HYPER % |
| 283 | Mother 0-12 weeks CREATININE |
| 284 | Mother 12-24 weeks MICRO %/HYPO % |
| 285 | Mother 0-12 weeks MACRO % |
| 286 | Mother 12-24 weeks RDW |
| 287 | Mother Pre-pregnancy POTASSIUM |
| 288 | Mother 0-12 weeks RBC |
| 289 | Mother Pre-pregnancy ALK. PHOSPHATASE |
| 290 | Mother Pre-pregnancy ALBUMIN |
| 291 | Mother 12-24 weeks TRIGLYCERIDES |
| 292 | Mother 0-12 weeks AMYLASE |
| 293 | Father min Cholesterol-ldl calc |
| 294 | Mother 0-12 weeks ALK. PHOSPHATASE |
| 295 | Mother Pre-pregnancy PT-SEC |
| 296 | Mother 0-12 weeks VITAMIN D (25-OH) |
| 297 | Mother 12-24 weeks MCH |
| 298 | Mother Pre-pregnancy CALCIUM |
| 299 | Father count Cholesterol- hdl |
| 300 | Father median Cholesterol-ldl calc |
| 301 | Mother Pre-pregnancy COMPLEMENT C4 |
| 302 | Mother count Ofloxacin |
| 303 | Mother last Weight 24-40 weeks |
| 304 | Mother 0-12 weeks CHOLESTEROL-LDL calc |
| 305 | Mother Pre-pregnancy MACRO % |
| 306 | Mother count Phenoxymethylpenicillin |
| 307 | Mother 0-12 weeks HDW |
| 308 | Mother 24-40 weeks TRIGLYCERIDES |
| 309 | Mother Pre-pregnancy TESTOSTERONE- TOTAL |
| 310 | Father std Non-hdl_cholesterol |
| 311 | Mother 0-12 weeks NON-HDL_CHOLESTEROL |
| 312 | Mother last Diastolic Blood Pressure Pre-pregnancy |
| 313 | Mother 0-12 weeks APTT-sec |
| 314 | Mother 24-40 weeks MICRO %/HYPO % |
| 315 | Mother 12-24 weeks MPXI |
| 316 | Mother 0-12 weeks BASO % |
| 317 | Father min Non-hdl_cholesterol |
| 318 | Mother Pre-pregnancy NON-HDL_CHOLESTEROL |
| 319 | Mother 0-12 weeks GLOBULIN |
| 320 | Mother 12-24 weeks MACRO % |
| 321 | Father count Simvastatin |
| 322 | Mother 12-24 weeks LUC abs |
| 323 | Mother 12-24 weeks PT-INR |
| 324 | Mother 0-12 weeks GOT (AST) |
| 325 | Father min Cholesterol/hdl |
| 326 | Mother 24-40 weeks GLUCOSE |
| 327 | Mother 24-40 weeks EOS.abs |
| 328 | Mother 12-24 weeks UREA |
| 329 | Mother 0-12 weeks PROTEIN-TOTAL |
| 330 | Mother Pre-pregnancy ALY |
| 331 | Mother Pre-pregnancy FREE ANDROGEN INDEX |
| 332 | Mother 0-12 weeks POTASSIUM |
| 333 | Mother 12-24 weeks AMYLASE |
| 334 | Mother 12-24 weeks CK—CREAT.KINASE(CPK) |
| 335 | Mother Pre-pregnancy GPT (ALT) |
| 336 | Mother 0-12 weeks CHOLESTEROL |
| 337 | Mother 12-24 weeks BASO % |
| 338 | Mother Pre-pregnancy CORTISOL-BLOOD |
| 339 | Mother 24-40 weeks RDW-CV |
| 340 | Mother Pre-pregnancy ESTRADIOL (E-2) |
| 341 | Mother 12-24 weeks MPV |
| 342 | Mother Pre-pregnancy PROLACTIN |
| 343 | Mother 24-40 weeks TSH |
| 344 | is Male |
| 345 | Mother 0-12 weeks CK—CREAT.KINASE(CPK) |
| 346 | Father median Non-hdl_cholesterol |
| 347 | Father mean Cholesterol/hdl |
| 348 | Mother 0-12 weeks FOLIC ACID |
| 349 | Mother 24-40 weeks IRON |
| 350 | Mother 0-12 weeks LUC abs |
| 351 | Mother Pre-pregnancy RUBELLA Ab IgG |
| 352 | Mother 0-12 weeks ALBUMIN |
| 353 | Mother 0-12 weeks IRON |
| 354 | Mother 0-12 weeks RUBELLA Ab IgG |
| 355 | Mother 24-40 weeks AMYLASE |
| 356 | Number of twin siblings |
| 357 | Mother Pre-pregnancy ANDROSTENEDIONE |
| 358 | Father count Enalapril |
| 359 | Mother count Mebendazole |
| 360 | Mother 24-40 weeks CHLORIDE |
| 361 | Mother 24-40 weeks HDW |
| 362 | Mother 24-40 weeks GLUCOSE (GTT) 120′ |
| 363 | Father count Cholesterol |
| 364 | Mother 12-24 weeks PCT |
| 365 | Mother 24-40 weeks UREA |
| 366 | Mother 0-12 weeks TOXOPLASMA IgG |
| 367 | Mother Pre-pregnancy MICRO %/HYPO % |
| 368 | Mother 24-40 weeks PROTEIN-TOTAL |
| 369 | Mother 12-24 weeks TOXOPLASMA IgG |
| 370 | Mother 0-12 weeks FSH |
| 371 | Father count Non-hdl_cholesterol |
| 372 | Mother 24-40 weeks CHOLESTEROL- HDL |
| 373 | Mother 24-40 weeks PT-SEC |
| 374 | Mother Pre-pregnancy ANTI CARDIOLIPIN IgG |
| 375 | Mother Pre-Pregnancy BMI count |
| 376 | Mother 24-40 weeks PDW |
| 377 | Mother 24-40 weeks MONOCYTES %-DIF |
| 378 | Mother 0-12 weeks MICRO %/HYPO % |
| 379 | Mother Pre-pregnancy TRANSFERRIN |
| 380 | Mother Pre-pregnancy GOT (AST) |
| 381 | Mother Pre-pregnancy PT-INR |
| 382 | Mother 24-40 weeks CALCIUM |
| 383 | Mother 0-12 weeks HEMOGLOBIN A |
| 384 | Mother Pre-pregnancy LUC abs |
| 385 | Father count Amlodipine |
| 386 | Mother 12-24 weeks ALK. PHOSPHATASE |
| 387 | Father count Triglycerides |
| 388 | Mother 0-12 weeks CALCIUM |
| 389 | Mother 12-24 weeks FOLIC ACID |
| 390 | Mother Pre-pregnancy FSH |
| 391 | Mother Pre-pregnancy CHOLESTEROL- HDL |
| 392 | Mother Pre-pregnancy PROGESTERONE |
| 393 | Mother 0-12 weeks T4- FREE |
| 394 | Mother 12-24 weeks BASO abs |
| 395 | Mother Pre-pregnancy ANTITHROMBIN-III |
| 396 | Mother 24-40 weeks TOXOPLASMA IgG |
| 397 | Mother 0-12 weeks PT-SEC |
| 398 | Mother Pre-pregnancy CONTROL PTT |
| 399 | Mother 24-40 weeks EOSINOPHILS %-DIF |
| 400 | Mother Pre-pregnancy 17-OH-PROGESTERONE |
| 401 | Father count Cholesterol/hdl |
| 402 | Mother Pre-pregnancy IRON |
| 403 | Mother Pre-pregnancy HEMOGLOBIN A1C % |
| 404 | Mother 12-24 weeks HYPER % |
| 405 | Mother 0-12 weeks BASO abs |
| 406 | Mother Pre-pregnancy APTT-sec |
| 407 | Mother count Fluticasone |
| 408 | Mother 24-40 weeks HCT/HGB Ratio |
| 409 | Father count Bezafibrate |
| 410 | Father diagnosed Obesity (bmi >30) |
| 411 | Mother count Omeprazole |
| 412 | Mother 24-40 weeks PT-INR |
| 413 | Mother Pre-pregnancy HCT/HGB Ratio |
| 414 | Mother Pre-Pregnancy Weight count |
| 415 | Mother count Estradiol |
| 416 | Mother 24-40 weeks PCT |
| 417 | Mother Pre-pregnancy T3-TOTAL |
| 418 | Mother count Follitropin alfa |
| 419 | Mother 24-40 weeks PT % |
| 420 | Mother Pre-pregnancy VLDL |
| 421 | Mother 24-40 weeks POTASSIUM |
| 422 | Mother 12-24 weeks HbF |
| 423 | Mother 24-40 weeks BILIRUBIN INDIRECT |
| 424 | Mother 24-40 weeks GLOM.FILTR.RATE |
| 425 | Mother 24-40 weeks PHOSPHORUS |
| 426 | Father max Cholesterol/hdl |
| 427 | Mother Pre-pregnancy ALY % |
| 428 | Mother 0-12 weeks PT % |
| 429 | Mother 12-24 weeks PT % |
| 430 | Mother 24-40 weeks TRANSFERRIN |
| 431 | Father Weight count |
| 432 | Mother Pre-pregnancy T3- FREE |
| 433 | Mother 12-24 weeks PROTEIN-TOTAL |
| 434 | Mother Pre-pregnancy BASO abs |
| 435 | Mother 0-12 weeks T3- FREE |
| 436 | Mother Pre-pregnancy RDW-CV |
| 437 | Mother count Levothyroxine sodium |
| 438 | Mother 12-24 weeks ALBUMIN |
| 439 | Mother 12-24 weeks CHOLESTEROL |
| 440 | Mother 24-40 weeks MAGNESIUM |
| 441 | Mother 0-12 weeks PDW |
| 442 | Mother 0-12 weeks TRANSFERRIN |
| 443 | Mother 24-40 weeks HbA2 |
| 444 | Mother 12-24 weeks T3- FREE |
| 445 | Mother count Aspirin |
| 446 | Mother 0-12 weeks BLOOD TYPE |
| 447 | Mother count Human menopausal gonadotrophin |
| 448 | Mother count Co-amoxiclav cd |
| 449 | Mother 24-40 weeks T4- FREE |
| 450 | Mother 0-12 weeks DHEA SULPHATE |
| 451 | Mother 0-12 weeks PROLACTIN |
| 452 | Mother 24-40 weeks LYMPHOCYTES %-DIF |
| 453 | Mother 0-12 weeks FERRITIN |
| 454 | Mother count Symbicort/duoresp |
| 455 | Mother Pre-pregnancy PROTEIN C ACTIVITY |
| 456 | Mother 0-12 weeks HCT/HGB Ratio |
| 457 | Mother Pre-pregnancy CHOLESTEROL/HDL |
| 458 | Mother 12-24 weeks NORMOBLAST.abs |
| 459 | Father median Cholesterol/hdl |
| 460 | Mother 24-40 weeks ALBUMIN |
| 461 | Mother last Diastolic Blood Pressure 12-24 weeks |
| 462 | Mother 0-12 weeks RDW-CV |
| 463 | Mother 12-24 weeks URIC ACID |
| 464 | Apidoral given at birth |
| 465 | Mother 12-24 weeks BILIRUBIN TOTAL |
| 466 | Mother 0-12 weeks BILIRUBIN TOTAL |
| 467 | Father diagnosed Other and unspecified hyperlipidemia |
| 468 | Mother Pre-pregnancy ANTI CARDIOLIPIN IgM |
| 469 | Mother 24-40 weeks APTT-sec |
| 470 | Mother 24-40 weeks VITAMIN D (25-OH) |
| 471 | Mother 24-40 weeks GLOBULIN |
| 472 | Mother Pre-pregnancy CA-125 |
| 473 | Mother count Cetirizine |
| 474 | Mother 12-24 weeks APTT-sec |
| 475 | Mother 12-24 weeks LDH |
| 476 | Mother 24-40 weeks CK—CREAT.KINASE(CPK) |
| 477 | Mother 0-12 weeks BILIRUBIN-DIRECT |
| 478 | Mother 12-24 weeks GPT (ALT) |
| 479 | Mother Pre-pregnancy APTT-R |
| 480 | Mother 24-40 weeks FIBRINOGEN CALCU |
| 481 | Mother 12-24 weeks NORMOBLAST.% |
| 482 | Mother 0-12 weeks CHOLESTEROL- HDL |
| 483 | Mother count Desogestrel |
| 484 | Mother Pre-pregnancy EOSINOPHILS %-DIF |
| 485 | Mother 24-40 weeks FOLIC ACID |
| 486 | Mother Pre-pregnancy IgA |
| 487 | Mother Pre-pregnancy PROT-S ANTIGEN (FREE |
| 488 | Mother count Lansoprazole |
| 489 | Mother 12-24 weeks CHOLESTEROL-LDL calc |
| 490 | Mother 12-24 weeks ALPHA FETOPROTEIN TM |
| 491 | Mother 12-24 weeks GLUCOSE 50 g |
| 492 | Mother 0-12 weeks HbF |
| 493 | Mother BMI delta 0-12 weeks to 12-24 weeks |
| 494 | Mother 0-12 weeks BILIRUBIN INDIRECT |
| 495 | Mother Weight delta 12-24 weeks to 24-40 weeks |
| 496 | Mother count Cefuroxime |
| 497 | Mother 12-24 weeks CALCIUM |
| 498 | Father diagnosed Essential hypertension |
| 499 | Mother Pre-pregnancy MONOCYTES abs-DIF |
| 500 | Mother 12-24 weeks RDW-CV |
| 501 | Mother 0-12 weeks PCT |
| 502 | Mother Pre-pregnancy LYMPHOCYTES %-DIF |
| 503 | Mother 24-40 weeks NORMOBLAST.abs |
| 504 | Mother Pre-pregnancy FIBRINOGEN |
| 505 | Mother count Cefalexin |
| 506 | Mother Pre-pregnancy CHLORIDE |
| 507 | Mother count Progesterone |
| 508 | Mother 12-24 weeks GOT (AST) |
| 509 | Mother 12-24 weeks PDW |
| 510 | Father diagnosed Morbid obesity |
| 511 | Mother Pre-pregnancy BLOOD TYPE |
| 512 | Mother 0-12 weeks HbA2 |
| 513 | Mother Weight delta 0-12 weeks to 12-24 weeks |
| 514 | Mother 24-40 weeks NON-HDL_CHOLESTEROL |
| 515 | Mother 12-24 weeks HDW |
| 516 | Mother Pre-pregnancy GLOM.FILTR.RATE |
| 517 | Premature birth |
| 518 | Mother Pre-pregnancy VITAMIN D (25-OH) |
| 519 | Mother 24-40 weeks CHOLESTEROL-LDL calc |
| 520 | Mother 12-24 weeks CHLORIDE |
| 521 | Born in Israel |
| 522 | Mother 12-24 weeks CHOLESTEROL- HDL |
| 523 | Mother Pre-pregnancy HbA2 |
| 524 | Mother 0-12 weeks CHLORIDE |
| 525 | Mother Pre-pregnancy LIC |
| 526 | Mother 24-40 weeks NORMOBLAST.% |
| 527 | Mother 0-12 weeks NORMOBLAST.% |
| 528 | Mother Pre-pregnancy NORMOBLAST.% |
| 529 | Mother Pre-pregnancy LIC % |
| 530 | Mother 24-40 weeks LI |
| 531 | Mother Pre-pregnancy NEUTROPHILS abs-DIF |
| 532 | Mother Pre-pregnancy TOXOPLASMA IgG |
| 533 | Mother 24-40 weeks CONTROL PTT |
| 534 | Mother 12-24 weeks NON-HDL_CHOLESTEROL |
| 535 | Mother Pre-pregnancy HbF |
| 536 | Mother Pre-pregnancy NEUTROPHILS %-DIF |
| 537 | Father Height count |
| 538 | Mother Pre-pregnancy MONOCYTES %-DIF |
| 539 | Mother Pre-pregnancy LYMPHOCYTES abs-DIF |
| 540 | Mother 12-24 weeks PHOSPHORUS |
| 541 | Mother 12-24 weeks HbA |
| 542 | Mother Pre-pregnancy HEMOGLOBIN A |
| 543 | Mother 24-40 weeks GGT |
| 544 | Mother 12-24 weeks BILIRUBIN-DIRECT |
| 545 | Mother 0-12 weeks HbA |
| 546 | Mother 24-40 weeks FIBRINOGEN |
| 547 | Mother 0-12 weeks NORMOBLAST.abs |
| 548 | Mother count Carbamazepine |
| 549 | Mother count Norgestimate and ethinylestradiol |
| 550 | Mother count Norethisterone |
| 551 | Mother count Nitrofurantoin |
| 552 | Mother count Metronidazole |
| 553 | Mother count Methylphenidate |
| 554 | Mother count Medroxyprogesterone |
| 555 | Mother count Loratadine |
| 556 | Mother count Ipratropium bromide |
| 557 | Mother count Gestodene and ethinylestradiol |
| 558 | Mother count Follitropin beta |
| 559 | Mother count Fluoxetine |
| 560 | Mother count Fluconazole |
| 561 | Mother count Fexofenadine |
| 562 | Mother count Famotidine |
| 563 | Mother count Escitalopram |
| 564 | Mother 0-12 weeks PT-INR |
| 565 | Mother count Dydrogesterone |
| 566 | Mother count Drospirenone and ethinylestradiol |
| 567 | Mother count Doxycycline |
| 568 | Mother count Dexamethasone |
| 569 | Mother count Desogestrel and ethinylestradiol |
| 570 | Mother count Desloratadine |
| 571 | Mother count Colchicine |
| 572 | Mother count Clonazepam |
| 573 | Mother count Clomifene |
| 574 | Mother count Clarithromycin |
| 575 | Mother count Citalopram |
| 576 | Mother count Ciprofloxacin |
| 577 | Mother count Chorionic gonadotrophin |
| 578 | Mother count Paroxetine |
| 579 | Mother count Prednisone |
| 580 | Mother 12-24 weeks TRANSFERRIN |
| 581 | Mother count Progyluton cd |
| 582 | Mother count Triptorelin |
| 583 | Mother count Simvastatin |
| 584 | Mother count Sertraline |
| 585 | Mother count Seretide cd |
| 586 | Mother count Salbutamol |
| 587 | Mother count Choriogonadotropin alfa |
| 588 | Mother count Budesonide |
| 589 | Mother 12-24 weeks GLOBULIN |
| 590 | Mother 12-24 weeks BILIRUBIN INDIRECT |
| 591 | Father count Rosuvastatin |
| 592 | Father count Ramipril-hydrochlorothiazide cd |
| 593 | Father count Ramipril |
| 594 | Father count Propranolol |
| 595 | Father count Nifedipine-cd |
| 596 | Father count Nifedipine |
| 597 | Father count Metformin and sitagliptin cd |
| 598 | Mother 0-12 weeks GLOM.FILTR.RATE |
| 599 | Father count Insulin glargine |
| 600 | Father count Bisoprolol |
| 601 | Father count Atorvastatin |
| 602 | Father count Atenolol |
| 603 | Mother 12-24 weeks RUBELLA Ab IgG |
| 604 | Mother Pre-pregnancy NORMOBLAST.abs |
| 605 | Mother 0-12 weeks ESR |
| 606 | Mother count Bethamethasone |
| 607 | Mother count Anti-d (rh) immunoglobulin |
| 608 | Mother count Aciclovir |
| 609 | Mother 12-24 weeks BLOOD TYPE |
| 610 | Mother 24-40 weeks BLOOD TYPE |
| 611 | Mother 12-24 weeks HCT/HGB Ratio |
| 612 | Father diagnosed Unspecified essential hypertension |
| 613 | Father diagnosed Overweight (bmi <30) |
| 614 | Father diagnosed Other abnormal glucose |
| 615 | Father diagnosed Lipid metabolism disorder |
| 616 | Father diagnosed Impaired fasting glucose |
| 617 | Father diagnosed Disorders of lipoid metabolism |
| 618 | Father diagnosed Diabetes mellitus without mention of |
| | complication |
| 619 | Father diagnosed Adult-onset type diabetes mellitus whithout |
| | complication |
| 620 | Mother count Lamotrigine |

Table 1.3 presents a list of 66 response parameters from which parameter to be included in questionnaire can be selected when the subject is an infant or toddler subject. The questionnaire can presented to a person on behalf of the subject, and can provide response parameters for feeing the machine learning procedure. The list is sorted according the significance of the respective feature for predicting the likelihood for childhood obesity, in descending order, so that from the standpoint of prediction accuracy it is more preferred to select a parameter that is listed higher in Table 1.3, than a parameter that is listed lower in Table 1.3. For example, when N parameters are used, it is preferred to select those parameters from lines 1 through M of Table 1.3, where N≤M≤66.

TABLE 1.3

| No. | Parameter |
|---|---|
| 1 | Last WFL zscore |
| 2 | Siblings mean BMI zscore mean |
| 3 | Father BMI mean |
| 4 | Weight Routine checkup - 18-22 months |
| 5 | Weight Routine checkup - 12-16 months |
| 6 | Weight Routine checkup - 4-6 months |
| 7 | Ethnicity: North Africa |
| 8 | Weight Routine checkup - 9-12 months |
| 9 | WFL Routine checkup - 18-22 months |
| 10 | WFL Routine checkup - 12-16 months |
| 11 | WFL Routine checkup - 1-2 months |
| 12 | Mother last BMI Pre-pregnancy |
| 13 | Date of Birth |
| 14 | WFL Routine checkup - 9-12 months |
| 15 | Age of Father at birth |
| 16 | Siblings mean BMI zscore std |
| 17 | Age of Mother at birth |
| 18 | Ethnicity: West Europe |
| 19 | Weight Routine checkup - 6-9 months |
| 20 | WFL Routine checkup - 4-6 months |
| 21 | Father Weight mean |
| 22 | WFL Routine checkup - 2-3 months |
| 23 | Mother last BMI 0-12 weeks |
| 24 | Mother last Weight Pre-pregnancy |
| 25 | Ethnicity: North America |
| 26 | Mother last BMI 24-40 weeks |
| 27 | No. of Siblings with BMI data |
| 28 | Weight Routine checkup - 2-3 months |
| 29 | Ethnicity: Unknown |
| 30 | WFL Routine checkup - 6-9 months |
| 31 | Height Routine checkup - 12-16 months |
| 32 | Ethnicity: Ethiopia |
| 33 | Height Routine checkup - 18-22 months |
| 34 | Ethnicity: East Europe |
| 35 | Week of year bom |
| 36 | Birth weight |
| 37 | Mother last BMI 12-24 weeks |
| 38 | Weight Routine checkup - 1-2 months |
| 39 | Height Routine checkup - 9-12 months |
| 40 | Age at last WFL |
| 41 | Age at Target measurement |
| 42 | Mother last Weight 12-24 weeks |
| 43 | Height Routine checkup - 2-3 months |
| 44 | Height Routine checkup - 6-9 months |
| 45 | Ethnicity: Iraq |
| 46 | Ethnicity: Muslim Arab |
| 47 | Height Routine checkup - 4-6 months |
| 48 | Mother BMI delta 12-24 weeks to 24-40 weeks |
| 49 | Height Routine checkup - 1-2 months |
| 50 | Mother last Weight 0-12 weeks |
| 51 | Ethnicity: Iran |
| 52 | Mother BMI delta Pre-pregnancy to 0-12 weeks |
| 53 | Mother last Weight 24-40 weeks |
| 54 | Mother Weight delta Pre-pregnancy to 0-12 weeks |
| 55 | Ethnicity: Asian |
| 56 | Ethnicity: Yemen |
| 57 | is Male |
| 58 | Mother Weight delta 0-12 weeks to 12-24 weeks |
| 59 | Ethnicity: USSR |
| 60 | Ethnicity: Mediterranean |
| 61 | Mother Weight delta 12-24 weeks to 24-40 weeks |
| 62 | Mother BMI delta 0-12 weeks to 12-24 weeks |
| 63 | Ethnicity: Latin America |
| 64 | Born in Israel |
| 65 | Premature birth |
| 66 | Ethnicity: Africa |

Table 1.4 presents a list of 21 response parameters from which parameter to be included in questionnaire can be selected when the subject is an unborn subject. The questionnaire can presented to a person on behalf of the subject, and can provide response parameters for feeing the machine learning procedure. The list is sorted according the significance of the respective feature for predicting the likelihood for childhood obesity, in descending order, so that from the standpoint of prediction accuracy it is more preferred to select a parameter that is listed higher in Table 1.4, than a parameter that is listed lower in Table 1.4. For example, when N parameters are used, it is preferred to select those parameters from lines 1 through M of Table 1.4, where N≤M≤21.

TABLE 1.4

| No. | Parameter |
|---|---|
| 1 | Siblings mean BMI zscore mean |
| 2 | Father BMI mean |
| 3 | Mother last BMI Pre-pregnancy |
| 4 | Age of Father at birth |
| 5 | Siblings mean BMI zscore std |
| 6 | Age of Mother at birth |
| 7 | Father Weight mean |
| 8 | Mother last BMI 0-12 weeks |
| 9 | Mother last Weight Pre-pregnancy |
| 10 | Mother last BMI 24-40 weeks |
| 11 | No. of Siblings with BMI data |
| 12 | Mother last BMI 12-24 weeks |
| 13 | Mother last Weight 12-24 weeks |
| 14 | Mother BMI delta 12-24 weeks to 24-40 weeks |
| 15 | Mother last Weight 0-12 weeks |
| 16 | Mother BMI delta Pre-pregnancy to 0-12 weeks |
| 17 | Mother last Weight 24-40 weeks |
| 18 | Mother Weight delta Pre-pregnancy to 0-12 weeks |
| 19 | Mother Weight delta 0-12 weeks to 12-24 weeks |
| 20 | Mother Weight delta 12-24 weeks to 24-40 weeks |
| 21 | Mother BMI delta 0-12 weeks to 12-24 weeks |

### Example 2

This Example describes analysis of data collected over a decade from Israel's largest healthcare provider, to assess risk factors for pediatric obesity and to develop a model for assessing children's obesity risk in order to inform and target interventions. The inventors analyzed nationwide electronic health records of children from 2006 to 2018 for whom sequential anthropometric data were available. Obesity was defined as body mass index (BMI)≥95th percentile for age and gender. Data of children and their families included anthropometric measurements, drug prescriptions, medical diagnoses, demographic data and laboratory tests.

Analysis of BMI trajectories among 382,132 adolescents revealed that among obese adolescents, the largest annual increase in BMI percentile occurs at 2-5 years of age. Therefore, the inventors devised a computational model based on data of 136,196 children from birth up to 2 years of age for predicting obesity at 5-6 years of age and from birth and up to 2 years of age. Most (51%) obese children in our cohort had a normal weight at infancy. As will be shown below, the model predicted obesity with an area under the receiver operating characteristic curve (auROC) and 95% CI of 0.803 [0.796−0.812]. Discrimination results on different subpopulations demonstrated its robustness across a clinically heterogeneous pediatric population. The most influential features included anthropometric measurements of the child and the family. Other impactful features included ethnicity and maternal pregnancy glucose measurements. A model based solely on features that are available pre-birth had similar performance to a model based on the child's last available weight and length measurements.

### Methods

### Study Design and Population

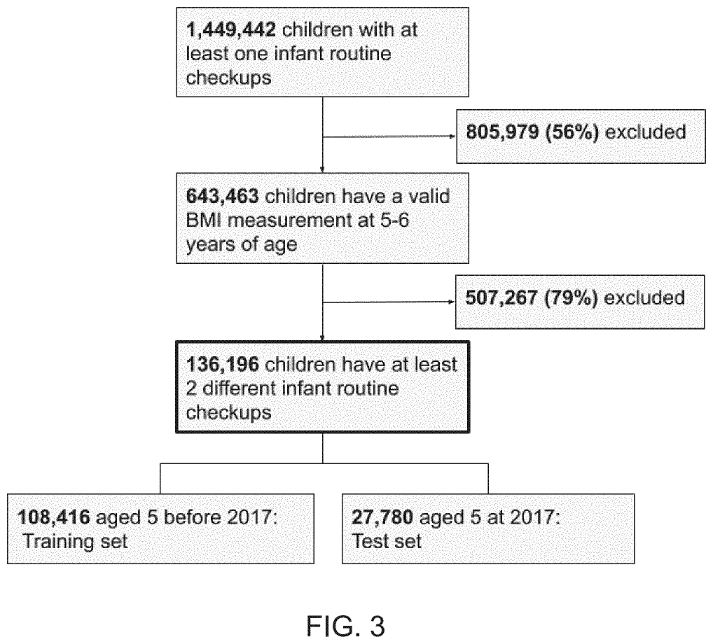

Extracted features included maternal, paternal and siblings' data. FIG. 3 illustrates the dataset used in the present Example. The dataset contained 1,449,442 children who have at least one measurement in a routine medical infant checkup which is scheduled for all Israeli infants at ages 1, 2, 4, 6, 9, 12, and 18 months. Of them, 643,463 children have an additional measurement between 5 and 6 years of age, which was defined as the outcome for the machine learning procedure. 136,196 children who have at least 2 different routine checkup measurements in addition to the 5-6 years old outcome measurement were included in the cohort. 90,270 children included in the cohort have maternal data, 92,152 have paternal data and 70,735 have data of at least one sibling.

### Features

All EHR data available were binned into time periods and statistical measures (e.g., median, max, slope) were taken as features for each period. Pharmaceutical prescriptions and clinical diagnoses were categorized by ATC codes (Anon n.d.) and ICD9 diagnosis codes, respectively, and counts in different time periods were taken as features. Weight, height, Weight-for-Length (WFL) and BMI data were converted to reference z-scores provided by the Center for Disease Control and Prevention (CDC) (Barlow and Expert Committee 2007). Valid measurements were defined as being in the range of 5 CDC standard deviation scores for weight and height. Features from maternal pregnancy were binned in alignment with the routine pregnancy tests schedule in Israel. Specific features of interest such as antibiotic prescriptions, ethnicity, and socioeconomic status surrogates were devised manually based on domain knowledge. Altogether, 943 features were devised for each child.

The characteristics of the Study Cohort and features used are summarized in Table 2.1, below.

TABLE 2.1

| | | |
|---|---|---|
| Train set | Temporal test set | |
| (n = 108,416) | (n = 27,780) | |
| aged 5 before 2017 | aged 5 at 2017 | All |

Children (n = 136,196)

| | | | | |
|---|---|---|---|---|
| Obesity status at 5-6 years | Underweight | 13,635 | 3,304 | 16,939 |
| of age | Normal weight | 75,648 | 19,867 | 95,515 |
| | Overweight | 19,133 | 4,609 | 23,742 |
| | Obese | 8,120 | 1,941 | 10,061 |
| Sex | Female | 52,733 | 13,458 | 66,191 |
| | Male | 55,683 | 14,322 | 70,005 |

Children with maternal data (n = 90,270)

| | | | | |
|---|---|---|---|---|
| Maternal age at childbirth | mean (std) | 30.1 (5.2) | 30.5 (5.2) | 30.1 (5.2) |
| [years] | | | | |
| Pre-pregnancy BMI | mean (std) | 23.6 (4.7) | 23.3 (4.4) | 23.5 (4.6) |
| [m/kg²] | | | | |

Children with paternal data (n = 92,152)

| | | | | |
|---|---|---|---|---|
| Paternal age [years] | mean (std) | 33.1 (5.9) | 33.3 (5.7) | 33.2 (5.9) |
| Paternal BMI [m/kg²] | mean (std) | 25.9 (4.4) | 25.6 (4.2) | 25.9 (4.3) |

Children with Siblings data (n = 70,735)

| | | | | |
|---|---|---|---|---|
| Number of children with | count | 55070 | 15665 | 70735 |
| siblings data | | | | |
| Number of siblings per | mean (std) | 1.1 (1.3) | 1.3 (1.4) | 1.2 (1.3) |
| child | | | | |
| Sibling BMI CDC z-score | mean (std) | 0.0 (1.1) | −0.1 (1.1) | 0.0 (1.1) |

### Outcome

The outcome for the models was the obesity status of children at 5 to 6 years of age. Obesity status was defined in accordance with health care professionals in Israel, using the CDC BMI reference percentiles. Cutoffs for normal weight, overweight, and obesity were determined using the CDC's standard thresholds of the 85th percentile for overweight and 95th percentile for obesity. Using other percentiles curves such as, but not limited to, the World Health Organization (WHO) WFL, and WHO BMI provided similar estimates of obesity risk as the CDC percentiles at 5 years of age.

### Statistical Analysis

Childhood Obesity Prediction Model

In this Example, Gradient Boosting trees were trained for providing the prediction. Trees allow nonlinear and multiple feature interactions to be captured, which may be important in obtaining an accurate prediction model. The parameters of the model were tuned using cross-validation on the training set. As stringent tests, both temporal and geographical validations were used, thus testing the performance of the model for distribution shifts over time and geographic location. The temporal validation set contained the most recent year in which the data were available. The geographical validation set contained all the clinics in the most populated and multiethnic city in Israel, Jerusalem. Unless stated otherwise, the reported results are on the temporal validation sets. Full results on both validation sets are available in Table 2.2, below.

As a baseline model for comparison the last WFL percentile routine checkup measurement available before 2 years of age was used, as current guidelines recommend that clinicians assess a child's current nutritional and obesity status by calculating WFL percentile or BMI percentile in children 0 to 2 years of age, or older than 2 years of age, respectively (Daniels et al. 2015). The WFL percentile thus emulates the information a caregiver has today to assess the current obesity status and future obesity risk of children younger than 2 years of age (Taveras et al. 2009). This variable also contains information of sex and age, as it standardizes by them. This variable itself is a predictor of the outcome, achieving an auROC of 0.749 and auPR of 0.223, and acts as a baseline to compare and improve upon.

Risk Factors Analysis from the Prediction Model

Risk factors were investigated by analyzing which features attribute to the model's prediction. To this end, the recently introduced SHAP (SHapley Additive exPlanation) method (Lundberg and Lee 2017; Lundberg et al. 2018) was used. The SHAP interprets the output of a machine learning model. A feature's Shapley value represents the average change in the model's output by conditioning on that feature when introducing features one at a time over all feature orderings. Shapley values were calculated individually for every child's feature. A property of Shapley values is that they are additive, meaning that the Shapley values of a child's features add up to the predicted log-odds of obesity for that child. In this Example, this value was transformed for each feature and each child to obtain a relative risk score.

Feature attributions were thus analyzed at the individual level, by examining plots of the Shapley value as a function of the feature value for all individuals. This method allowed capturing non-linear and continuous relations between a feature's impact on the prediction and the feature's value. A vertical spread in such a plot implies interaction with other features in the model, which would not have been attainable using a linear model. Building a model with many correlated features (e.g., a child's weight measurement at adjacent time points) is bound to suffer from severe collinearity of the features, and consequently the feature attributions will be spread across these related features. To tackle this, the additive property of Shapley values was used. Adding up the Shapely values of related features provided an analysis on this group of features. This provided better estimates of relevant risk scores. Another use of the additive property allows adding features according to groups and analyzing the model globally by taking the mean over absolute Shapely values of all children in each group of features. This gives insight on the impact of a feature group.

### Results

### Acceleration of BMI in Early Childhood

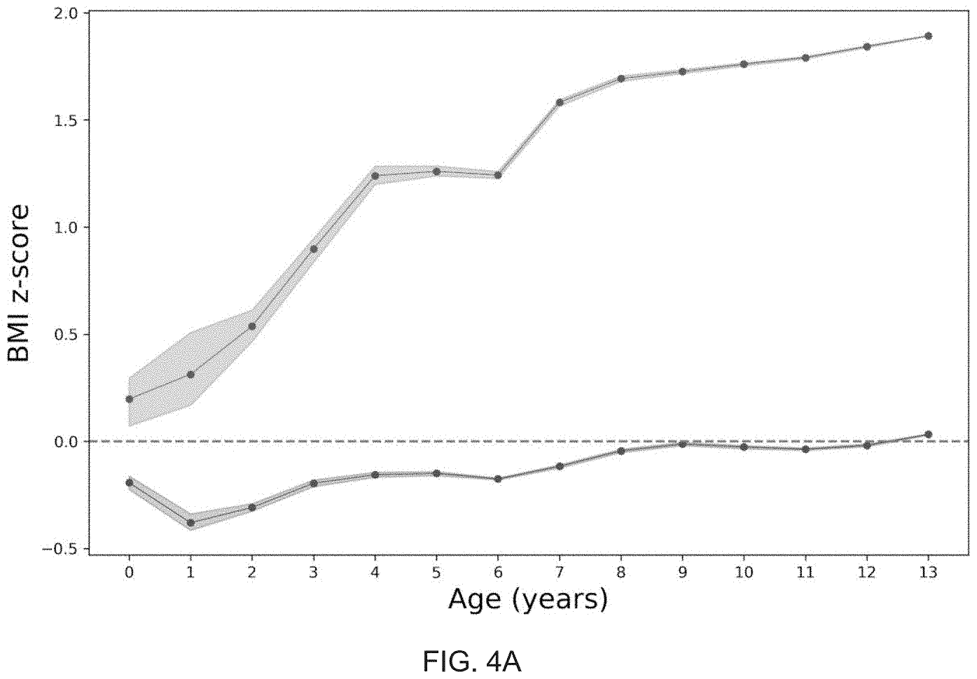

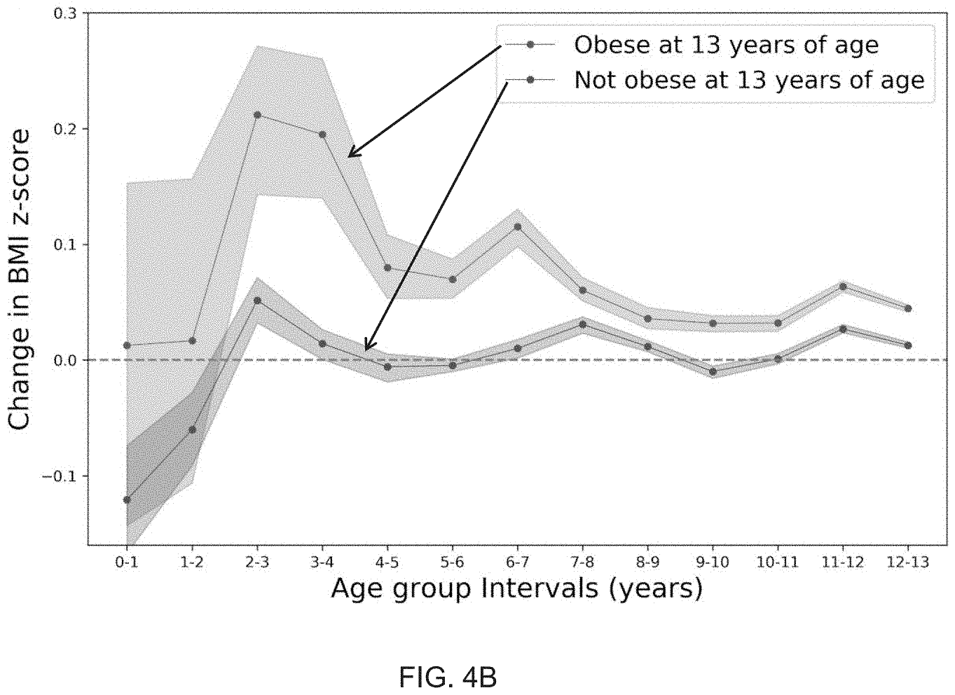

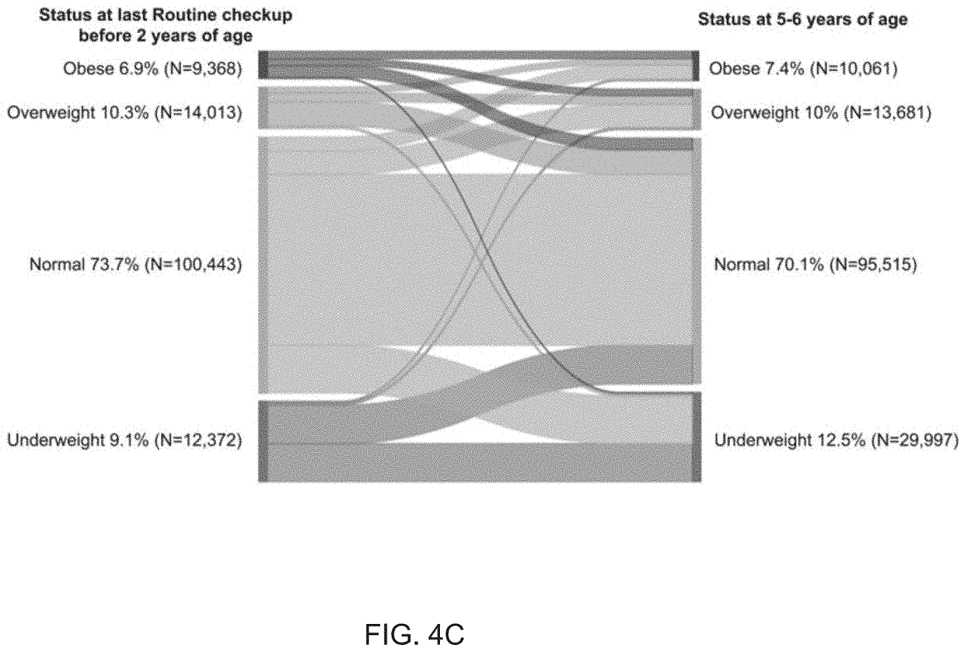

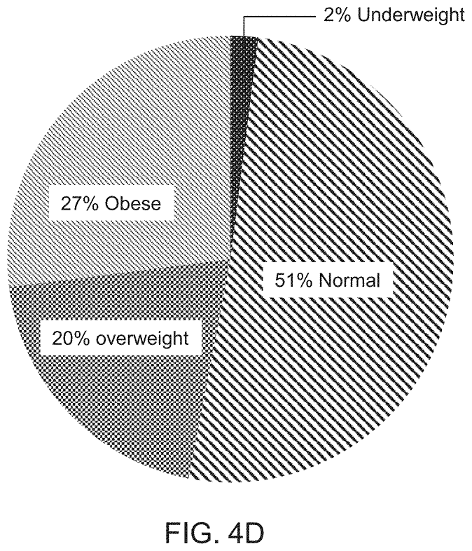

BMI trajectories were first analyzed in early childhood in relation to obesity status at 13-14 years of age. A total of 382,132 children with 1,401,803 measurements were included in the analysis (FIGS. 4A and 4B). The mean change in BMI z-score of children who were not obese at 13 years of age remained close to 0 from 1 year of age, with an annual change of less than 0.1 z-scores. However, for obese children at 13 years of age, the BMI z-score incremented throughout infancy and early childhood with the largest annual increase in BMI percentile observed at 2-5 years of age. A model has therefore been developed in accordance with some embodiments of the present invention to identify children at high risk for obesity within the subsequent 3-4 years at 2 years of age, prior to this critical time period.

The transition of obesity status over the first 6 years of life for the 136,196 children that were included in our cohort was analyzed. Obesity status was defined for each child at two time-points: the last available routine checkup before 2 years of age and at 5-6 years of age (FIG. 4C). This analysis revealed that most obese children at 5-6 years of age had normal weight at infancy (51%) (FIG. 4D).

### Prediction of Childhood Obesity at 5-6 Years of Age

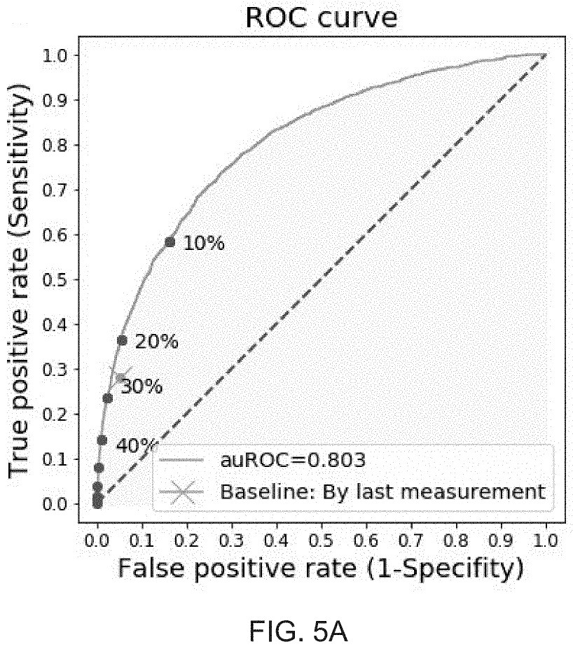

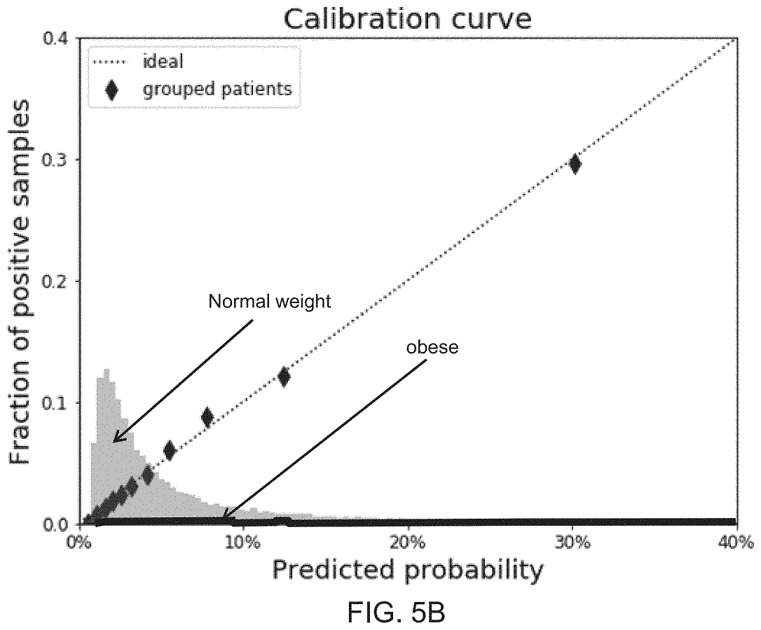

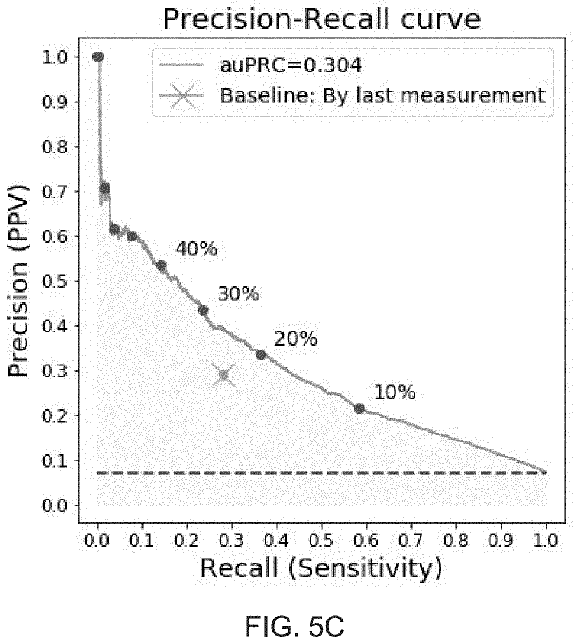

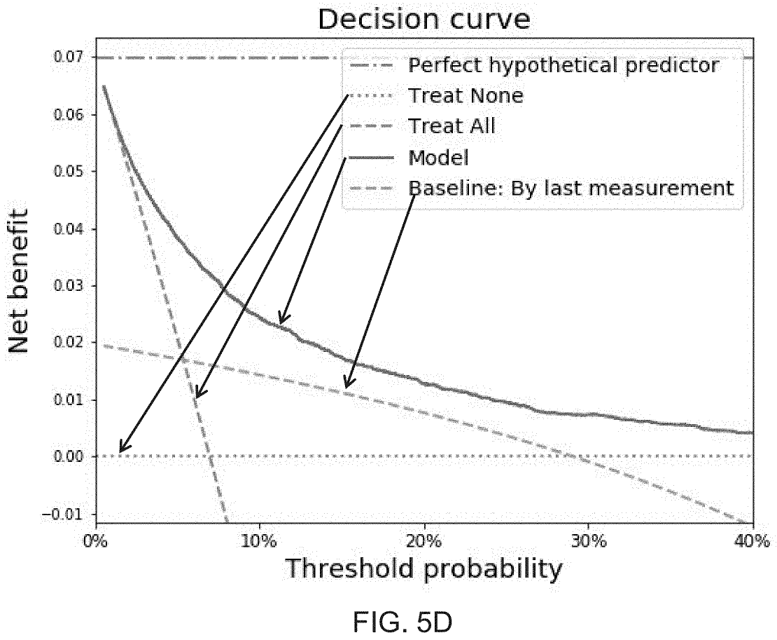

In accordance with some embodiments of the present invention, a model was constructed for predicting the likelihood for children at 0-2 years of age to develop childhood obesity at 5 to 6 years of age. The discrimination performance of the model was evaluated using the area under the receiver operating (auROC) and precision-recall (auPR) curves (FIGS. 5A and 5C). As shown, the technique of the present embodiments outperforms the baseline model based on the child's last WFL percentile. Both temporal and geographical validation results are summarized in Table 2.2, below.

The model of the present embodiments outputs calibrated continuous risk probabilities. Applying a clinical decision thereafter (for example, a nutritional intervention) can vary between individuals and depend on the costs and benefits of the action, both clinically and economically. Decision curves (Vickers and Elkin 2006) offer a graphical tool to analyze clinical utility of adopting a new risk prediction model. The curves contain information that can guide clinicians to make decisions based on the risk thresholds, and based on the tradeoffs (costs and benefits) of their decision to treat. The costs and benefits can be translated into a function of the optimal threshold probability. In this Example, clinical utility was analyzed by constructing decision curves (FIG. 5D). As shown, the model of the present embodiments dominates over other strategies in net benefit over all threshold probabilities, with significant margins in the lower threshold probability regime. A summary of the effect of applying different decision thresholds on the model performance is presented in Table 2.2, below.

The discrimination results (auPR) of the model of the present embodiments were further analyzed on different subpopulations of children (FIGS. 6A-C). The effect of gender on the performance of the model was evaluated. Similar results for boys and girls were found. Children who had at least one diagnosis of a complex chronic condition were evaluated using a previously defined classification system (Feudtner et al. 2014). The discrimination of the model was similar in this group, demonstrating the robustness of the model of the present embodiments across a clinically heterogeneous pediatric population. Discrimination performance was also evaluated by obesity status as defined by the last available child percentile prior to 2 years of age. The model of the present embodiments had the highest auPR in children who were obese at infancy, followed by overweight and normal weight at infancy. The model of the present embodiments outperformed the baseline model in predicting future obesity in all infants, regardless of obesity status at baseline (FIG. 6B). An increase in the number of documented anthropometric measurements during routine checkups improved the discrimination performance of the model.

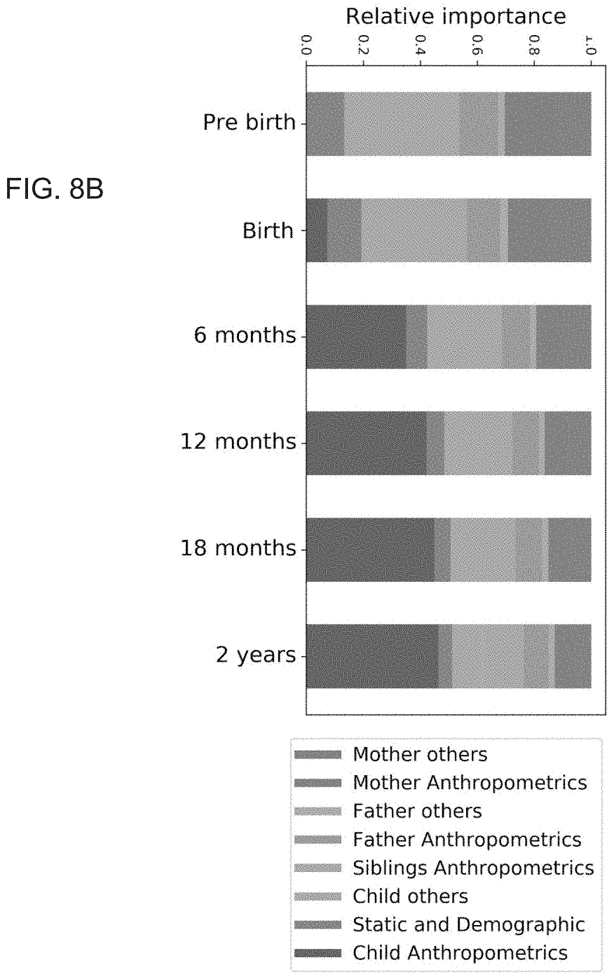

As earlier detection of childhood obesity may be more beneficial and allow earlier interventions, the ability to construct a prediction model for childhood obesity at the age 5-6 years of age was analyzed in the following time points: pre-birth, birth, 6 months, 1 year and 1.5 years of age. The effect of the child's age at prediction and the model discrimination performance is presented in FIG. 8A. As shown, the model performance improved when the prediction is done at an older age, which is closer to the target age of the predictor. Note that a prediction model constructed pre-birth has an auROC of 0.708 and auPR of 0.176, very similar to the performance of the baseline model based on the child's own weight and length measurements at 1 years of age which has an auROC of 0.709 and auPR of 0.166. The model of the present embodiments thus outperformed the baseline model in the entire age range.

### Features Attribution

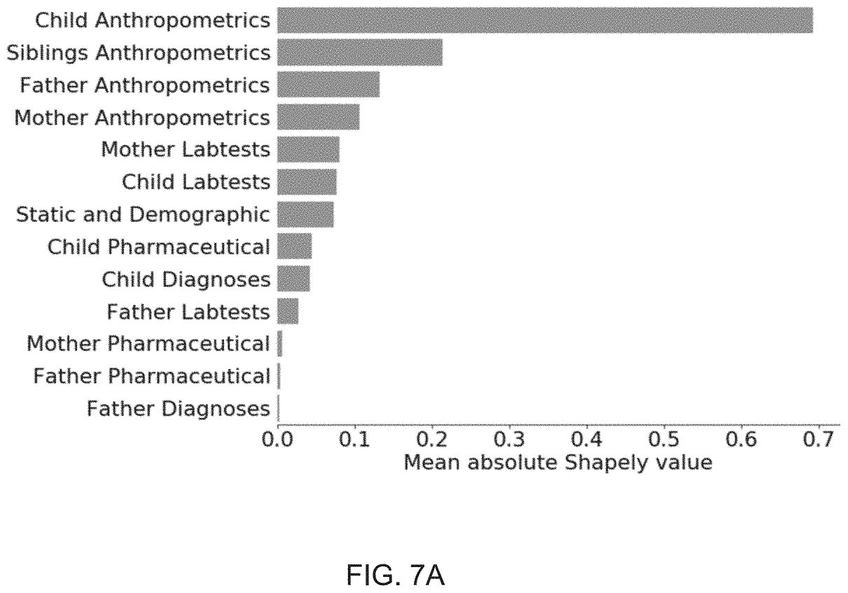

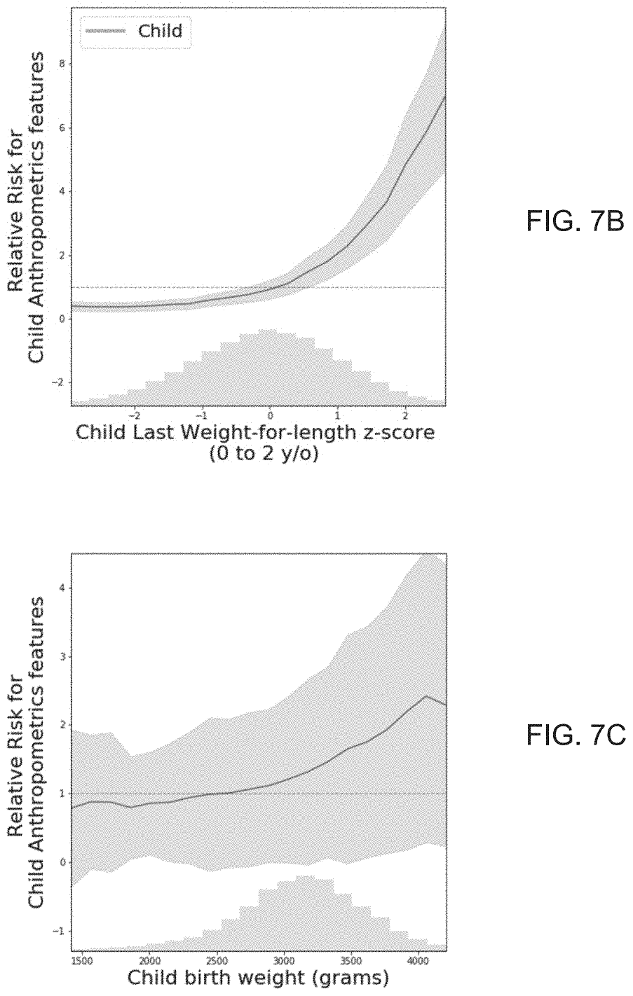

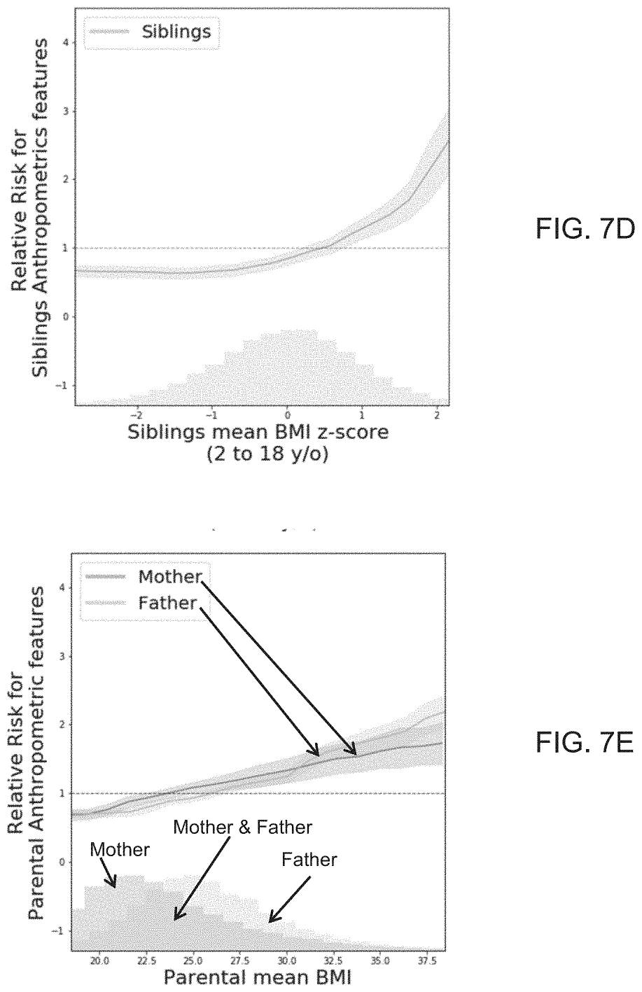

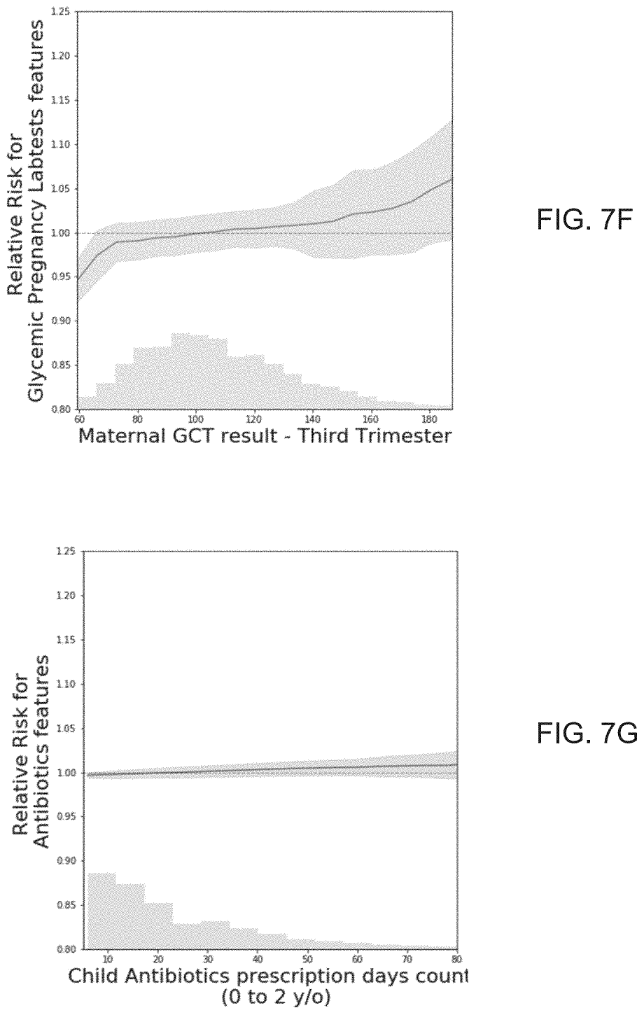

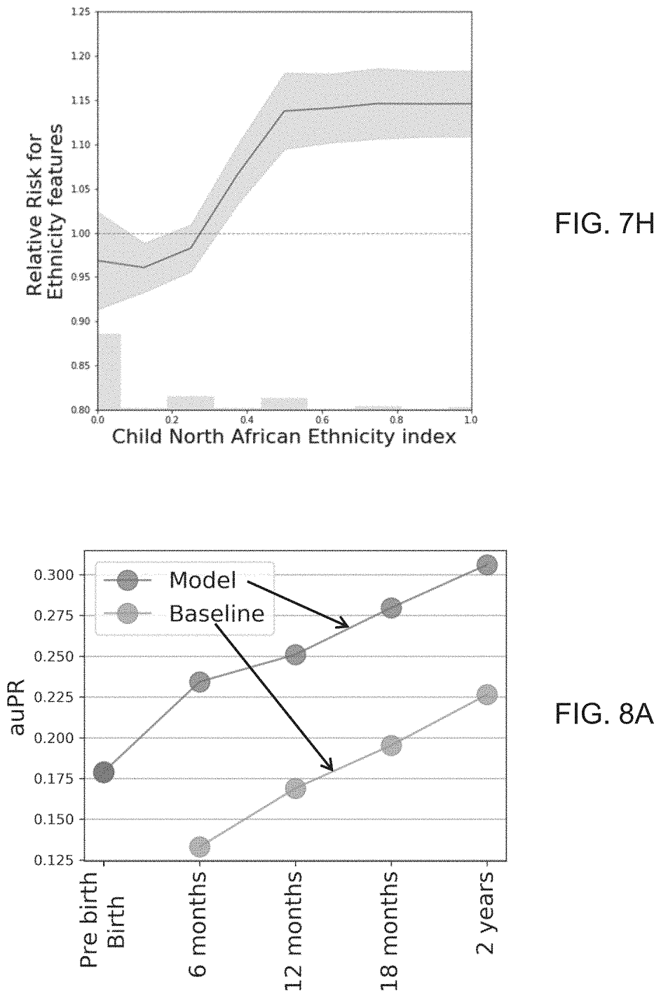

An analysis of feature attributions was performed using Shapley values. The results of the analysis are shown in FIGS. 7A-H. FIG. 7A presents a global analysis of the model's features attributions. The mean of absolute summation of Shapley values for different groups of features is presented for the entire cohort. Feature importance dependence plots of the Shapley value were also examined as a function of the feature value for all individuals. Most of the influential features were previous anthropometric measurements of the child, with the last measured WFL percentile being the most impactful feature (FIG. 7C). Anthropometric features of parents and siblings and North African Jewish descendancy also had a significant impact on the prediction (FIGS. 7A, 7D, 7E and 7H). Interestingly, maternal blood glucose on 50 g glucose tolerance tests (GTT) were also influential for the prediction of obesity at 5-6 years of age (FIG. 7F). Relative risk for obesity has increased monotonically across all the maternal glucose spectrum and increased above 1 in values above 100 mg/dL.

Analysis of the relative importance of different groups of features at different ages of applying the predictor revealed that the most influential features at birth are anthropometric measurements of the siblings, mother and father. Following these, the influence of the child's own anthropometrics measurements becomes more substantial and is roughly equal to the contribution of all other features in 1 years of age. Laboratory tests, drugs prescriptions and diagnoses have smaller relative influence, which decreases as the data on the child's anthropometrics accumulates (FIG. 8B).

Using information on pharmaceutical prescriptions, the effect of in utero and early life antibiotic exposure was also analyzed. 83,627 children (80%) had at least one antibiotic prescription in the first 2 years of life. The analysis revealed that antibiotic exposure in utero and in the first two years of life and age of first exposure to antibiotic had no effect on obesity risk at 5-6 years of age (FIG. 7G).

### Prediction Model Based on a Smaller Number of Parameters

Based on the observation that infant routine checkups, family anthropometric measurements, and ethnicity contribute most to the predictive power of the model, a simple prediction model was established based on a set of self-assessed questions that parents can easily fill out at different time points up to 2 years of age in order to assess their child's risk of obesity. This model achieved an auROC of 0.798 and auPR of 0.296, compared to 0.749 and 0.223, respectively, for the baseline model.

### Discussion

This Example demonstrates a diagnostic prediction model for pediatric obesity at 5-6 years of age based on a comprehensive nationwide EHR encompassing over 10 years of children and familial data. Overweight 5-year-olds are four times more likely to become obese later in life compared to normal-weight children, and weight in this age is considered to be a good indicator of the child's future metabolic health. The target age of prediction model presented in this Example is also supported by a recently published observation on children BMI trajectories (Geserick et al. 2018), which was also replicated in our cohort, showing 2 to 6 years of age as the maximal BMI acceleration time period. The model is therefore designed to identify children at risk prior to this critical time window, in which mature eating patterns become more developed as children reduce breast milk or formula consumption. In addition, the analysis of the transition in obesity status in the first 6 years of life revealed that most obese children had normal weight at infancy, underscoring the importance of building a tool that allows clinicians to identify high risk infants that are considered to have a normal weight at infancy but will develop obesity, as they will constitute the majority of obese children in the future.

The model presented in this Example achieved an auROC of 0.803 and auPR of 0.304. Further Analysis of prediction performance on subpopulations of the cohort demonstrated robustness in discrimination performance across the entire pediatric population, including children with complex chronic diseases. Unlike previous studies (Hammond et al. 2019), the results presented in this Example were similar for boys and girls. Additional models were further devised for predicting obesity prior to two years of age. High impact of family anthropometric measurements in determining future obesity risk of the child was demonstrated. This Example showed that a prediction model constructed pre-birth, which is mainly based on family anthropometric measurements has very similar performance of predicting at 1 years of age based on the child's last available weight and length measurements. A simple self-assessed questionnaire for childhood obesity prediction pre-birth achieved an auROC of 0.798 and auPR of 0.296.

The technique presented in this Example has several advantages over previous studies. The technique presented in this Example include full data on both the child, from pregnancy to 5-6 years of age, and his family, and is the first to be validated both temporally and geographically at different clinics on a national level, thus representing a wide target population. The technique presented in this Example is the first to assess clinical utility by constructing decision curves. To date, there are no clinical guidelines defining the risk threshold for obesity prediction. The definition of this threshold may be influenced by many factors, including the characteristics of the proposed intervention, the availability of resources for intervention and the prevalence of obesity in the target population, and will impact the sensitivity and specificity of the prediction model. The decision curve analysis presented in this Example may thus help in determining risk thresholds and the clinical usefulness of the model for different interventions.

The mechanisms involved in the development of obesity in children are complex and include genetic, environmental, and developmental factors. The large cohort of Israeli children represents a diverse and multi-ethnic population with genetic heterogeneity. Not surprisingly, many of the variables found to be important in the model were directly related to the child's previous anthropometric measurements. Familial anthropometric measurements, including paternal, maternal and sibling's BMI were also important, in line with previous studies showing associations between these variables and childhood obesity. Among familial data, sibling's BMI had the highest impact on the prediction model, most likely due to both genetic and environmental influences.

There is evidence that uterine environment may cause a permanent influence on fetus future health, and may lead to enhanced susceptibility to diseases later in life. This concept is defined as ‘gestational programming’ of the fetus, and is thought to be mediated by Epigenetic mechanisms (Desai et al. 2015; Desai and Hales 1997). The data on maternal pregnancy, including lab tests, diagnoses and medications was used to analyze associations of these features to obesity status of the offspring at 5-6 years of age. One of the most prominent features in pregnancy was maternal blood glucose values (FIG. 7F). An increase in maternal blood glucose levels during pregnancy, adjusted for other features incorporated in the model (such as maternal BMI), was associated with a higher risk for childhood obesity. This association, which was apparent even in glucose values which are considered in the normal range, demonstrates that exposure to higher glucose levels in utero throughout the entire maternal glucose spectrum is significantly associated with childhood glucose and insulin resistance of the offspring and is independently associated with childhood adiposity. Ethnicity as a risk factor has previously been studied in the UK and USA populations, in which a higher prevalence of obesity was found among children of African descent (Brophy et al. 2009). The analysis presented in This Example concentrated on the Israeli population, and revealed North African Jewish descendancy as a strong contributor for predicting obesity.

The role of the gut microbiota in obesity has been vastly studied in recent years (Castaner et al. 2018). Microbiome composition undergoes many changes during the first years of life (Stewart et al. 2018). Antibiotics, which are frequently prescribed in the pediatric population (Chai et al. 2012), can significantly alter the microbiome composition (Robinson and Young 2010). Therefore, several recent studies assessed the relationship between antibiotic usage in early life and childhood obesity. These resulted in conflicting findings (Shao et al. 2017). The large sample size and the data on antibiotic prescriptions in pregnancy and infancy used in this Example allowed to explore this association. The analysis presented in this Example revealed that while the vast majority (80%) of the cohort received antibiotics at least once by the age of 2 years of age, antibiotic exposure in utero and in the first two years of life, and age of first exposure to antibiotic, had no observed impact on the obesity risk at 5-6 years of age.

The data used in This Example is from a retrospective observational EHR. These may suffer from potential biases and are affected by a variety of healthcare processes. Sampling bias was minimized by choosing children based on the schedule of routine measurements of weight and height, which includes both measurements at 0-2 years of age and a measurement at 5-6 years of age.

It is noted that while the prediction model presented in this Example is based on data of Israeli children, the validation process, which included both a temporal and a geographical validation, the well-known universal risk factors for childhood obesity that were found in the analysis of the model, and the striking similarity of the analysis on BMI trajectories to an independent, recently published German cohort (Geserick et al. 2018), indicates that the results may be generalized to other populations as well.

TABLE 2.2
Prediction Results

| | Temporal test set | | Geographical test set | |
|---|---|---|---|---|
| Model | auPR | auROC | auPR | auROC |
| Baseline | 0.223 | 0.749 | 0.177 | 0.736 |
| | (0.209-0.235) | (0.739-0.758) | (0.162-0.201) | (0.712-0.755) |
| Full | 0.304 | 0.803 | 0.251 | 0.789 |
| Model | (0.286-0.321) | (0.796-0.812) | (0.230-0.280) | (0.771-0.805) |

Abbreviations: auPR/auROC—Area under the PR/ROC curve, PR—Precision-Recall, ROC—Receiver-Operator-Characteristic

TABLE 2.3
Effects of varying decision threshold on model performance

| | Predicted probability threshold | | | | | | |
|---|---|---|---|---|---|---|---|
| | 2% | 5% | 10% | 20% | 30% | 40% | Baseline |
| Sensitivity | 0.962 | 0.794 | 0.585 | 0.364 | 0.236 | 0.142 | 0.281 |
| Specificity | 0.257 | 0.651 | 0.840 | 0.946 | 0.977 | 0.991 | 0.949 |
| PPV | 0.089 | 0.146 | 0.215 | 0.334 | 0.435 | 0.533 | 0.291 |
| NPV | 0.989 | 0.977 | 0.964 | 0.952 | 0.945 | 0.939 | 0.946 |
| Net Benefit | 0.053 | 0.038 | 0.024 | 0.013 | 0.007 | 0.004 | |

Abbreviations: NPV—Negative predictive value, PPV—positive predictive value

TABLE 2.4
Prediction of obesity at 5-6 years of age prior to 2 years of age

| Age of applying | | Temporal test set | | Geographical test set | |
|---|---|---|---|---|---|
| prediction | | auPR | auROC | auPR | auROC |
| Pre-birth | Full | 0.176 | 0.708 | 0.134 | 0.680 |
| | Model | (0.168-0.188) | (0.689-0.723) | (0.125-0.153) | (0.660-0.704) |
| Birth | Full | 0.177 | 0.711 | 0.134 | 0.684 |
| | Model | (0.169-0.189) | (0.701-0.726) | (0.124-0.153) | (0.666-0.708) |
| 6 months | Baseline | 0.133 | 0.671 | 0.099 | 0.641 |
| | | (0.126-0.144) | (0.666-0.681) | (0.085-0.117) | (0.620-0.669) |
| | Full | 0.230 | 0.759 | 0.174 | 0.728 |
| | Model | (0.216-0.249) | (0.751-0.769) | (0.153-0.200) | (0.713-0.747) |
| 12 months | Baseline | 0.166 | 0.709 | 0.130 | 0.684 |
| | | (0.159-0.178) | (0.700-0.716) | (0.117-0.147) | (0.667-0.703) |
| | Full | 0.249 | 0.777 | 0.204 | 0.755 |
| | Model | (0.233-0.267) | (0.769-0.787) | (0.187-0.229) | (0.739-0.775) |
| 18 months | Baseline | 0.190 | 0.732 | 0.162 | 0.716 |
| | | (0.179-0.201) | (0.726-0.742) | (0.147-0.184) | (0.693-0.740) |
| | Full | 0.278 | 0.791 | 0.230 | 0.775 |
| | Model | (0.262-0.297) | (0.783-0.800) | (0.215-0.255) | (0.759-0.792) |
| 2 years | Baseline | 0.223 | 0.749 | 0.177 | 0.736 |
| | | (0.209-0.235) | (0.739-0.758) | (0.162-0.201) | (0.712-0.755) |
| | Full | 0.304 | 0.803 | 0.251 | 0.789 |
| | Model | (0.286-0.321) | (0.796-0.812) | (0.230-0.280) | (0.771-0.805) |

Abbreviations: auPR/auROC—Area under the PR/ROC curve, PR—Precision-Recall, ROC—Receiver-Operator-Characteristic

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

[1] NCD Risk Factor Collaboration (NCD-RisC) (2017). Worldwide trends in body-mass index, underweight, overweight, and obesity from 1975 to 2016: a pooled analysis of 2416 population-based measurement studies in 128.9 million children, adolescents, and adults. Lancet 390, 2627-2642.
[2] Ebbeling, C. B., Pawlak, D. B., and Ludwig, D. S. (2002). Childhood obesity: public-health crisis, common sense cure. Lancet 360, 473-482.
[3] Karnik, S., and Kanekar, A. (2012). Childhood obesity: a global public health crisis. Int. J. Prev. Med. 3, 1-7.
[4] Young-Hyman, D., Schlundt, D. G., Herman, L., De Luca, F., and Counts, D. (2001). Evaluation of the insulin resistance syndrome in 5- to 10-year-old overweight/obese African-American children. Diabetes Care 24, 1359-1364.
[5] Molnár, D. (2004). The prevalence of the metabolic syndrome and type 2 diabetes mellitus in children and adolescents. Int. J. Obes. Relat. Metab. Disord. 28 Suppl 3, S70-4.
[6] Sawyer, M. G., Harchak, T., Wake, M., and Lynch, J. (2011). Four-year prospective study of BMI and mental health problems in young children. Pediatrics 128, 677-684.
[7] Jeffery, R. W., Drewnowski, A., Epstein, L. H., Stunkard, A. J., Wilson, G. T., Wing, R. R., and Hill, D. R. (2000). Long-term maintenance of weight loss: current status. Health Psychol. 19, 5-16.
[8] Reinehr, T., Kleber, M., Lass, N., and Toschke, A. M. (2010). Body mass index patterns over 5 y in obese children motivated to participate in a 1-y lifestyle intervention: age as a predictor of long-term success. Am. J. Clin. Nutr. 91, 1165-1171.
[9] He, M., and Evans, A. (2007). Are parents aware that their children are overweight or obese? Do they care? Can. Fam. Physician 53, 1493-1499.
[10] Patel, A. I., Madsen, K. A., Maselli, J. H., Cabana, M. D., Stafford, R. S., and Hersh, A. L. (2010). Underdiagnosis of pediatric obesity during outpatient preventive care visits. Acad. Pediatr. 10, 405-409.
[11] Riley, M. R., Bass, N. M., Rosenthal, P., and Merriman, R. B. (2005). Underdiagnosis of pediatric obesity and underscreening for fatty liver disease and metabolic syndrome by pediatricians and pediatric subspecialists. J. Pediatr. 147, 839-842.
[12] Redsell, S. A., Atkinson, P. J., Nathan, D., Siriwardena, A. N., Swift, J. A., and Glazebrook, C. (2011). Preventing childhood obesity during infancy in UK primary care: a mixed-methods study of HCPs' knowledge, beliefs and practice. BMC Fam. Pract. 12, 54.
[13] Barlow, S. E., and Expert Committee (2007). Expert committee recommendations regarding the prevention, assessment, and treatment of child and adolescent overweight and obesity: summary report. Pediatrics 120 Suppl 4, S164-92.
[14] Cunningham, S. A., Kramer, M. R., and Narayan, K. M. V. (2014). Incidence of childhood obesity in the United States. N. Engl. J. Med. 370, 403-411.
[15] Gardner, D. S. L., Hosking, J., Metcalf, B. S., Jeffery, A. N., Voss, L. D., and Wilkin, T. J. (2009). Contribution of early weight gain to childhood overweight and metabolic health: a longitudinal study (EarlyBird 36). Pediatrics 123, e67-73.
[16] Geserick, M., Vogel, M., Gausche, R., Lipek, T., Spielau, U., Keller, E., Pfäffle, R., Kiess, W., and Körner, A. (2018). Acceleration of BMI in early childhood and risk of sustained obesity. N. Engl. J. Med. 379, 1303-1312.
[17] Data—Clalit Research Institute Available at: http://clalitresearch(dot)org/about-us/our-data/ [Accessed Oct. 7, 2018].
[18] Daniels, S. R., Hassink, S. G., and COMMITTEE ON NUTRITION (2015). The role of the pediatrician in primary prevention of obesity. Pediatrics 136, e275-92.
[19] WHOCC—Structure and principles Available at: https://www(dot)whocc(dot)no/atc/structure_and_principles/ [Accessed Oct. 12, 2018].
[20] Fedorov, V., Mannino, F., and Zhang, R. (2009). Consequences of dichotomization. Pharm. Stat. 8, 50-61.
[21] Ke, G., Meng, Q., Finley, T., Wang, T., Chen, W., Ma, W., Ye, Q., and Liu, T.-Y. (2017). LightGBM: A Highly Efficient Gradient Boosting Decision Tree. undefined.
[22] Saito, T., and Rehmsmeier, M. (2015). The precision-recall plot is more informative than the ROC plot when evaluating binary classifiers on imbalanced datasets. PLoS ONE 10, e0118432.
[23] Steyerberg, E. W., Vickers, A. J., Cook, N. R., Gerds, T., Gonen, M., Obuchowski, N., Pencina, M. J., and Kattan, M. W. (2010). Assessing the performance of prediction models: a framework for traditional and novel measures. Epidemiology 21, 128-138.
[24] Gerds, T. A., Cai, T., and Schumacher, M. (2008). The performance of risk prediction models. Biom. J. 50, 457-479.
[25] Niculescu-Mizil, A., and Caruana, R. (2005). Predicting good probabilities with supervised learning. In Proceedings of the 22nd international conference on Machine learning—ICML '05 (New York, N.Y., USA: ACM Press), pp. 625-632.
[26] Aggarwal, C. C. ed. (2015). Data Classification: Algorithms and Applications illustrated. (CRC Press).
[27] Moons, K. G. M., Altman, D. G., Reitsma, J. B., Ioannidis, J. P. A., Macaskill, P., Steyerberg, E. W., Vickers, A. J., Ransohoff, D. F., and Collins, G. S. (2015). Transparent Reporting of a multivariable prediction model for Individual Prognosis or Diagnosis (TRIPOD): explanation and elaboration. Ann. Intern. Med. 162, W1-73.
[28] Vickers, A. J., and Elkin, E. B. (2006). Decision curve analysis: a novel method for evaluating prediction models. Med. Decis. Making 26, 565-574.
[29] Vickers, A. J., Van Calster, B., and Steyerberg, E. W. (2016). Net benefit approaches to the evaluation of prediction models, molecular markers, and diagnostic tests. BMJ 352, i6.
[30] Lundberg, S. M., and Lee, S.-I. (2017). A Unified Approach to Interpreting Model Predictions.
[31] Lundberg, S. M., Erion, G. G., and Lee, S.-I. (2018). Consistent Individualized Feature Attribution for Tree Ensembles. arXiv.

What is claimed is:

1. A method of predicting likelihood for childhood obesity, comprising:
obtaining a plurality of parameters, wherein at least a few of said parameters characterize an infant or toddler subject;
accessing a computer readable medium storing a machine learning procedure trained for predicting likelihoods for childhood obesity;
feeding said procedure with said plurality of parameters; and
receiving from said procedure an output indicative of a likelihood that said infant or toddler subject is expected to develop childhood obesity, wherein said output is related non-linearly to said parameters.

2. The method according to claim 1, wherein said plurality of parameters comprises at least one parameter extracted from an electronic health record associated with said infant or toddler subject.

3. The method according to claim 1, comprising presenting to a user, by a user interface, a questionnaire and a set of questionnaire controls, receiving a set of response parameters entered by said user using said questionnaire controls, wherein said plurality of parameters comprises said response parameters.

4. The method according to claim 1, wherein said plurality of parameters comprises at least one parameter extracted from a body liquid test applied to said infant or toddler subject.

5. The method according to claim 1, wherein said plurality of parameters comprises at least one parameter characterizing a parent or a sibling of said infant or toddler subject.

6. The method according to claim 5, wherein said at least one parameter characterizing said parent comprise a parameter extracted from a body liquid test applied to said parent or sibling.

7. The method according to claim 1, wherein said plurality of parameters comprises at least one parameter extracted from a diagnosis previously recorded for said subject.

8. The method according to claim 1, wherein said plurality of parameters comprises at least one parameter indicative of a pharmaceutical prescribed for said infant or toddler subject.

9. The method according to claim 1, wherein said infant or toddler subject is less than two years of age.

10. The method according to claim 1, wherein said infant or toddler subject is not obese.

11. The method of claim 10, wherein said infant or toddler subject has a normal weight.

12. The method according to claim 1, wherein said plurality of parameters comprises a weight-for-length score of said infant or toddler subject.

13. The method according to claim 1, wherein said plurality of parameters comprise a weight of said infant or toddler subject at age of from about 4 to about 6 months, a weight of said infant or toddler subject at age of from about 12 to about 16 months, and a weight of said infant or toddler subject at age of from about 18 to about 22 months.

14. The method according to claim 1, wherein said plurality of parameters comprises a parameter pertaining to a body-mass-index of a sibling of said infant or toddler subject.

15. The method according to claim 1, wherein said plurality of parameters comprises a parameter pertaining to a body-mass-index of a father of said infant or toddler subject.

16. The method according to claim 1, wherein said plurality of parameters comprises a result of a hemoglobin concentration test applied to said infant or toddler subject.

17. The method according to claim 1, wherein said wherein said plurality of parameters comprises a result of a mean platelet volume test applied to said infant or toddler subject.

18. The method according to claim 1, wherein said plurality of parameters comprises at least 10 of the parameters listed in Table 1.1.

19. A method of predicting likelihood for childhood obesity, comprising:
obtaining a plurality of parameters characterizing at least one of a parent and a sibling of an unborn subject;
accessing a computer readable medium storing a machine learning procedure trained for predicting likelihoods for childhood obesity;
feeding said procedure with said plurality of parameters; and
receiving from said procedure an output indicative of a likelihood that said unborn subject is expected to develop childhood obesity after birth, wherein said output is related non-linearly to said parameters.

20. The method according to claim 19, wherein said plurality of parameters comprises at least one parameter extracted from an electronic health record associated with said at least one of said parent and said sibling.

21. The method according to claim 19, comprising presenting to a user, by a user interface, a questionnaire and a set of questionnaire controls, receiving a set of response parameters entered by said user using said questionnaire controls, wherein said plurality of parameters comprises said response parameters.

22. The method according to claim 19, wherein said plurality of parameters comprises at least one parameter extracted from a body liquid test applied to said at least one of said parent and said sibling.

23. The method according to claim 19, wherein said plurality of parameters comprises a parameter pertaining to a body-mass-index of said sibling.

24. The method according to claim 19, wherein said plurality of parameters comprises a parameter pertaining to a body-mass-index of a father of said unborn subject.

25. The method according to claim 19, wherein said plurality of parameters comprises at least 10 of the parameters listed in Table 1.2.

26. A method of predicting likelihood for childhood obesity, comprising:
presenting on a user interface a questionnaire and a set of questionnaire controls, and receiving from said user interface a set of response parameters entered using said questionnaire controls, wherein said set of response parameters characterizes an infant or toddler subject;
accessing a computer readable medium storing a machine learning procedure trained for predicting likelihoods for childhood obesity;
feeding said procedure with said set of parameters; and
receiving from said procedure an output indicative of a likelihood that said infant or toddler subject is expected to develop childhood obesity, wherein said output is related non-linearly to said parameters.
